(19) United States
(12) Patent Application Publication
(10) Pub. No.: US 2012/0184465 A1
(43) Pub. Date: Jul. 19, 2012

(54) COMBINATORIAL METHODS FOR OPTIMIZING ENGINEERED MICROORGANISM FUNCTION

(75) Inventors: Stephen Picataggio, Carlsbad, CA (US); Kirsty Anne Lily Salmon, Carlsbad, CA (US)

(73) Assignee: VERDEZYNE, INC., Carlsbad, CA (US)

(21) Appl. No.: 13/386,010

(22) PCT Filed: Jul. 16, 2010
(86) PCT No.: PCT/US10/42359

§ 371 Date: Mar. 30, 2012

Related U.S. Application Data
(60) Provisional application No. 61/227,058, filed on Jul. 20, 2009.

(51) Int. Cl. C40B 40/08; C12N 5/10; C12N 1/15; C12N 1/19; C12N 15/63; C12N 1/21

(52) U.S. Cl. 506/17; 435/320.1; 435/252.3; 435/254.11; 435/254.2; 435/325; 435/348

(57) ABSTRACT

Described herein are compositions and methods for combinatorial metabolic pathway optimization.

### RELATED PATENT APPLICATION(S)

This patent application is a national stage of international patent application no. PCT/US2010/042359 filed Jul. 16, 2010, entitled COMBINATORIAL METHODS FOR OPTIMIZING ENGINEERED MICROORGANISM FUNCTION, naming Stephen Picataggio and Kirsty Anne Lily Salmon as inventors, and designated by Attorney Docket No. VRD-1003-PC, which claims the benefit of U.S. provisional patent application No. 61/227,058 filed on Jul. 20, 2009, entitled COMBINATORIAL METHODS FOR OPTIMIZING ENGINEERED MICROORGANISM FUNCTION, naming Stephen Picataggio as inventor and designated by Attorney Docket No. VRD-1003-PV. The entire contents of the foregoing patent applications are incorporated herein by reference, including, without limitation, all text, tables and drawings.

### FIELD

The technology relates in part to compositions and methods for improving engineered microorganism function.

### BACKGROUND

Organisms, and in particular, microorganisms, are used to produce biological and chemical products, sometimes with less expense and with less environmental impact than using chemical synthesis or petroleum based chemistries. Some microorganisms offer an advantage of being amenable to genetic manipulation. Microorganisms can be engineered to produce products of interest by harnessing native or modified metabolic pathways, and by introducing novel pathways.

In a given pathway, multiple polypeptides have activities that convert a substrate to a product via a series of intermediates. Many microorganisms have similar if not identical pathways, yet a particular type of activity at a parallel step in a pathway may be carried out with more or less efficiency when comparing two different organisms. For two organisms sharing a common pathway, for example, counterpart polypeptides that gate a parallel activity in the pathway may effect the activity with a different efficiency or different rate. Thus, while related or unrelated organisms may have similar or identical pathways, the efficiency or rate at which each activity is effected may differ among microorganisms.

### SUMMARY

Provided herein are compositions and methods useful for optimizing one or more pathways in an engineered microorganism, and can be utilized to optimize production of a target product by an engineered microorganism. For two or more activities in a pathway, compositions and methods herein provide different combinations of polypeptides that carry out the activities in an organism. Of these, combinations that give rise to efficient production of target product can be identified and selected, thereby producing organisms with optimized production of the target product. Because methods described herein provide multiple combinations of possible pathways, these methods are referred to as “combinatorial methods.”

Thus, featured in some embodiments are methods for generating a combinatorial library of nucleic acids, which comprise: (a) providing a group of polynucleotides comprising two or more polynucleotide subgroups, where, (i) each polynucleotide in each polynucleotide subgroup can encode a polypeptide of a corresponding polypeptide subgroup; (ii) each polypeptide in a particular polypeptide subgroup may share the same type of activity; and (iii) polypeptides of one polypeptide subgroup may have a different type of activity compared to the polypeptides of another polypeptide subgroup; and (b) assembling the polynucleotides into a nucleic acid library.

Also provided in some embodiments are nucleic acid libraries that can comprise a group of polynucleotides that includes two or more polynucleotide subgroups, where (i) each polynucleotide in each polynucleotide subgroup encodes a polypeptide of a corresponding polypeptide subgroup; (ii) each polypeptide in a particular polypeptide subgroup share an activity; and (iii) polypeptides of one polypeptide subgroup have a different activity from the polypeptides of every other polypeptide subgroup. In some embodiments, polypeptides in different subgroups may share a common secondary activity, not in the desired pathway, and in certain embodiments, polypeptides in different subgroups do not share a common activity. In some embodiments, polypeptides in a particular polypeptide subgroup can be related by 60% or greater amino acid sequence identity.

In certain embodiments, each nucleic acid of the nucleic acid libraries described herein can include one polynucleotide species from each of the two or more polynucleotide subgroups. In some embodiments, each nucleic acid of the nucleic acid library can include more than one polynucleotide subgroup from a particular donor organism. That is, in a pathway that has multiple activities an optimized pathway may comprise more than one subgroup from a particular donor organism. In certain embodiments, each polynucleotide of a polynucleotide subgroup can be from a different donor organism type, where a different “type” can refer to a different genus, species, or strain, for example.

In some embodiments, each nucleic acid of the nucleic acid library can comprise polynucleotide species linked in series. In certain embodiments, the polynucleotide species can be separated from one another by linkers. In some embodiments, the polynucleotide species can be in operable linkage with one or more promoters.

In certain embodiments, the polynucleotide species are in operable linkage with one promoter. In related embodiments, polynucleotides in a nucleic acid can be in any suitable order (e.g., subgroups 1, 2, 3 from 5′ to 3′ in one nucleic acid and subgroups 2, 1, 3 from 5′ to 3′ in another nucleic acid.

In some embodiments, each polynucleotide species is in operable linkage with a separate promoter. In related embodiments, a nucleic acid may include a specific promoter operably linked to a specific polynucleotide (e.g., for a nucleic acid containing six polynucleotides, there are six promoters, where each promoter is operably linked to a polynucleotide. In some embodiments, a promoter operably linked to a specific nucleotide may be the same or different for two or more polynucleotides in a nucleic acid. In one non-limiting example, for a nucleic acid containing six polynucleotides, there can be six promoters, each operably linked to a polynucleotide, where (i) all promoters are the same, (ii) all promoters are different, (iii) some promoters are the same and some promoters are different (e.g., 2 promoters are the same and 4 promoters are different).

In certain embodiments, each of the nucleic acid libraries described above includes 60% or more of all possible subgroup species combinations. In some embodiments, there may be 50 or fewer polynucleotide subgroups. That is, there can be 50 or fewer activities that make up one or more related pathways. In certain embodiments, the polynucleotides can be assembled using an oligonucleotide assembly process. In some embodiments, the polynucleotides can comprise complementary DNA (cDNA). In certain embodiments, the polynucleotides can consist essentially of cDNA.

In some embodiments, the methods described above can comprise inserting nucleic acid of the library into an expression construct. In certain embodiments, the method may further comprise inserting the expression construct into an organism. That is, expression constructs bearing nucleic acids from the nucleic acid libraries described herein can be inserted into a host organism, in certain embodiments. In some embodiments, the method can comprise inserting nucleic acid of the library into genomic DNA of an organism. In certain embodiments, the method also can comprise determining the amount of a target product produced by the organism.

In certain embodiments, a method described herein can comprise inserting nucleic acid of the library into a yeast artificial chromosome. In some embodiments, a method described herein may comprise inserting the artificial chromosome in a yeast. In some embodiments, the method also can comprise determining the amount of a target product produced by the yeast.

Provided also in certain embodiments are isolated expression constructs that comprise a nucleic acid from a nucleic acid library produced by methods described herein. Also provided in certain embodiments are organisms that comprise a nucleic acid from a nucleic acid library produced by any of the methods described herein. In some embodiments, an expression construct comprising a nucleic acid from a nucleic acid library produced using methods described herein can be inserted into an organism. In certain embodiments, an organism may comprise an isolated expression construct, constructed as described herein.

In some embodiments, the organism can be a prokaryote. In certain embodiments, the prokaryote can be a bacterium. In some embodiments, the organism can be a eukaryote. In certain embodiments, the eukaryote can be a fungus. In some embodiments, the eukaryote can be yeast. In certain embodiments, the eukaryote can be a mammalian cell. In some embodiments, the eukaryote can be an insect cell.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 depicts a schematic representation of a combinatorial pathway optimization. The theoretical pathway described in FIG. 1 has 3 activities (e.g., labeled Gene 1, Gene 2 and Gene 3) and four possible donor organisms (e.g., shown as the 4 individual horizontal shaded blocks below the words Gene 1, Gene 2, and Gene 3). FIG. 1 also depicts certain steps involved in the method of combinatorial pathway optimization. The “anneal” step adds linkers and/or primers that enable the various naturally occurring or engineered sequences to be cloned into expression constructs or used for amplification, PCR, or primer extension. The primer extension or PCR can further facilitate the combinatorial assembly process, as shown in FIG. 1 where an assembled pathway is depicted and labeled as X^{Y} combinations. The final step shown in FIG. 1 is a measurement of pathway functionality, where substrate is converted to product, and a determination of whether a particular combination of subgroups optimizes pathway functionality in the chosen host organism.

FIG. 2 is a schematic representation of the portion of the lycopene synthesis pathway that was combinatorially optimized and introduced into a host organism. The method and results are presented in Examples 1-3.

FIG. 3 depicts an engineered metabolic pathway that can be used to produce ethanol more efficiently in a host microorganism in which the pathway has been engineered. The solid lines in FIG. 3 represents the metabolic pathway (e.g., Embden-Meyerhoff (EM) pathway) naturally found in a host organism (e.g., Saccharomyces cerevisiae, for example). The dashed lines in FIG. 3 represents a novel activity or pathway engineered (e.g., added, enhanced, optimized, and the like), as described herein, into a microorganism to allow increased ethanol production efficiency. Two activities from the Entner-Doudoroff pathway (ED pathway) have been introduced into a host organism to generate an engineered organism. The introduced activities allow survival with an inactivated EM pathway in addition to increased efficiency of ethanol production. The introduced activities are 2-keto-3-deoxygluconate-6-phosphate aldolase (e.g., EDA) and phosphogluconate dehydratase (e.g., EDD).

FIGS. 4A-4D show DNA and amino acid sequence alignments for the nucleotide sequences of EDA (FIG. 4A, 4B) and EDD (FIG. 4C, 4D) genes from Zymomonas mobilis (native and optimized) and Escherichia coli. The sequences are further described in Example 6. FIGS. 5A and 5B show representative Western blots used to detect levels of various exogenous EDD and EDA gene combinations expressed in a host organism. Experimental conditions and results are described in Example 6. FIG. 6 graphically displays the relative activities of the various EDD/EDA combinations generated as described in Example 7.

FIG. 7 graphically represents the fermentation efficiency of engineered yeast strains carrying exogenous EDD/EDA gene combinations. Vector=p426GPD/p425GPD; EE=EDD-E. coli/EDA-E. coli, EP=EDD-E. coli/EDA-PAO1; PE=EDD-PAO1/EDA-E. coli, PP=EDD-PAO1/EDA-PAO1. Experimental conditions and results are described in Example 8.

FIG. 8 shows a western blot of E. coli crude extract illustrated the presence of the EDD protein at the expected size. Lane 1 is a standard size ladder (Novex Sharp standard), Lane 2 is 1 μg BF1055 cell lysate, Lane 3 is 10 μg BF1055 cell lysate, Lane 4 is 1.5 μg BF1706 cell lysate, Lane 5 is 15 μg BF1706 cell lysate. Experimental methods and results are described in Example 10. FIG. 9 graphically illustrates the results of activity evaluations of EDA genes expressed in yeast. Experimental methods and results are described in Example 10. FIG. 10 graphically illustrated the relative activity of various EDD sources. Experimental methods and results are described in Example 11.

### DETAILED DESCRIPTION

A metabolic pathway can be seen as a series of reaction steps which convert a beginning substrate or element into a final product. Each step is catalyzed by one or more activities. In a pathway where substrate A is converted to end product D, intermediates B and C are produced and converted by specific activities in the pathway. Each specific activity of a pathway can be considered a species of an activity subgroup and a polypeptide that encodes the activity can be considered a species of a counterpart polypeptide subgroup.

As organisms evolve, in different environments and with different selective pressures, the nucleic acid and amino acid sequences of organisms also can evolve and diverge from an ancestral type. Sequence evolution can result in metabolic pathways that may be naturally optimized for a particular organism in a particular environment, which contributes to the genetic diversity of the respective pathways. Changes in nucleotide or amino acid sequences sometimes may cause the efficiency of an activity to be altered (e.g., increase or decrease in the number of number of conversions or energy input/output of the reaction, for example). The changes may have occurred as a result of different selective pressures with which divergently evolving organisms were presented. These selective pressures may have selected for altered activity that allowed the organism containing the altered sequences to function better in a particular environment. These changes increase genetic diversity of similar or identical activities. The evolutionary changes of similar or identical activities can be identified by nucleic acid and/or amino acid sequence comparisons of related activities from organisms with similar or identical pathways. This evolutionary-driven genetic diversity is referred to herein as “natural diversity.”

Commercially useful organisms may have differences in cellular machinery when compared to organisms from which donor activities can be obtained (e.g., transcription and/or translation machinery, for example). An optimized metabolic pathway can be generated for a chosen host organism by combining similar or identical activities from different sources (e.g., natural or engineered genetic diversity), and identifying those combinations that show improvements according to a chosen criteria (e.g., changes in the rate of reaction, changes in yield of reaction, changes in energy requirements for a reaction or efficiency of reaction, and the like or combinations thereof, for example). A host organism can be chosen for its commercial usefulness in fermentation processes or ability to be genetically manipulated, for example. Increasing the efficiency of production of a desired product produced by commercially useful organisms (e.g., microorganisms in a fermentation process, for example) can yield beneficial gains in starting material conversion and profitability.

### Pathway Optimization

Methods described herein can be used to optimize target product formation in an engineered organism. The term “optimization,” and grammatical variants thereof, as used herein, refers to a process whereby a metabolic pathway or portion thereof, is altered using naturally occurring and/or synthesized nucleic acids (e.g., engineered genetic diversity) to increase the rate, yield, and/or production efficiency of a desired end product, when compared to native or reference activities. This type of optimization can be referred to as “combinatorial metabolic pathway engineering” or “combinatorial metabolic engineering”, and is described in further detail herein. Thus, sub-group combinations are generated, the combinations are expressed in organisms, and the organisms then are tested to determine which of the combinations more efficiently or effectively produce a target product, in certain embodiments.

The term “pathway”, “metabolic pathway”, and “catabolic pathway” as described herein, refers to a series of simultaneous or sequential chemical reactions, effected by activities that convert substrates or beginning elements into end compounds or desired products via one or more intermediates. An activity sometimes is conversion of a substrate to an intermediate or product (e.g., catalytic conversion by an enzyme) and sometimes is binding of molecule or ligand, in certain embodiments. The term “identical pathway” as used herein, refers to pathways from related or unrelated organisms that have the same number and type of activities and result in the same end product. The term “similar pathway” as used herein, refers to pathways from related or unrelated organisms that have one or more of: a different number of activities, different types of activities, utilize the same starting or intermediate molecules, and/or result in the same end product. A non-limiting example of similar pathways from different organisms is conversion of xylose to xylulose. The conversion sometimes is performed by a two step process (e.g., a reduction and an oxidation, as found in many yeast, fungus and other eukaryotes, for example), carried out by xylose reductase (XYL1) and xylitol dehydrogenase (XYL2), respectively. In certain organisms, the conversion is performed by a one step process that converts xylose directly to xylulose, as found in many bacteria (e.g., Piromyces, Orpinomyces, Bacteroides thetaiotaomicron, Clostridium phytofermentans, Thermus thermophilus and Ruminococcus flavefaciens are non-limiting examples).

Pathway optimization can be attained, for example, by harnessing naturally occurring genetic diversity and/or engineered genetic diversity. Naturally occurring genetic diversity can be harnessed by testing subgroup polynucleotides from different organisms, in some embodiments. Engineered genetic diversity can be harnessed by testing subgroup polynucleotides that have been codon-optimized or mutated, for example. For codon-optimized diversity, amino acid codon triplets can be substituted for other codons, and/or certain nucleotide sequences can be added, removed or substituted. In certain embodiments, native codons are substituted for more or less preferred codons. In certain embodiments, pathways can be optimized by substituting a related or similar activity for one or more steps from a similar but not identical pathway. A polynucleotide in a subgroup also may have been genetically altered such that, when encoded, effects an activity different than the activity of a native counterpart that was utilized as a starting material for genetic alteration. Nucleic acid and/or amino acid sequences altered by the hand of a person as known in the art can be referred to as “engineered” genetic diversity.

In some embodiments, each polypeptide in a particular polypeptide subgroup has a certain activity. An activity can convert a particular substrate into a particular product. That is, one polypeptide in a subgroup may convert a first substrate to a first product with more efficiency than it converts a second substrate to a second product, yet it has the same activity as another polypeptide in the same subgroup that also converts the second substrate to the second product. For example, (i) one polypeptide in a subgroup may prefer to convert a six-carbon substrate to product, but with less efficiency also will convert a five-carbon substrate to a product, and (ii) another polypeptide in a subgroup may prefer to convert the same five-carbon substrate to same product; these two polypeptides share the same activity of converting the same five-carbon substrate to the same product. An activity may be binding to a particular molecule in certain embodiments. Thus, the term “same activity” as used herein refers to the same type of activity (e.g., convert a certain substrate into a certain product) without regard to the level of activity, or efficiency, so long as the activity is detectable for both polypeptides. In some embodiments, each polypeptide in a particular polypeptide subgroup binds to a particular molecule (e.g., substrate, ligand and the like).

In certain embodiments, polypeptides in a particular polypeptide subgroup (e.g., equivalent to the activity subgroups, for example) can be related by 60% or greater amino acid sequence identity. That is, polypeptides in a particular polypeptide subgroup can be related by 60% or greater, 61% or greater, 62% or greater, 63% or greater, 64% or greater, 65% or greater, 66% or greater, 67% or greater, 68% or greater, 69% or greater, 70% or greater, 71% or greater, 72% or greater, 73% or greater, 74% or greater, 75% or greater, 76% or greater, 77% or greater, 78% or greater, 79% or greater, 80% or greater, 81% or greater, 82% or greater, 83% or greater, 84% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or greater amino acid sequence identity.

In some embodiments, two polypeptides have a different activity when they each convert a different substrate into a product (e.g., a different or same product), or convert the same substrate into a different product. In certain embodiments, two polypeptides can bind to a different molecule (e.g., substrate, ligand) and have a different activity. In some embodiments, two polypeptides having a different activity do not share a common activity. In some embodiments, polypeptides in different subgroups may share a common secondary activity, not in a pathway being optimized, and in certain embodiments.

Each activity is carried out by a polypeptide encoded by polynucleotide. In some embodiments a complementary polynucleotide (e.g., cDNA) encodes message RNA (mRNA) that in turn encodes a polypeptide. Thus, each activity subgroup can be represented by a polynucleotide subgroup that encodes a polypeptide having a particular activity. In some embodiments, the polynucleotides can comprise complementary DNA (cDNA). In certain embodiments, the polynucleotides can consist essentially of cDNA, which refers to a polynucleotide that includes a DNA sequence that encodes mRNA that encodes a polypeptide, and can include one or more non-coding nucleotide sequences that do not have a promoter or other specific function that regulates the amount of mRNA or polypeptide encoded by the DNA (e.g., one or more flanking sequences brought in from a cloning process). In some embodiments, the polynucleotides can consist of cDNA. Complementary DNA can be a native (i.e., wild-type) polynucleotide from an organism in some embodiments, and can be a codon-optimized or mutated polynucleotide.

In certain embodiments, each nucleic acid of a nucleic acid library can include one polynucleotide species from each of the two or more polynucleotide subgroups. In some embodiments, each nucleic acid of the nucleic acid library can include more than one polynucleotide subgroup from a particular donor organism. That is, in a pathway that has multiple activities, an optimized pathway may comprise more than one subgroup from a particular donor organism. In some embodiments, each nucleic acid includes one subgroup species from all polynucleotide subgroups. In certain embodiments, each polynucleotide of a polynucleotide subgroup can be from a different donor organism type, (e.g., different “type” can refer to a different genus, species, or strain, for example). In some embodiments, there may be 50 or fewer polynucleotide subgroups (e.g., about 2, 3, 4, 5, 6, 7, 8, 9, 10, 11, 12, 13, 14, 15, 16, 17, 18, 19, 20, 25, 30, 35, 40, 45 subgroups) in a library. That is, there can be 50 or fewer activities that make up one or more related pathways.

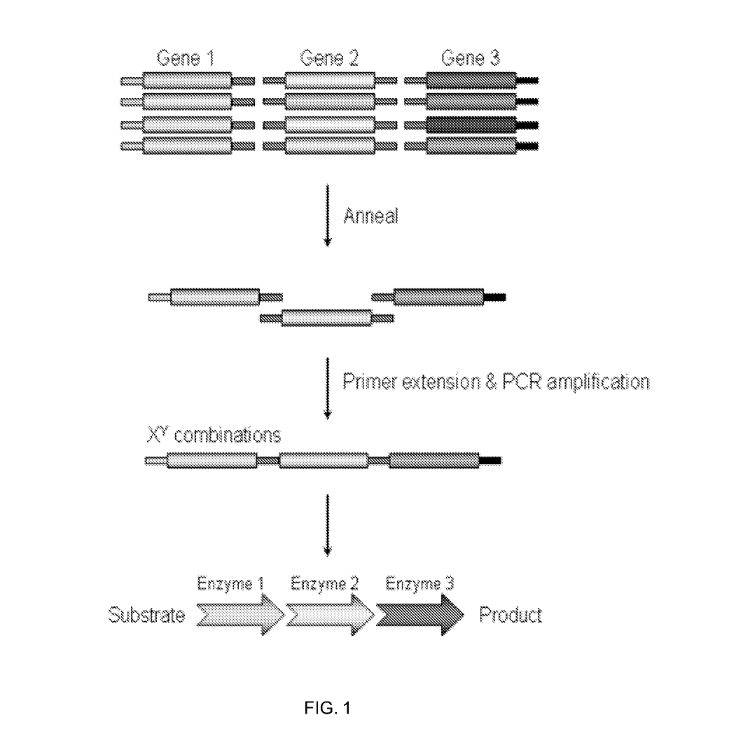

The number of subgroup species combinations is dependent on the number of activities in a given pathway and the number of organisms from which the pathway in question can be isolated. A theoretical example using a three activity subgroup pathway which is found in three organisms is presented here. An example of an engineered pathway (e.g., lycopene biosynthesis) is presented in Examples 1-3. The activities of the theoretical 3 activity pathway are A1, A2, A3, and pathway members can be isolated from three different organisms. The number of combinatorial permutations mathematically is 3 raised to the power 3, or 3 cubed (e.g., 3³), or 27 in this example. A schematic representation of a particular process is shown in FIG. 1. The example depicted in FIG. 1 incorporates a three activity pathway where the activities are isolate from four donor organisms. The number of permutations possible in this example is 3⁴ or 81 possible library combinations.

The number of possible combinations in a library therefore can be represented by the formula (X)^{Y}, in certain embodiments, where X is the number of activity subgroups (or subdomain groups, as described below) and Y is the number of forms (e.g., species) from which the activity can be effected. Species in a subgroup can be selected from the following non-limiting forms, in certain embodiments: codon-optimized form of a polynucleotide from an organism species, mutated form of a polynucleotide from an organism species, and native form of a polynucleotide from a given organism species, for example.

The formula (X)^{Y} is not always indicative of the number of possible combinations in a library. Different subgroups may include different numbers of possible members. For example, one subgroup may include fewer species than another subgroup in some embodiments. One subgroup may include a certain number of native polynucleotides from different organism species and a certain number of engineered polynucleotides (e.g., mutated, codon-optimized versions), and another subgroup may include a fewer or a greater number of each, for example.

Polynucleotide subgroup species can be assembled into a nucleic acid, which may be, or may be inserted into, an expression construct or nucleic acid reagent. An expression construct often contains one or more regulatory elements (e.g., promoter) that can facilitate production of a polypeptide from a polynucleotide. Such nucleic acids, nucleic acid reagents and expression constructs can be part of a nucleic acid library.

In certain embodiments, polynucleotides can be assembled into a nucleic acid using an assembly process. Any suitable assembly process may be utilized. In certain embodiments, full-length coding sequences in polynucleotides are utilized as building blocks for assembly. Non-limiting examples of such a process is an oligonucleotide assembly process (e.g., described in U.S. Pat. No. 7,262,031 (Lathrop) or Gibson et al, “Enzymatic assembly of DNA molecules up to several hundred kilobases”, Nature Methods, 6(5):343-345, May 2009 and supplemental online methods DOI:10.1038/nmeth.1318). The polynucleotides may be linked in series in a nucleic acid, and in such embodiments, there may or may not be intervening sequences between the polynucleotides. Intervening sequences can include, without limitation, one or more of: a promoter (and/or other regulatory sequence), a linker, a sequence for recombination into genomic DNA of an organism, a gene encoding a selectable marker, a sequence that controls replication of the nucleic acid, a stop codon, a termination sequence and the like. All polynucleotides may not be linked in series in certain embodiments, where one polynucleotide, or a subset of polynucleotides (e.g., two or more) are in a nucleic acid. In some embodiments, the polynucleotides may not be linked in series.

A combinatorial nucleic acid library can contain substantially all possible combinations of subgroup polynucleotides, in some embodiments. In certain embodiments, nucleic acid libraries include a subset of all possible combinations, and in certain embodiments, a library includes 60% or more of all possible subgroup species combinations (e.g., about 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more of all possible subgroup species combinations).

Nucleic acid in a library can be in any suitable form, including, without limitation, linear, circular, plasmid, artificial chromosome and the like. A library can include any suitable number of nucleic acid species, and can include, without limitation, in some embodiments about 20 to about 1,000,000 nucleic acid species (e.g., about 50, 100, 200, 300, 400, 500, 600, 700, 800, 900, 1000, 5000, 10000, 50000, 100000, 500000 nucleic acid species). There may be multiple copy numbers of each nucleic acid species in a library. Nucleic acid species in a library may be separated for further analysis. For example, a nucleic acid library may be inserted into a population of organisms, and individual organisms can include one nucleic acid species. In some embodiments, an individual organism includes two or more nucleic acid species. Individual organisms can be isolated and tested for target product production in certain embodiments, and the individual organisms can be proliferated after isolation and before testing in some embodiments. Thus, in certain applications, the number of nucleic acid species that can be analyzed in a library is limited by the methodology utilized to separate nucleic acid species into organisms and isolate the organisms and nucleic acid species.

After a combinatorial library is constructed, optimized species in the library can be selected. Any suitable assay system can be utilized, include a system that assesses the relative, or actual amount, of a target product produced by a library species. Assay systems amenable to higher-throughput screening often is utilized to select library species that most effectively and/or efficiently produce target product. Assays may be conducted over a time course to determine library species that most quickly produce product, and identify library species that produce the most amount of product.

In addition to metabolic pathway optimization, the combinatorial pathway engineering method also can be used to optimize individual subgroup activities. Each subgroup activity, represented by a polypeptide, can be further divided into individual polypeptide domains. The polypeptide domains can represent all or a portion of known activity centers, contact residues and the like.

Oligonucleotides encoding codon optimized versions of the amino acids in each subdomain from each organism also can be synthesized and assembled in various combinations to further optimize individual activity subgroups, in some embodiments. In certain embodiments, conventional recombinant DNA methods (e.g., cloning, PCR, library construction and the like, for example) can be used to generate the polypeptide subdomain libraries for each activity subgroup. By using recombinant DNA techniques available to one of skill in the art, or oligos of a particular target length and configuration to allow self assembly, various regions of each activity may be further optimized by combining the polypeptide subdomains together in various combinations and assessing which combinations of subdomain regions yields the desired result.

### Organisms

An organism selected often is suitable for genetic manipulation and often can be cultured at cell densities useful for industrial production of a target product. In some embodiments, an organism selected sometimes can be a microorganism. A microorganism selected often can be maintained in a fermentation device. The term “organism” refers to a prokaryotic, archaebacterial or eukaryotic organism, or cells there from, visible to the naked eye or using non-microscopic magnification techniques. The term “microorganism” as used herein refers to a prokaryotic, archaebacterial or eukaryotic organisms or cells there from, visible using microscopic magnification techniques. The terms organism and microorganism can be used interchangeably throughout the document.

The term “engineered organism” or “engineered microorganism” as used herein refers to a modified organism or microorganism that includes one or more activities distinct from an activity present in an organism utilized as a starting point (hereafter a “host microorganism”). An engineered microorganism includes a heterologus polynucleotide in some embodiments, and in certain embodiments, an engineered organism has been subjected to selective conditions that alter an activity, or introduce an activity, relative to the host microorganism. Thus, an engineered microorganism has been altered directly or indirectly by a human being. A host microorganism sometimes is a native microorganism, and at times is an organism that has been engineered to a certain point.

In some embodiments an engineered microorganism is a single cell organism, often capable of dividing and proliferating. A microorganism can include one or more of the following features: aerobe, anaerobe, filamentous, non-filamentous, monoploid, dipoid, auxotrophic and/or non-auxotrophic. In certain embodiments, an engineered microorganism is a prokaryotic microorganism (e.g., bacterium), and in certain embodiments, an engineered microorganism is a non-prokaryotic microorganism. In some embodiments, an engineered microorganism is a eukaryotic microorganism (e.g., yeast, fungi, amoeba, and algae).

Any suitable yeast may be selected as a host microorganism, engineered microorganism or source for a heterologus polynucleotide. Yeast include, but are not limited to, Yarrowia yeast (e.g., Y. lipolytica (formerly classified as Candida lipolytica)), Candida yeast (e.g., C. revkaufi, C. pulcherrima, C. tropicalis, C. utilis), Rhodotorula yeast (e.g., R. glutinus, R. graminis), Rhodosporidium yeast (e.g., R. toruloides), Saccharomyces yeast (e.g., S. cerevisiae, S. bayanus, S. pastorianus, S. carlsbergensis), Cryptococcus yeast, Trichosporon yeast (e.g., T. pullans, T. cutaneum), Pichia yeast (e.g., P. pastoris) and Lipomyces yeast (e.g., L. starkeyii, L. lipoferus). In some embodiments, a yeast is a S. cerevisiae strain including, but not limited to, YGR240CBY4742 (ATCC accession number 4015893) and BY4742 (ATCC accession number 201389). In some embodiments, a yeast is a Y. lipolytica strain that includes, but is not limited to, ATCC20362, ATCC8862, ATCC18944, ATCC20228, ATCC76982 and LGAM S(7)1 strains (Papanikolaou S., and Aggelis G., Bioresour. Technol. 82(1):43-9 (2002)). In certain embodiments, a yeast is a C. tropicalis strain that includes, but is not limited to, ATCC20336, ATCC20913, SU-2 (ura3-/ura3-), ATCC20962, H5343 (beta oxidation blocked; U.S. Pat. No. 5,648,247) strains.

Any suitable fungus may be selected as a host microorganism, engineered microorganism or source for a heterologus polynucleotide. Non-limiting examples of fungi include, but are not limited to, Aspergillus fungi (e.g., A. parasiticus, A. nidulans), Thraustochytrium fungi, Schizochytrium fungi and Rhizopus fungi (e.g., R. arrhizus, R. oryzae, R. nigricans). In some embodiments, a fungus is an A. parasiticus strain that includes, but is not limited to, strain ATCC24690, and in certain embodiments, a fungus is an A. nidulans strain that includes, but is not limited to, strain ATCC38163.

Any suitable algae may be selected as a host microorganism, engineered microorganism or source for a heterologous polynucleotide. Non-limiting examples of algae include, but are not limited to, microalgae (e.g., phytoplankton, microphytes, Spirulina, Chlorella, Chondrus, Mastocarpus, Ulva, Alaria, Cyanobacteria (e.g., blue-green algae) and the like) and macroalgae (e.g., seaweeds, Porphyra, Palmaria and the like).

Any suitable prokaryote may be selected as a host microorganism, engineered microorganism or source for a heterologus polynucleotide. A Gram negative or Gram positive bacteria may be selected. Examples of bacteria include, but are not limited to, Bacillus bacteria (e.g., B. subtilis, B. megaterium), Acinetobacter bacteria, Norcardia baceteria, Xanthobacter bacteria, Escherichia bacteria (e.g., E. coli (e.g., strains DH10B, Stb12, DH5-alpha, DB3, DB3.1), DB4, DB5, JDP682 and ccdA-over (e.g., U.S. application Ser. No. 09/518,188))), Streptomyces bacteria, Erwinia bacteria, Klebsiella bacteria, Serratia bacteria (e.g., S. marcessans), Pseudomonas bacteria (e.g., P. aeruginosa), Salmonella bacteria (e.g., S. typhimurium, S. typhi). Bacteria also include, but are not limited to, photosynthetic bacteria (e.g., green non-sulfur bacteria (e.g., Choroflexus bacteria (e.g., C. aurantiacus), Chloronema bacteria (e.g., C. gigateum)), green sulfur bacteria (e.g., Chlorobium bacteria (e.g., C. limicola), Pelodictyon bacteria (e.g., P. luteolum), purple sulfur bacteria (e.g., Chromatium bacteria (e.g., C. okenii)), and purple non-sulfur bacteria (e.g., Rhodospirillum bacteria (e.g., R. rubrum), Rhodobacter bacteria (e.g., R. sphaeroides, R. capsulatus), and Rhodomicrobium bacteria (e.g., R. vanellii)).

Cells from non-microbial organisms can be utilized as a host microorganism, engineered microorganism or source for a heterologus polynucleotide. Examples of such cells, include, but are not limited to, insect cells (e.g., Drosophila (e.g., D. melanogaster), Spodoptera (e.g., S. frugiperda Sf9 or Sf21 cells) and Trichoplusa (e.g., High-Five cells); nematode cells (e.g., C. elegans cells); avian cells; amphibian cells (e.g., Xenopus laevis cells); reptilian cells; and mammalian cells (e.g., NIH3T3, 293, CHO, COS, VERO, C127, BHK, Per-C6, Bowes melanoma and HeLa cells).

Microorganisms or cells used as host organisms or source for a heterologus polynucleotide are commercially available. Microorganisms and cells described herein, and other suitable microorganisms and cells are available, for example, from Invitrogen Corporation, (Carlsbad, Calif.), American Type Culture Collection (Manassas, Va.), and Agricultural Research Culture Collection (NRRL; Peoria, Ill.).

In certain embodiments, an expression construct comprising a nucleic acid from a nucleic acid library produced using methods described herein can be inserted into an organism. In certain embodiments, an organism may comprise an isolated expression construct, constructed as described herein. In some embodiments, the method can comprise inserting nucleic acid of the library into genomic DNA of an organism. In certain embodiments, the methods described above can comprise inserting nucleic acid of the library into a yeast artificial chromosome.

Host microorganisms and engineered microorganisms may be provided in any suitable form. For example, such microorganisms may be provided in liquid culture or solid culture (e.g., agar-based medium), which may be a primary culture or may have been passaged (e.g., diluted and cultured) one or more times. Microorganisms also may be provided in frozen form or dry form (e.g., lyophilized). Microorganisms may be provided at any suitable concentration.

### Activities

Activity subgroups of chosen pathways can be modified to generate microorganisms engineered to allow a method of independently regulating or controlling (e.g., ability to independently turn on or off, or increase or decrease, for example) the activities in a given metabolic pathway. In some embodiments, regulated control of a desired activity can be the result of a genetic modification. In certain embodiments, the genetic modification can be modification of a promoter sequence. In some embodiments the modification can increase of decrease an activity encoded by a gene operably linked to the promoter element. In certain embodiments, the modification to the promoter element can add or remove a regulatory sequence. In some embodiments the regulatory sequence can respond to a change in environmental or culture conditions. Non-limiting examples of culture conditions that could be used to regulate an activity in this manner include, temperature, light, oxygen, salt, metals and the like. Additional methods for altering an activity by modification of a promoter element are given below.

In some embodiments, the genetic modification can be to an ORF. In certain embodiments, the modification of the ORF can increase or decrease expression of the ORF. In some embodiments modification of the ORF can alter the efficiency of translation of the ORF. In certain embodiments, modification of the ORF can alter the activity of the polypeptide or protein encoded by the ORF. Additional methods for altering an activity by modification of an ORF are given below.

In some embodiments, the pathway optimization can be combined with conditional cell division cycle mutants (e.g., cell division cycle or CDC activity, for example), to allow continued production of a desired product without continued resources being directed to increasing biomass and using energy. In certain embodiments the cell division cycle activity can be thymidylate synthase activity. In certain embodiments, regulated control of cell division can be the result of a genetic modification. In some embodiments, the genetic modification can be to a nucleotide sequence that encodes thymidylate synthase. In certain embodiments, the genetic modification can temporarily inactivate thymidylate synthase activity by rendering the activity temperature sensitive (e.g., heat resistant, heat sensitive, cold resistant, cold sensitive and the like).

In some embodiments, the genetic modification can modify a promoter sequence operably linked to a gene encoding an activity involved in the desired pathway, a gene encoding an activity in control of cell division, or both. In some embodiments the modification can increase of decrease an activity encoded by a gene operably linked to the promoter element. In certain embodiments, the modification to the promoter element can add or remove a regulatory sequence. In some embodiments the regulatory sequence can respond to a change in environmental or culture conditions. Non-limiting examples of culture conditions that could be used to regulate an activity in this manner include, temperature, light, oxygen, salt, metals and the like.

An example of a pathway whose activities have been optimized is presented in Examples 1-3. The pathway used in Examples 1-3 is the lycopene biosynthesis pathway which comprises 3 activities encoded by 3 genes; CrtE, CrtI and CrtB. The genes encoding the three activities of the pathway were isolated from Pantoea ananatis, Pantoea agglomerans and Chronobacter sakazakii. In some embodiments, an engineered microorganism comprising one or more activities described above or below, and optionally further comprising modifications to promoter elements, 5′ UTR or 3′ UTR sequences can be used in to produce lycopene as described in Example 1-3. Additionally, by inhibiting cell growth and cell division by use of a temperature sensitive cell division control activity while allowing cellular fermentation to proceed, significant increases in lycopene yield may be realized when compared to unmodified lycopene pathway activities in the chosen host organism.

### Polynucleotides and Polypeptides

A nucleic acid (e.g., also referred to herein as nucleic acid reagent, target nucleic acid, target nucleotide sequence, nucleotide sequence of interest or nucleic acid region of interest) can be from any source or composition, such as DNA, cDNA, gDNA (genomic DNA), RNA, siRNA (short inhibitory RNA), RNAi, tRNA or mRNA, for example, and can be in any form (e.g., linear, circular, supercoiled, single-stranded, double-stranded, and the like). A nucleic acid can also comprise DNA or RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Deoxyribonucleotides include deoxyadenosine, deoxycytidine, deoxyguanosine and deoxythymidine. For RNA, the uracil base is uridine.

In some embodiments, nucleic acids can be used to make nucleic acid libraries and/or combinatorial nucleic acid libraries. In some embodiments, the methods described herein can comprise inserting nucleic acid of the library into an expression construct or nucleic acid reagent. In certain embodiments, the nucleic acid libraries described herein can be combined with or made part of a nucleic acid reagent using standard recombinant DNA methods available to one of skill in the art, or as described herein. In some embodiments, each nucleic acid of the nucleic acid library can comprise polynucleotide species linked in series. In certain embodiments, the polynucleotide species can be separated from one another by linkers.

A nucleic acid sometimes is a plasmid, phage, autonomously replicating sequence (ARS), centromere, artificial chromosome, yeast artificial chromosome (e.g., YAC) or other nucleic acid able to replicate or be replicated in a host cell. In certain embodiments a nucleic acid can be from a library or can be obtained from enzymatically digested, sheared or sonicated genomic DNA (e.g., fragmented) from an organism of interest. In some embodiments, nucleic acid subjected to fragmentation or cleavage may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000 or 10000 base pairs. Fragments can be generated by any suitable method in the art, and the average, mean or nominal length of nucleic acid fragments can be controlled by selecting an appropriate fragment-generating procedure by the person of ordinary skill. In some embodiments, the fragmented DNA can be size selected to obtain nucleic acid fragments of a particular size range. In some embodiments, a nucleic acid library as described herein can be inserted into an expression construct. In certain embodiments, a nucleic acid library as described herein can be inserted to yeast artificial chromosomes.

Nucleic acid can be fragmented by various methods known to the person of ordinary skill, which include without limitation, physical, chemical and enzymic processes. Examples of such processes are described in U.S. Patent Application Publication No. 20050112590 (published on May 26, 2005, entitled “Fragmentation-based methods and systems for sequence variation detection and discovery,” naming Van Den Boom et al.). Certain processes can be selected by the person of ordinary skill to generate non-specifically cleaved fragments or specifically cleaved fragments. Examples of processes that can generate non-specifically cleaved fragment sample nucleic acid include, without limitation, contacting sample nucleic acid with apparatus that expose nucleic acid to shearing force (e.g., passing nucleic acid through a syringe needle; use of a French press); exposing sample nucleic acid to irradiation (e.g., gamma, x-ray, UV irradiation; fragment sizes can be controlled by irradiation intensity); boiling nucleic acid in water (e.g., yields about 500 base pair fragments) and exposing nucleic acid to an acid and base hydrolysis process.

Nucleic acid may be specifically cleaved by contacting the nucleic acid with one or more specific cleavage agents. The term “specific cleavage agent” as used herein refers to an agent, sometimes a chemical or an enzyme that can cleave a nucleic acid at one or more specific sites. Specific cleavage agents often will cleave specifically according to a particular nucleotide sequence at a particular site. Examples of enzymic specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I. Bgl II, Bln I, Bsm I, BssH II, BstE II, Cfo I, Cla I, Dde I, Dpn I, Dra I, EcIX I, EcoR I, EcoR I, EcoR II, EcoR V, Hae II, Hae II, Hind III, Hind III, Hpa I, Hpa II, Kpn I, Ksp I, Mlu I, MluN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I); glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease III); ribozymes, and DNAzymes. Sample nucleic acid may be treated with a chemical agent, or synthesized using modified nucleotides, and the modified nucleic acid may be cleaved. In non-limiting examples, sample nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

As used herein, the term “complementary cleavage reactions” refers to cleavage reactions that are carried out on the same nucleic acid using different cleavage reagents or by altering the cleavage specificity of the same cleavage reagent such that alternate cleavage patterns of the same target or reference nucleic acid or protein are generated. In certain embodiments, nucleic acids of interest may be treated with one or more specific cleavage agents (e.g., 1, 2, 3, 4, 5, 6, 7, 8, 9, 10 or more specific cleavage agents) in one or more reaction vessels (e.g., nucleic acid of interest is treated with each specific cleavage agent in a separate vessel).

A nucleic acid suitable for use in the embodiments described herein sometimes is amplified by any amplification process known in the art (e.g., PCR, RT-PCR and the like). Nucleic acid amplification may be particularly beneficial when using organisms that are typically difficult to culture (e.g., slow growing, require specialize culture conditions and the like). The terms “amplify”, “amplification”, “amplification reaction”, or “amplifying” as used herein refer to any in vitro processes for multiplying the copies of a target sequence of nucleic acid. Amplification sometimes refers to an “exponential” increase in target nucleic acid. However, “amplifying” as used herein can also refer to linear increases in the numbers of a select target sequence of nucleic acid, but is different than a one-time, single primer extension step. In some embodiments, a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may also limit inaccuracies associated with depleted reactants in standard PCR reactions. In some embodiments, amplification and/or PCR can be used to add linkers or “sticky-ends” to nucleotide sequences in a combinatorial library to facilitate assembly of combinatorial pathways and/or facilitate inserting assembled pathways into expression constructions of nucleic acid reagents.

In some embodiments, a nucleic acid reagent sometimes is stably integrated into the chromosome of the host organism, or a nucleic acid reagent can be a deletion of a portion of the host chromosome, in certain embodiments (e.g., genetically modified organisms, where alteration of the host genome confers the ability to selectively or preferentially maintain the desired organism carrying the genetic modification). Such nucleic acid reagents (e.g., nucleic acids or genetically modified organisms whose altered genome confers a selectable trait to the organism) can be selected for their ability to guide production of a desired protein or nucleic acid molecule. When desired, the nucleic acid reagent can be altered such that codons encode for (i) the same amino acid, using a different tRNA than that specified in the native sequence, or (ii) a different amino acid than is normal, including unconventional or unnatural amino acids (including detectably labeled amino acids). As described herein, the term “native sequence” refers to an unmodified nucleotide sequence as found in its natural setting (e.g., a nucleotide sequence as found in an organism).

A nucleic acid or nucleic acid reagent can comprise certain elements often selected according to the intended use of the nucleic acid. Any of the following elements can be included in or excluded from a nucleic acid reagent. A nucleic acid reagent, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and one or more selection elements. A nucleic acid reagent can be provided with one or more of such elements and other elements may be inserted into the nucleic acid before the nucleic acid is introduced into the desired organism. In some embodiments, a provided nucleic acid reagent comprises a promoter, 5′UTR, optional 3′UTR and insertion element(s) by which a target nucleotide sequence is inserted (i.e., cloned) into the nucleotide acid reagent. In certain embodiments, a provided nucleic acid reagent comprises a promoter, insertion element(s) and optional 3′UTR, and a 5′ UTR/target nucleotide sequence is inserted with an optional 3′UTR. The elements can be arranged in any order suitable for expression in the chosen expression system (e.g., expression in a chosen organism, or expression in a cell free system, for example), and in some embodiments a nucleic acid reagent comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR.

A promoter element typically is required for DNA synthesis and/or RNA synthesis. A promoter element often comprises a region of DNA that can facilitate the transcription of a particular gene, by providing a start site for the synthesis of RNA corresponding to a gene. Promoters generally are located near the genes they regulate, are located upstream of the gene (e.g., 5′ of the gene), and are on the same strand of DNA as the sense strand of the gene, in some embodiments.

A promoter often interacts with a RNA polymerase. A polymerase is an enzyme that catalyses synthesis of nucleic acids using a preexisting nucleic acid reagent. When the template is a DNA template, an RNA molecule is transcribed before protein is synthesized. Enzymes having polymerase activity suitable for use in the present methods include any polymerase that is active in the chosen system with the chosen template to synthesize protein. In some embodiments, a promoter (e.g., a heterologus promoter) also referred to herein as a promoter element, can be operably linked to a nucleotide sequence or an open reading frame (ORF). Transcription from the promoter element can catalyze the synthesis of an RNA corresponding to the nucleotide sequence or ORF sequence operably linked to the promoter, which in turn leads to synthesis of a desired peptide, polypeptide or protein. The term “operably linked” as used herein with respect to promoters refers to a nucleotide sequence (e.g., a coding sequence) present on the same nucleic acid molecule as a promoter element and whose expression is under the control of said promoter element.

Promoter elements sometimes exhibit responsiveness to regulatory control. Promoter elements also sometimes can be regulated by a selective agent. That is, transcription from promoter elements sometimes can be turned on, turned off, up-regulated or down-regulated, in response to a change in environmental, nutritional or internal conditions or signals (e.g., heat inducible promoters, light regulated promoters, feedback regulated promoters, hormone influenced promoters, tissue specific promoters, oxygen and pH influenced promoters, promoters that are responsive to selective agents (e.g., kanamycin) and the like, for example). Promoters influenced by environmental, nutritional or internal signals frequently are influenced by a signal (direct or indirect) that binds at or near the promoter and increases or decreases expression of the target sequence under certain conditions.

Non-limiting examples of selective or regulatory agents that can influence transcription from a promoter element used in embodiments described herein include, without limitation, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like). In some embodiments, the regulatory or selective agent can be added to change the existing growth conditions to which the organism is subjected (e.g., growth in liquid culture, growth in a fermentor, growth on solid nutrient plates and the like for example).

In some embodiments, regulation of a promoter element can be used to alter (e.g., increase, add, decrease or substantially eliminate) the activity of a peptide, polypeptide or protein (e.g., enzyme activity for example). For example, a microorganism can be engineered by genetic modification to express a nucleic acid reagent that can add a novel activity (e.g., an activity not normally found in the host organism) or increase the expression of an existing activity by increasing transcription from a homologous or heterologus promoter operably linked to a nucleotide sequence of interest (e.g., homologous or heterologus nucleotide sequence of interest), in certain embodiments. In some embodiments, a microorganism can be engineered by genetic modification to express a nucleic acid reagent that can decrease expression of an activity by decreasing or substantially eliminating transcription from a homologous or heterologus promoter operably linked to a nucleotide sequence of interest, in certain embodiments.

In some embodiments, a polynucleotide species, as described herein, can be in operable linkage with one or more promoters. In certain embodiments, the polynucleotide species are in operable linkage with one promoter. In some embodiments, each polynucleotide species is in operable linkage with a separate promoter. In certain embodiments, each polynucleotide is operably linked to its own dedicated promoter. Non-limiting examples of constitutive promoters suitable for expression of an optimized metabolic pathway (e.g., the lycopene pathway, for example) include; XT7-390 (e.g., the first 390 nt of the HXT7 promoter), GPD1 (e.g., NAD-dependent glycerol-3-phosphate dehydrogenase, also known as DAR1, HOR1, OSG1 and OSR5), TEF1 (e.g., transcription elongation factor-1), PGK1 (e.g., Phosphoglycerate kinase-1), ADH1 (e.g., Alcohol dehydrogenase-1), PMA1 (e.g., Plasma membrane H+-ATPase also known as KTI10) and the like.

The term “constitutive promoters suitable for expression” as used herein refers to the strength of the promoter and the ability of the promoter to initiate sufficient rounds of transcription that an activity or nucleic acid encoding the activity can be detected (e.g., mRNA from the gene and/or the activity associated with the gene, for example). Thus, the promoter or promoters chosen are chosen due to their ability to initiate sufficient rounds of transcription that the desired pathway activities are present in sufficient quantity to produce acceptable levels of the desired end product. In some embodiments, promoters responsive to changes in the growth medium or environment (e.g., regulatable promoters or conditionally regulated promoters for example) can be used to express nucleic acids from nucleic acid libraries constructed according to methods described herein.

Tables herein provide non-limiting lists of yeast promoters that are up-regulated by oxygen, yeast promoters that are down-regulated by oxygen, yeast transcriptional repressors and their associated genes, DNA binding motifs as determined using the MEME sequence analysis software. Potential regulator binding motifs can be identified using the program MEME to search intergenic regions bound by regulators for overrepresented sequences. For each regulator, the sequences of intergenic regions bound with p-values less than 0.001 were extracted to use as input for motif discovery. The MEME software was run using the following settings: a motif width ranging from 6 to 18 bases, the “zoops” distribution model, a 6th order Markov background model and a discovery limit of 20 motifs. The discovered sequence motifs were scored for significance by two criteria: an E-value calculated by MEME and a specificity score. The motif with the best score using each metric is shown for each regulator. All motifs presented are derived from datasets generated in rich growth conditions with the exception of a previously published dataset for epitope-tagged Gal4 grown in galactose.

In some embodiments, the altered activity can be found by screening the organism under conditions that select for the desired change in activity. For example, certain microorganisms can be adapted to increase or decrease an activity by selecting or screening the organism in question on a media containing substances that are poorly metabolized or even toxic. An increase in the ability of an organism to grow a substance that is normally poorly metabolized would result in an increase in the growth rate on that substance, for example. A decrease in the sensitivity to a toxic substance might be manifested by growth on higher concentrations of the toxic substance, for example. Genetic modifications that are identified in this manner sometimes are referred to as naturally occurring mutations or the organisms that carry them can sometimes be referred to as naturally occurring mutants. Modifications obtained in this manner are not limited to alterations in promoter sequences. That is, screening microorganisms by selective pressure, as described above, can yield genetic alterations that can occur in non-promoter sequences, and sometimes also can occur in sequences that are not in the nucleotide sequence of interest, but in a related nucleotide sequences (e.g., a gene involved in a different step of the same pathway, a transport gene, and the like). Naturally occurring mutants sometimes can be found by isolating naturally occurring variants from unique environments, in some embodiments.

In addition to the regulated promoter sequences, regulatory sequences, and coding polynucleotides provided herein or useable with the methods described herein, a nucleic acid reagent may include a polynucleotide sequence 80% or more identical to the foregoing (or to the complementary sequences). That is, a nucleotide sequence that is at least 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to a nucleotide sequence described herein can be utilized. The term “identical” as used herein refers to two or more nucleotide sequences having substantially the same nucleotide sequence when compared to each other. One test for determining whether two nucleotide sequences or amino acids sequences are substantially identical is to determine the percent of identical nucleotide sequences or amino acid sequences shared.

Calculations of sequence identity can be performed as follows. Sequences are aligned for optimal comparison purposes (e.g., gaps can be introduced in one or both of a first and a second amino acid or nucleotide sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Sequence identity can also be determined by hybridization assays conducted under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

As noted above, nucleic acid reagents may also comprise one or more 5′ UTR's, and one or more 3′UTR's. A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the chosen expression system (e.g., expression in a chosen organism, or expression in a cell free system, for example). A 5′ UTR sometimes comprises one or more of the following elements known to the artisan: enhancer sequences (e.g., transcriptional or translational), transcription initiation site, transcription factor binding site, translation regulation site, translation initiation site, translation factor binding site, accessory protein binding site, feedback regulation agent binding sites, Pribnow box, TATA box, −35 element, E-box (helix-loop-helix binding element), ribosome binding site, replicon, internal ribosome entry site (IRES), silencer element and the like. In some embodiments, a promoter element may be isolated such that all 5′ UTR elements necessary for proper conditional regulation are contained in the promoter element fragment, or within a functional subsequence of a promoter element fragment.

A 5′ UTR in the nucleic acid reagent can comprise a translational enhancer nucleotide sequence. A translational enhancer nucleotide sequence often is located between the promoter and the target nucleotide sequence in a nucleic acid reagent. A translational enhancer sequence often binds to a ribosome, sometimes is an 18S rRNA-binding ribonucleotide sequence (i.e., a 40S ribosome binding sequence) and sometimes is an internal ribosome entry sequence (IRES). An IRES generally forms an RNA scaffold with precisely placed RNA tertiary structures that contact a 40S ribosomal subunit via a number of specific intermolecular interactions. Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); Gallie, Nucleic Acids Research 30: 3401-3411 (2002); Shaloiko et al., http address www.interscience.wiley.com, DOI: 10.1002/bit.20267; and Gallie et al., Nucleic Acids Research 15: 3257-3273 (1987)).

A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′ UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the nucleic acid reagent as a translational enhancer sequence (e.g., devoid of guanosine nucleotides and includes a 25 nucleotide long poly (CAA) central region).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′UTR based upon the chosen expression system (e.g., expression in a chosen organism, for example). A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

In some embodiments, modification of a 5′ UTR and/or a 3′ UTR can be used to alter (e.g., increase, add, decrease or substantially eliminate) the activity of a promoter. Alteration of the promoter activity can in turn alter the activity of a peptide, polypeptide or protein (e.g., enzyme activity for example), by a change in transcription of the nucleotide sequence(s) of interest from an operably linked promoter element comprising the modified 5′ or 3′ UTR. For example, a microorganism can be engineered by genetic modification to express a nucleic acid reagent comprising a modified 5′ or 3′ UTR that can add a novel activity (e.g., an activity not normally found in the host organism) or increase the expression of an existing activity by increasing transcription from a homologous or heterologus promoter operably linked to a nucleotide sequence of interest (e.g., homologous or heterologus nucleotide sequence of interest), in certain embodiments. In some embodiments, a microorganism can be engineered by genetic modification to express a nucleic acid reagent comprising a modified 5′ or 3′ UTR that can decrease the expression of an activity by decreasing or substantially eliminating transcription from a homologous or heterologus promoter operably linked to a nucleotide sequence of interest, in certain embodiments.

A nucleotide reagent sometimes can comprise a target nucleotide sequence. A “target nucleotide sequence” as used herein encodes a nucleic acid, peptide, polypeptide or protein of interest, and may be a ribonucleotide sequence or a deoxyribonucleotide sequence. A target nucleic acid sometimes is an untranslated ribonucleic acid and sometimes is a translated ribonucleic acid. An untranslated ribonucleic acid may include, but is not limited to, a small interfering ribonucleic acid (siRNA), a short hairpin ribonucleic acid (shRNA), other ribonucleic acid capable of RNA interference (RNAi), an antisense ribonucleic acid, or a ribozyme. A translatable target nucleotide sequence (e.g., a target ribonucleotide sequence) sometimes encodes a peptide, polypeptide or protein, which are sometimes referred to herein as “target peptides,” “target polypeptides” or “target proteins.”

Any peptides, polypeptides or proteins, or an activity catalyzed by one or more peptides, polypeptides or proteins may be encoded by a target nucleotide sequence and may be selected by a person of ordinary skill in the art. Representative proteins include enzymes (e.g., part or all of a metabolic pathway, lycopene biosyntheses, Entner-Doudoroff pathway and the like, for example), antibodies, serum proteins (e.g., albumin), membrane bound proteins, hormones (e.g., growth hormone, erythropoietin, insulin, etc.), cytokines, etc., and include both naturally occurring and exogenously expressed polypeptides. Representative activities (e.g., enzymes or combinations of enzymes which are functionally associated to provide an activity or group of activities as in a metabolic pathway) include any activities associated with a desired metabolic pathway (e.g., GGPP synthase activity, phytoene synthase activity, phytoene desaturase activity for lycopene synthesis and the like, for example). The term “enzyme” as used herein refers to a protein which can act as a catalyst to induce a chemical change in other compounds, thereby producing one or more products from one or more substrates.

Non-limiting examples of specific metabolic pathways (e.g., groups of enzymes or activities) suitable for optimizing, using embodiments described herein, are listed above. It will be understood that the methods and compositions described in embodiments presented herein can be used to; (i) optimize any metabolic pathway that produces a desirable end product, and/or (ii) optimize subdomains within an activity subgroup of a metabolic pathway. The term “protein” as used herein refers to a molecule having a sequence of amino acids linked by peptide bonds. This term includes fusion proteins, oligopeptides, peptides, cyclic peptides, polypeptides and polypeptide derivatives, whether native or recombinant, and also includes fragments, derivatives, homologs, and variants thereof. A protein or polypeptide sometimes is of intracellular origin (e.g., located in the nucleus, cytosol, or interstitial space of host cells in vivo) and sometimes is a cell membrane protein in vivo. In some embodiments (described above, and in further detail below in Engineering and Alteration Methods), a genetic modification can result in a modification (e.g., increase, substantially increase, decrease or substantially decrease) of a target activity.

A translatable nucleotide sequence generally is located between a start codon (AUG in ribonucleic acids and ATG in deoxyribonucleic acids) and a stop codon (e.g., UAA (ochre), UAG (amber) or UGA (opal) in ribonucleic acids and TAA, TAG or TGA in deoxyribonucleic acids), and sometimes is referred to herein as an “open reading frame” (ORF). A nucleic acid reagent sometimes comprises one or more ORFs. An ORF may be from any suitable source, sometimes from genomic DNA, mRNA, reverse transcribed RNA or complementary DNA (cDNA) or a nucleic acid library comprising one or more of the foregoing, and is from any organism species that contains a nucleotide sequence of interest, protein of interest, or activity of interest. Non-limiting examples of organisms from which an ORF can be obtained include algae, bacteria, yeast, fungi, human, insect, nematode, bovine, equine, canine, feline, rat or mouse, for example.

A nucleic acid reagent sometimes comprises a nucleotide sequence adjacent to an ORF that is translated in conjunction with the ORF and encodes an amino acid tag. The tag-encoding nucleotide sequence is located 3′ and/or 5′ of an ORF in the nucleic acid reagent, thereby encoding a tag at the C-terminus or N-terminus of the protein or peptide encoded by the ORF. Any tag that does not abrogate in vitro transcription and/or translation may be utilized and may be appropriately selected by the artisan. Tags may facilitate isolation and/or purification of the desired ORF product from culture or fermentation media.

A tag sometimes specifically binds a molecule or moiety of a solid phase or a detectable label, for example, thereby having utility for isolating, purifying and/or detecting a protein or peptide encoded by the ORF. In some embodiments, a tag comprises one or more of the following elements: FLAG (e.g., DYKDDDDKG), V5 (e.g., GKPIPNPLLGLDST), c-MYC (e.g., EQKLISEEDL), HSV (e.g., QPELAPEDPED), influenza hemaglutinin, HA (e.g., YPYDVPDYA), VSV-G (e.g., YTDIEMNRLGK), bacterial glutathione-S-transferase, maltose binding protein, a streptavidin- or avidin-binding tag (e.g., pcDNA™6 BioEase™ Gateway® Biotinylation System (Invitrogen)), thioredoxin, β-galactosidase, VSV-glycoprotein, a fluorescent protein (e.g., green fluorescent protein or one of its many color variants (e.g., yellow, red, blue)), a polylysine or polyarginine sequence, a polyhistidine sequence (e.g., His6) or other sequence that chelates a metal (e.g., cobalt, zinc, copper), and/or a cysteine-rich sequence that binds to an arsenic-containing molecule. In certain embodiments, a cysteine-rich tag comprises the amino acid sequence CC-Xn-CC, wherein X is any amino acid and n is 1 to 3, and the cysteine-rich sequence sometimes is CCPGCC. In certain embodiments, the tag comprises a cysteine-rich element and a polyhistidine element (e.g., CCPGCC and His6).

A tag often conveniently binds to a binding partner. For example, some tags bind to an antibody (e.g., FLAG) and sometimes specifically bind to a small molecule. For example, a polyhistidine tag specifically chelates a bivalent metal, such as copper, zinc and cobalt; a polylysine or polyarginine tag specifically binds to a zinc finger; a glutathione S-transferase tag binds to glutathione; and a cysteine-rich tag specifically binds to an arsenic-containing molecule. Arsenic-containing molecules include LUMIO™ agents (Invitrogen, California), such as FlAsH™ (EDT2[4′,5′-bis(1,3,2-dithioarsolan-2-yl)fluorescein-(1,2-ethanedithiol)2]) and ReAsH reagents (e.g., U.S. Pat. No. 5,932,474 to Tsien et al., entitled “Target Sequences for Synthetic Molecules;” U.S. Pat. No. 6,054,271 to Tsien et al., entitled “Methods of Using Synthetic Molecules and Target Sequences;” U.S. Pat. Nos. 6,451,569 and 6,008,378; published U.S. Patent Application 2003/0083373, and published PCT Patent Application WO 99/21013, all to Tsien et al. and all entitled “Synthetic Molecules that Specifically React with Target Sequences”). Such antibodies and small molecules sometimes are linked to a solid phase for convenient isolation of the target protein or target peptide.

A tag sometimes comprises a sequence that localizes a translated protein or peptide to a component in a system, which is referred to as a “signal sequence” or “localization signal sequence” herein. A signal sequence often is incorporated at the N-terminus of a target protein or target peptide, and sometimes is incorporated at the C-terminus. Examples of signal sequences are known to the artisan, are readily incorporated into a nucleic acid reagent, and often are selected according to the organism in which expression of the nucleic acid reagent is performed. A signal sequence in some embodiments localizes a translated protein or peptide to a cell membrane. Examples of signal sequences include, but are not limited to, a nucleus targeting signal (e.g., steroid receptor sequence and N-terminal sequence of SV40 virus large T antigen); mitochondrial targeting signal (e.g., amino acid sequence that forms an amphipathic helix); peroxisome targeting signal (e.g., C-terminal sequence in YFG from S. cerevisiae); and a secretion signal (e.g., N-terminal sequences from invertase, mating factor alpha, PHO5 and SUC2 in S. cerevisiae; multiple N-terminal sequences of B. subtilis proteins (e.g., Tjalsma et al., Microbiol. Molec. Biol. Rev. 64: 515-547 (2000)); alpha amylase signal sequence (e.g., U.S. Pat. No. 6,288,302); pectate lyase signal sequence (e.g., U.S. Pat. No. 5,846,818); precollagen signal sequence (e.g., U.S. Pat. No. 5,712,114); OmpA signal sequence (e.g., U.S. Pat. No. 5,470,719); lam beta signal sequence (e.g., U.S. Pat. No. 5,389,529); B. brevis signal sequence (e.g., U.S. Pat. No. 5,232,841); and P. pastoris signal sequence (e.g., U.S. Pat. No. 5,268,273)).

A tag sometimes is directly adjacent to the amino acid sequence encoded by an ORF (i.e., there is no intervening sequence) and sometimes a tag is substantially adjacent to an ORF encoded amino acid sequence (e.g., an intervening sequence is present). An intervening sequence sometimes includes a recognition site for a protease, which is useful for cleaving a tag from a target protein or peptide. In some embodiments, the intervening sequence is cleaved by Factor Xa (e.g., recognition site I (E/D)GR), thrombin (e.g., recognition site LVPRGS), enterokinase (e.g., recognition site DDDDK), TEV protease (e.g., recognition site ENLYFQG) or PreScission™ protease (e.g., recognition site LEVLFQGP), for example.

An intervening sequence sometimes is referred to herein as a “linker sequence,” and may be of any suitable length selected by the artisan. A linker sequence sometimes is about 1 to about 20 amino acids in length, and sometimes about 5 to about 10 amino acids in length. The artisan may select the linker length to substantially preserve target protein or peptide function (e.g., a tag may reduce target protein or peptide function unless separated by a linker), to enhance disassociation of a tag from a target protein or peptide when a protease cleavage site is present (e.g., cleavage may be enhanced when a linker is present), and to enhance interaction of a tag/target protein product with a solid phase. A linker can be of any suitable amino acid content, and often comprises a higher proportion of amino acids having relatively short side chains (e.g., glycine, alanine, serine and threonine).

A “linker” also may be a polynucleotide that separates polynucleotides that encode polypeptides in a nucleic acid. A linker can be of any suitable length, and can be, without limitation, about 200 base pairs or less, about 150 base pairs or less, about 100 base pairs or less or about 50 base pairs or less (e.g., about 10, 20, 30, 40, 50, 60, 70, 80, 90, 100, 110, 120, 130, 140, 150, 160, 170, 180 or 190). A linker often does not include a promoter polynucleotide. A nucleic acid in some embodiments can include a single promoter, a single operon and a single terminator, where the operon includes no linker, or includes one or more linkers between polynucleotides that encode polypeptides. A nucleic acid in certain embodiments may include multiple (e.g., two or more) promoter-polynucleotide units (where the polynucleotide encodes a polypeptide) each separated by a linker.

A nucleic acid reagent sometimes includes a stop codon between a tag element and an insertion element or ORF, which can be useful for translating an ORF with or without the tag. Mutant tRNA molecules that recognize stop codons (described above) suppress translation termination and thereby are designated “suppressor tRNAs.” Suppressor tRNAs can result in the insertion of amino acids and continuation of translation past stop codons (e.g., U.S. Patent Application No. 60/587,583, filed Jul. 14, 2004, entitled “Production of Fusion Proteins by Cell-Free Protein Synthesis,”; Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, D.C.). A number of suppressor tRNAs are known, including but not limited to, supE, supP, supD, supF and supZ suppressors, which suppress the termination of translation of the amber stop codon; supB, gIT, supL, supN, supC and supM suppressors, which suppress the function of the ochre stop codon and glyT, trpT and Su-9 suppressors, which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. Mutations that enhance the efficiency of termination suppressors (i.e., increase stop codon read-through) have been identified. These include, but are not limited to, mutations in the uar gene (also known as the prfA gene), mutations in the ups gene, mutations in the sueA, sueB and sueC genes, mutations in the rpsD (ramA) and rpsE (spcA) genes and mutations in the rplL gene.

Thus, a nucleic acid reagent comprising a stop codon located between an ORF and a tag can yield a translated ORF alone when no suppressor tRNA is present in the translation system, and can yield a translated ORF-tag fusion when a suppressor tRNA is present in the system. Suppressor tRNA can be generated in cells transfected with a nucleic acid encoding the tRNA (e.g., a replication incompetent adenovirus containing the human tRNA-Ser suppressor gene can be transfected into cells, or a YAC containing a yeast or bacterial tRNA suppressor gene can be transfected into yeast cells, for example). Vectors for synthesizing suppressor tRNA and for translating ORFs with or without a tag are available to the artisan (e.g., Tag-On-Demand™ kit (Invitrogen Corporation, California); Tag-On-Demand™ Suppressor Supernatant Instruction Manual, Version B, 6 Jun. 2003, at http address www.invitrogen.com/content/sfs/manuals/tagondemand_supernatant_man.pdf; Tag-On-Demand™ Gateway® Vector Instruction Manual, Version B, 20 June, 2003 at http address www.invitrogen.com/content/sfs/manuals/tagondemand_vectors_man.pdf; and Capone et al., Amber, ochre and opal suppressor tRNA genes derived from a human serine tRNA gene. EMBO J. 4:213, 1985).

Any convenient cloning strategy known in the art may be utilized to incorporate an element, such as an ORF, into a nucleic acid reagent. Known methods can be utilized to insert an element into the template independent of an insertion element, such as (1) cleaving the template at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the nucleic acid reagent, such as an oligonucleotide primer hybridization site for PCR, for example, and others described hereafter. In some embodiments, a cloning strategy can be combined with genetic manipulation such as recombination (e.g., recombination of a nucleic acid reagent with a nucleotide sequence of interest into the genome of the organism to be modified, as described further below). In some embodiments, the cloned ORF(s) can produce (directly or indirectly) lycopene, by engineering a microorganism with one or more ORFs of interest, which microorganism comprises one or more altered activities selected from the group consisting of:

In some embodiments, the nucleic acid reagent includes one or more recombinase insertion sites. A recombinase insertion site is a recognition sequence on a nucleic acid molecule that participates in an integration/recombination reaction by recombination proteins. For example, the recombination site for Cre recombinase is loxP, which is a 34 base pair sequence comprised of two 13 base pair inverted repeats (serving as the recombinase binding sites) flanking an 8 base pair core sequence (e.g., FIG. 1 of Sauer, B., Curr. Opin. Biotech. 5:521-527 (1994)). Other examples of recombination sites include attB, attP, attL, and attR sequences, and mutants, fragments, variants and derivatives thereof, which are recognized by the recombination protein λ Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent application Ser. No. 09/517,466, filed Mar. 2, 2000, and 09/732,914, filed Aug. 14, 2003, and in U.S. patent publication no. 2002-0007051-A1; Landy, Curr. Opin. Biotech. 3:699-707 (1993)).

Examples of recombinase cloning nucleic acids are in Gateway® systems (Invitrogen, California), which include at least one recombination site for cloning a desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

A recombination system useful for engineering yeast is outlined briefly. The system makes use of the ura3 gene (e.g., for S. cerevisiae and C. albicans, for example) or ura4 and ura5 genes (e.g., for S. pombe, for example) and toxicity of the nucleotide analogue 5-Fluoroorotic acid (5-FOA). The ura3 or ura4 and ura5 genes encode orotine-5′-monophosphate (OMP) dicarboxylase. Yeast with an active ura3 or ura4 and ura5 gene (phenotypically Ura+) convert 5-FOA to fluorodeoxyuridine, which is toxic to yeast cells. Yeast carrying a mutation in the appropriate gene(s) or having a knock out of the appropriate gene(s) can grow in the presence of 5-FOA, if the media is also supplemented with uracil.

A nucleic acid engineering construct can be made which may comprise the URA3 gene or cassette (for S. cerevisiae), flanked on either side by the same nucleotide sequence in the same orientation. The ura3 cassette comprises a promoter, the ura3 gene and a functional transcription terminator. Target sequences which direct the construct to a particular nucleic acid region of interest in the organism to be engineered are added such that the target sequences are adjacent to and abut the flanking sequences on either side of the ura3 cassette. Yeast can be transformed with the engineering construct and plated on minimal media without uracil. Colonies can be screened by PCR to determine those transformants that have the engineering construct inserted in the proper location in the genome. Checking insertion location prior to selecting for recombination of the ura3 cassette may reduce the number of incorrect clones carried through to later stages of the procedure. Correctly inserted transformants can then be replica plated on minimal media containing 5-FOA to select for recombination of the ura3 cassette out of the construct, leaving a disrupted gene and an identifiable footprint (e.g., nucleotide sequence) that can be use to verify the presence of the disrupted gene. The technique described is useful for disrupting or “knocking out” gene function, but also can be used to insert genes or constructs into a host organisms genome in a targeted, sequence specific manner.

In certain embodiments, a nucleic acid reagent includes one or more topoisomerase insertion sites. A topoisomerase insertion site is a defined nucleotide sequence recognized and bound by a site-specific topoisomerase. For example, the nucleotide sequence 5′-(C/T)CCTT-3′ is a topoisomerase recognition site bound specifically by most poxvirus topoisomerases, including vaccinia virus DNA topoisomerase I. After binding to the recognition sequence, the topoisomerase cleaves the strand at the 3′-most thymidine of the recognition site to produce a nucleotide sequence comprising 5′-(C/T)CCTT-PO4-TOPO, a complex of the topoisomerase covalently bound to the 3′ phosphate via a tyrosine in the topoisomerase (e.g., Shuman, J. Biol. Chem. 266:11372-11379, 1991; Sekiguchi and Shuman, Nucl. Acids Res. 22:5360-5365, 1994; U.S. Pat. No. 5,766,891; PCT/US95/16099; and PCT/US98/12372). In comparison, the nucleotide sequence 5′-GCAACTT-3′ is a topoisomerase recognition site for type IA E. coli topoisomerase III. An element to be inserted often is combined with topoisomerase-reacted template and thereby incorporated into the nucleic acid reagent (e.g., http address www.invitrogen.com/downloads/F-13512_Topo_Flyer.pdf; http address at www.invitrogen.com/content/sfs/brochures/710_{—}021849%20_B_TOPOCloning_bro.pdf; TOPO TA Cloning® Kit and Zero Blunt® TOPO® Cloning Kit product information).

A nucleic acid reagent sometimes contains one or more origin of replication (ORI) elements. In some embodiments, a template comprises two or more ORIs, where one functions efficiently in one organism (e.g., a bacterium) and another functions efficiently in another organism (e.g., a eukaryote, like yeast for example). In some embodiments, an ORI may function efficiently in one species (e.g., S. cerevisiae, for example) and another ORI may function efficiently in a different species (e.g., S. pombe, for example). A nucleic acid reagent also sometimes includes one or more transcription regulation sites.

A nucleic acid reagent can include one or more selection elements (e.g., elements for selection of the presence of the nucleic acid reagent, and not for activation of a promoter element which can be selectively regulated). Selection elements often are utilized using known processes to determine whether a nucleic acid reagent is included in a cell. In some embodiments, a nucleic acid reagent includes two or more selection elements, where one functions efficiently in one organism and another functions efficiently in another organism. Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

A nucleic acid reagent is of any form useful for in vivo transcription and/or translation. A nucleic acid sometimes is a plasmid, such as a supercoiled plasmid, sometimes is a yeast artificial chromosome (e.g., YAC), sometimes is a linear nucleic acid (e.g., a linear nucleic acid produced by PCR or by restriction digest), sometimes is single-stranded and sometimes is double-stranded. A nucleic acid reagent sometimes is prepared by an amplification process, such as a polymerase chain reaction (PCR) process or transcription-mediated amplification process (TMA). In TMA, two enzymes are used in an isothermal reaction to produce amplification products detected by light emission (see, e.g., Biochemistry 1996 Jun. 25; 35(25):8429-38 and http address www.devicelink.com/ivdt/archive/00/11/007.html). Standard PCR processes are known (e.g., U.S. Pat. Nos. 4,683,202; 4,683,195; 4,965,188; and 5,656,493), and generally are performed in cycles. Each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis.

In some embodiments, a nucleic acid reagent, protein reagent, protein fragment reagent or other reagent described herein is isolated or purified. The term “isolated” as used herein refers to material removed from its original environment (e.g., the natural environment if it is naturally occurring, or a host cell if expressed exogenously), and thus is altered “by the hand of man” from its original environment. The term “purified” as used herein with reference to molecules does not refer to absolute purity. Rather, “purified” refers to a substance in a composition that contains fewer substance species in the same class (e.g., nucleic acid or protein species) other than the substance of interest in comparison to the sample from which it originated. “Purified,” if a nucleic acid or protein for example, refers to a substance in a composition that contains fewer nucleic acid species or protein species other than the nucleic acid or protein of interest in comparison to the sample from which it originated. Sometimes, a protein or nucleic acid is “substantially pure,” indicating that the protein or nucleic acid represents at least 50% of protein or nucleic acid on a mass basis of the composition. Often, a substantially pure protein or nucleic acid is at least 75% on a mass basis of the composition, and sometimes at least 95% on a mass basis of the composition.

### Engineering and Alteration Methods

Methods and compositions (e.g., nucleic acid reagents) described herein can be used to generate engineered microorganisms. As noted above, the term “engineered microorganism” as used herein refers to a modified organism that includes one or more activities distinct from an activity present in a microorganism utilized as a starting point for modification (e.g., host microorganism or unmodified organism). Engineered microorganisms typically arise as a result of a genetic modification, usually introduced or selected for, by one of skill in the art using readily available techniques. Non-limiting examples of methods useful for generating an altered activity include, introducing a heterologus polynucleotide (e.g., nucleic acid or gene integration, also referred to as “knock in”), removing an endogenous polynucleotide, altering the sequence of an existing endogenous nucleotide sequence (e.g., site-directed mutagenesis), disruption of an existing endogenous nucleotide sequence (e.g., knock outs and transposon or insertion element mediated mutagenesis), selection for an altered activity where the selection causes a change in a naturally occurring activity that can be stably inherited (e.g., causes a change in a nucleotide sequence in the genome of the organism or in an epigenetic nucleic acid that is replicated and passed on to daughter cells), PCR-based mutagenesis, and the like. The term “mutagenesis” as used herein refers to any modification to a nucleic acid (e.g., nucleic acid reagent, or host chromosome, for example) that is subsequently used to generate a product in a host or modified organism. Non-limiting examples of mutagenesis include, deletion, insertion, substitution, rearrangement, point mutations, suppressor mutations and the like. Mutagenesis methods are known in the art and are readily available to the artisan. Non-limiting examples of mutagenesis methods are described herein and can also be found in Maniatis, T., E. F. Fritsch and J. Sambrook (1982) Molecular Cloning: a Laboratory Manual; Cold Spring Harbor Laboratory, Cold Spring Harbor, N.Y.

The term “genetic modification” as used herein refers to any suitable nucleic acid addition, removal or alteration that facilitates production of a target product (e.g., GGPP synthase activity, phytoene synthase activity, phytoene desaturase activity, for example), in an engineered microorganism. Genetic modifications include, without limitation, insertion of one or more nucleotides in a native nucleic acid of a host organism in one or more locations, deletion of one or more nucleotides in a native nucleic acid of a host organism in one or more locations, modification or substitution of one or more nucleotides in a native nucleic acid of a host organism in one or more locations, insertion of a non-native nucleic acid into a host organism (e.g., insertion of an autonomously replicating vector), and removal of a non-native nucleic acid in a host organism (e.g., removal of a vector).

The term “heterologus polynucleotide” as used herein refers to a nucleotide sequence not present in a host microorganism in some embodiments. In certain embodiments, a heterologus polynucleotide is present in a different amount (e.g., different copy number) than in a host microorganism, which can be accomplished, for example, by introducing more copies of a particular nucleotide sequence to a host microorganism (e.g., the particular nucleotide sequence may be in a nucleic acid autonomous of the host chromosome or may be inserted into a chromosome). A heterologus polynucleotide is from a different organism in some embodiments, and in certain embodiments, is from the same type of organism but from an outside source (e.g., a recombinant source).

The term “altered activity” as used herein refers to an activity in an engineered microorganism that is added or modified relative to the host microorganism (e.g., added, increased, reduced, inhibited or removed activity). An activity can be altered by introducing a genetic modification to a host microorganism that yields an engineered microorganism having added, increased, reduced, inhibited or removed activity.

An added activity often is an activity not detectable in a host microorganism. An increased activity generally is an activity detectable in a host microorganism that has been increased in an engineered microorganism. An activity can be increased to any suitable level for production of a target product (e.g., lycopene), including but not limited to less than 2-fold (e.g., about 10% increase to about 99% increase; about 20%, 30%, 40%, 50%, 60%, 70%, 80%, 90% increase), 2-fold, 3-fold, 4-fold, 5-fold, 6-fold, 7-fold, 8-fold, 9-fold, of 10-fold increase, or greater than about 10-fold increase. A reduced or inhibited activity generally is an activity detectable in a host microorganism that has been reduced or inhibited in an engineered microorganism. An activity can be reduced to undetectable levels in some embodiments, or detectable levels in certain embodiments. An activity can be decreased to any suitable level for production of a target product (e.g., lycopene), including but not limited to less than 2-fold (e.g., about 10% decrease to about 99% decrease; about 20%, 30%, 40%, 50%, 60%, 70%, 80%, 90% decrease), 2-fold, 3-fold, 4-fold, 5-fold, 6-fold, 7-fold, 8-fold, 9-fold, of 10-fold decrease, or greater than about 10-fold decrease.

An altered activity sometimes is an activity not detectable in a host organism and is added to an engineered organism. An altered activity also may be an activity detectable in a host organism and is increased in an engineered organism. An activity may be added or increased by increasing the number of copies of a polynucleotide that encodes a polypeptide having a target activity, in some embodiments. In certain embodiments an activity can be added or increased by inserting into a host microorganism a heterologus polynucleotide that encodes a polypeptide having the added activity. In certain embodiments, an activity can be added or increased by inserting into a host microorganism a heterologus polynucleotide that is (i) operably linked to another polynucleotide that encodes a polypeptide having the added activity, and (ii) up regulates production of the polynucleotide. Thus, an activity can be added or increased by inserting or modifying a regulatory polynucleotide operably linked to another polynucleotide that encodes a polypeptide having the target activity. In certain embodiments, an activity can be added or increased by subjecting a host microorganism to a selective environment and screening for microorganisms that have a detectable level of the target activity. Examples of a selective environment include, without limitation, a medium containing a substrate that a host organism can process and a medium lacking a substrate that a host organism can process.

An altered activity sometimes is an activity detectable in a host organism and is reduced, inhibited or removed (i.e., not detectable) in an engineered organism. An activity may be reduced or removed by decreasing the number of copies of a polynucleotide that encodes a polypeptide having a target activity, in some embodiments. In some embodiments, an activity can be reduced or removed by (i) inserting a polynucleotide within a polynucleotide that encodes a polypeptide having the target activity (disruptive insertion), and/or (ii) removing a portion of or all of a polynucleotide that encodes a polypeptide having the target activity (deletion or knock out, respectively). In certain embodiments, an activity can be reduced or removed by inserting into a host microorganism a heterologus polynucleotide that is (i) operably linked to another polynucleotide that encodes a polypeptide having the target activity, and (ii) down regulates production of the polynucleotide. Thus, an activity can be reduced or removed by inserting or modifying a regulatory polynucleotide operably linked to another polynucleotide that encodes a polypeptide having the target activity.

An activity also can be reduced or removed by (i) inhibiting a polynucleotide that encodes a polypeptide having the activity or (ii) inhibiting a polynucleotide operably linked to another polynucleotide that encodes a polypeptide having the activity. A polynucleotide can be inhibited by a suitable technique known in the art, such as by contacting an RNA encoded by the polynucleotide with a specific inhibitory RNA (e.g., RNAi, siRNA, ribozyme). An activity also can be reduced or removed by contacting a polypeptide having the activity with a molecule that specifically inhibits the activity (e.g., enzyme inhibitor, antibody). In certain embodiments, an activity can be reduced or removed by subjecting a host microorganism to a selective environment and screening for microorganisms that have a reduced level or removal of the target activity.

In some embodiments, an untranslated ribonucleic acid, or a cDNA can be used to reduce the expression of a particular activity or enzyme. For example, a microorganism can be engineered by genetic modification to express a nucleic acid reagent that reduces the expression of an activity by producing an RNA molecule that is partially or substantially homologous to a nucleotide sequence of interest which encodes the activity of interest. The RNA molecule can bind to the nucleotide sequence of interest and inhibit the nucleotide sequence from performing its natural function, in certain embodiments. In some embodiments, the RNA may alter the nucleotide sequence of interest which encodes the activity of interest in a manner that the nucleotide sequence of interest is no longer capable of performing its natural function (e.g., the action of a ribozyme for example).

In certain embodiments, nucleotide sequences sometimes are added to, modified or removed from one or more of the nucleic acid reagent elements, such as the promoter, 5′UTR, target sequence, or 3′UTR elements, to enhance, potentially enhance, reduce, or potentially reduce transcription and/or translation before or after such elements are incorporated in a nucleic acid reagent. In some embodiments, one or more of the following sequences may be modified or removed if they are present in a 5′UTR: a sequence that forms a stable secondary structure (e.g., quadruplex structure or stem loop stem structure (e.g., EMBL sequences X12949, AF274954, AF139980, AF152961, S95936, U194144, AF116649 or substantially identical sequences that form such stem loop stem structures)); a translation initiation codon upstream of the target nucleotide sequence start codon; a stop codon upstream of the target nucleotide sequence translation initiation codon; an ORF upstream of the target nucleotide sequence translation initiation codon; an iron responsive element (IRE) or like sequence; and a 5′ terminal oligopyrimidine tract (TOP, e.g., consisting of 5-15 pyrimidines adjacent to the cap). A translational enhancer sequence and/or an internal ribosome entry site (IRES) sometimes is inserted into a 5′UTR (e.g., EMBL nucleotide sequences J04513, X87949, M95825, M12783, AF025841, AF013263, AF006822, M17169, M13440, M22427, D14838 and M17446 and substantially identical nucleotide sequences).

An AU-rich element (ARE, e.g., AUUUA repeats) and/or splicing junction that follows a non-sense codon sometimes is removed from or modified in a 3′UTR. A polyadenosine tail sometimes is inserted into a 3′UTR if none is present, sometimes is removed if it is present, and adenosine moieties sometimes are added to or removed from a polyadenosine tail present in a 3′UTR. Thus, some embodiments are directed to a process comprising: determining whether any nucleotide sequences that increase, potentially increase, reduce or potentially reduce translation efficiency are present in the elements, and adding, removing or modifying one or more of such sequences if they are identified. Certain embodiments are directed to a process comprising: determining whether any nucleotide sequences that increase or potentially increase translation efficiency are not present in the elements, and incorporating such sequences into the nucleic acid reagent.

In some embodiments, an activity can be altered by modifying the nucleotide sequence of an ORF. An ORF sometimes is mutated or modified (for example, by point mutation, deletion mutation, insertion mutation, PCR based mutagenesis and the like) to alter, enhance or increase, reduce, substantially reduce or eliminate the activity of the encoded protein or peptide. The protein or peptide encoded by a modified ORF sometimes is produced in a lower amount or may not be produced at detectable levels, and in other embodiments, the product or protein encoded by the modified ORF is produced at a higher level (e.g., codons sometimes are modified so they are compatible with tRNA's preferentially used in the host organism or engineered organism). To determine the relative activity, the activity from the product of the mutated ORF (or cell containing it) can be compared to the activity of the product or protein encoded by the unmodified ORF (or cell containing it).

In some embodiments, an ORF nucleotide sequence sometimes is mutated or modified to alter the triplet nucleotide sequences used to encode amino acids (e.g., amino acid codon triplets, for example). Modification of the nucleotide sequence of an ORF to alter codon triplets sometimes is used to change the codon found in the original sequence to better match the preferred codon usage of the organism in which the ORF or nucleic acid reagent will be expressed. For example, the codon usage, and therefore the codon triplets encoded by a nucleotide sequence from bacteria may be different from the preferred codon usage in eukaryotes like yeast or plants. Preferred codon usage also may be different between bacterial species. In certain embodiments an ORF nucleotide sequences sometimes is modified to eliminate codon pairs and/or eliminate mRNA secondary structures that can cause pauses during translation of the mRNA encoded by the ORF nucleotide sequence. Translational pausing sometimes occurs when nucleic acid secondary structures exist in an mRNA, and sometimes occurs due to the presence of codon pairs that slow the rate of translation by causing ribosomes to pause. In some embodiments, the use of lower abundance codon triplets can reduce translational pausing due to a decrease in the pause time needed to load a charged tRNA into the ribosome translation machinery. Therefore, to increase transcriptional and translational efficiency in bacteria (e.g., where transcription and translation are concurrent, for example) or to increase translational efficiency in eukaryotes (e.g., where transcription and translation are functionally separated), the nucleotide sequence of a nucleotide sequence of interest can be altered to better suit the transcription and/or translational machinery of the host and/or genetically modified microorganism. In certain embodiment, slowing the rate of translation by the use of lower abundance codons, which slow or pause the ribosome, can lead to higher yields of the desired product due to an increase in correctly folded proteins and a reduction in the formation of inclusion bodies.

Codons can be altered and optimized according to the preferred usage by a given organism by determining the codon distribution of the nucleotide sequence donor organism and comparing the distribution of codons to the distribution of codons in the recipient or host organism. Techniques described herein (e.g., site directed mutagenesis and the like) can then be used to alter the codons accordingly. Comparisons of codon usage can be done by hand, or using nucleic acid analysis software commercially available to the artisan.

Modification of the nucleotide sequence of an ORF also can be used to correct codon triplet sequences that have diverged in different organisms. For example, certain yeast (e.g., C. tropicalis and C. maltosa) use the amino acid triplet CUG (e.g., CTG in the DNA sequence) to encode serine. CUG typically encodes leucine in most organisms. In order to maintain the correct amino acid in the resultant polypeptide or protein, the CUG codon must be altered to reflect the organism in which the nucleic acid reagent will be expressed. Thus, if an ORF from a bacterial donor is to be expressed in either Candida yeast strain mentioned above, the heterologus nucleotide sequence must first be altered or modified to the appropriate leucine codon. Therefore, in some embodiments, the nucleotide sequence of an ORF sometimes is altered or modified to correct for differences that have occurred in the evolution of the amino acid codon triplets between different organisms. In some embodiments, the nucleotide sequence can be left unchanged at a particular amino acid codon, if the amino acid encoded is a conservative or neutral change in amino acid when compared to the originally encoded amino acid.

In some embodiments, an activity can be altered by modifying translational regulation signals, like a stop codon for example. A stop codon at the end of an ORF sometimes is modified to another stop codon, such as an amber stop codon described above. In some embodiments, a stop codon is introduced within an ORF, sometimes by insertion or mutation of an existing codon. An ORF comprising a modified terminal stop codon and/or internal stop codon often is translated in a system comprising a suppressor tRNA that recognizes the stop codon. An ORF comprising a stop codon sometimes is translated in a system comprising a suppressor tRNA that incorporates an unnatural amino acid during translation of the target protein or target peptide. Methods for incorporating unnatural amino acids into a target protein or peptide are known, which include, for example, processes utilizing a heterologus tRNA/synthetase pair, where the tRNA recognizes an amber stop codon and is loaded with an unnatural amino acid (e.g., World Wide Web URL iupac.org/news/prize/2003/wang.pdf).

Depending on the portion of a nucleic acid reagent (e.g., Promoter, 5′ or 3′ UTR, ORI, ORF, and the like) chosen for alteration (e.g., by mutagenesis, introduction or deletion, for example) the modifications described above can alter a given activity by (i) increasing or decreasing feedback inhibition mechanisms, (ii) increasing or decreasing promoter initiation, (iii) increasing or decreasing translation initiation, (iv) increasing or decreasing translational efficiency, (v) modifying localization of peptides or products expressed from nucleic acid reagents described herein, or (vi) increasing or decreasing the copy number of a nucleotide sequence of interest, (vii) expression of an anti-sense RNA, RNAi, siRNA, ribozyme and the like. In some embodiments, alteration of a nucleic acid reagent or nucleotide sequence can alter a region involved in feedback inhibition (e.g., 5′ UTR, promoter and the like). A modification sometimes is made that can add or enhance binding of a feedback regulator and sometimes a modification is made that can reduce, inhibit or eliminate binding of a feedback regulator.

In certain embodiments, alteration of a nucleic acid reagent or nucleotide sequence can alter sequences involved in transcription initiation (e.g., promoters, 5′ UTR, and the like). A modification sometimes can be made that can enhance or increase initiation from an endogenous or heterologus promoter element. A modification sometimes can be made that removes or disrupts sequences that increase or enhance transcription initiation, resulting in a decrease or elimination of transcription from an endogenous or heterologus promoter element.

In some embodiments, alteration of a nucleic acid reagent or nucleotide sequence can alter sequences involved in translational initiation or translational efficiency (e.g., 5′ UTR, 3′ UTR, codon triplets of higher or lower abundance, translational terminator sequences and the like, for example). A modification sometimes can be made that can increase or decrease translational initiation, modifying a ribosome binding site for example. A modification sometimes can be made that can increase or decrease translational efficiency. Removing or adding sequences that form hairpins and changing codon triplets to a more or less preferred codon are non-limiting examples of genetic modifications that can be made to alter translation initiation and translation efficiency.

In certain embodiments, alteration of a nucleic acid reagent or nucleotide sequence can alter sequences involved in localization of peptides, proteins or other desired products (e.g., lycopene, for example). A modification sometimes can be made that can alter, add or remove sequences responsible for targeting a polypeptide, protein or product to an intracellular organelle, the periplasm, cellular membranes, or extracellularly. Transport of a heterologus product to a different intracellular space or extracellularly sometimes can reduce or eliminate the formation of inclusion bodies (e.g., insoluble aggregates of the desired product).

In some embodiments, alteration of a nucleic acid reagent or nucleotide sequence can alter sequences involved in increasing or decreasing the copy number of a nucleotide sequence of interest. A modification sometimes can be made that increases or decreases the number of copies of an ORF stably integrated into the genome of an organism or on an epigenetic nucleic acid reagent. Non-limiting examples of alterations that can increase the number of copies of a sequence of interest include, adding copies of the sequence of interest by duplication of regions in the genome (e.g., adding additional copies by recombination or by causing gene amplification of the host genome, for example), cloning additional copies of a sequence onto a nucleic acid reagent, or altering an ORI to increase the number of copies of an epigenetic nucleic acid reagent. Non-limiting examples of alterations that can decrease the number of copies of a sequence of interest include, removing copies of the sequence of interest by deletion or disruption of regions in the genome, removing additional copies of the sequence from epigenetic nucleic acid reagents, or altering an ORI to decrease the number of copies of an epigenetic nucleic acid reagent.

In certain embodiments, increasing or decreasing the expression of a nucleotide sequence of interest can also be accomplished by altering, adding or removing sequences involved in the expression of an anti-sense RNA, RNAi, siRNA, ribozyme and the like. The methods described above can be used to modify expression of anti-sense RNA, RNAi, siRNA, ribozyme and the like.

Engineered microorganisms can be prepared by altering, introducing or removing nucleotide sequences in the host genome or in stably maintained epigenetic nucleic acid reagents, as noted above. The nucleic acid reagents use to alter, introduce or remove nucleotide sequences in the host genome or epigenetic nucleic acids can be prepared using the methods described herein or available to the artisan.

Nucleotide sequences having a desired activity can be isolated from cells of a suitable organism using lysis and nucleic acid purification procedures available in Maniatis, T., E. F. Fritsch and J. Sambrook (1982) Molecular Cloning: a Laboratory Manual; Cold Spring Harbor Laboratory, Cold Spring Harbor, N.Y. or with commercially available cell lysis and DNA purification reagents and kits. In some embodiments, nucleic acids used to engineer microorganisms can be provided for conducting methods described herein after processing of the organism containing the nucleic acid. For example, the nucleic acid of interest may be extracted, isolated, purified or amplified from a sample (e.g., from an organism of interest or culture containing a plurality of organisms of interest, like yeast or bacteria for example). The term “isolated” as used herein refers to nucleic acid removed from its original environment (e.g., the natural environment if it is naturally occurring, or a host cell if expressed exogenously), and thus is altered “by the hand of man” from its original environment. An isolated nucleic acid generally is provided with fewer non-nucleic acid components (e.g., protein, lipid) than the amount of components present in a source sample. A composition comprising isolated sample nucleic acid can be substantially isolated (e.g., about 90%, 91%, 92%, 93%, 94%, 95%, 96%, 97%, 98%, 99% or greater than 99% free of non-nucleic acid components). The term “purified” as used herein refers to sample nucleic acid provided that contains fewer nucleic acid species than in the sample source from which the sample nucleic acid is derived. A composition comprising sample nucleic acid may be substantially purified (e.g., about 90%, 91%, 92%, 93%, 94%, 95%, 96%, 97%, 98%, 99% or greater than 99% free of other nucleic acid species). The term “amplified” as used herein refers to subjecting nucleic acid of a cell, organism or sample to a process that linearly or exponentially generates amplicon nucleic acids having the same or substantially the same nucleotide sequence as the nucleotide sequence of the nucleic acid in the sample, or portion thereof. As noted above, the nucleic acids used to prepare nucleic acid reagents as described herein can be subjected to fragmentation or cleavage.

Amplification of nucleic acids is sometimes necessary when dealing with organisms that are difficult to culture. Where amplification may be desired, any suitable amplification technique can be utilized. Non-limiting examples of methods for amplification of polynucleotides include, polymerase chain reaction (PCR); ligation amplification (or ligase chain reaction (LCR)); amplification methods based on the use of Q-beta replicase or template-dependent polymerase (see US Patent Publication Number US20050287592); helicase-dependant isothermal amplification (Vincent et al., “Helicase-dependent isothermal DNA amplification”. EMBO reports 5 (8): 795-800 (2004)); strand displacement amplification (SDA); thermophilic SDA nucleotide sequence based amplification (3SR or NASBA) and transcription-associated amplification (TAA). Non-limiting examples of PCR amplification methods include standard PCR, AFLP-PCR, Allele-specific PCR, Alu-PCR, Asymmetric PCR, Colony PCR, Hot start PCR, Inverse PCR (IPCR), In situ PCR (ISH), Intersequence-specific PCR (ISSR-PCR), Long PCR, Multiplex PCR, Nested PCR, Quantitative PCR, Reverse Transcriptase PCR (RT-PCR), Real Time PCR, Single cell PCR, Solid phase PCR, combinations thereof, and the like. Reagents and hardware for conducting PCR are commercially available.

Protocols for conducting the various type of PCR listed above are readily available to the artisan. PCR conditions can be dependent upon primer sequences, target abundance, and the desired amount of amplification, and therefore, one of skill in the art may choose from a number of PCR protocols available (see, e.g., U.S. Pat. Nos. 4,683,195 and 4,683,202; and PCR Protocols: A Guide to Methods and Applications, Innis et al., eds, 1990. PCR often is carried out as an automated process with a thermostable enzyme. In this process, the temperature of the reaction mixture is cycled through a denaturing region, a primer-annealing region, and an extension reaction region automatically. Machines specifically adapted for this purpose are commercially available. A non-limiting example of a PCR protocol that may be suitable for embodiments described herein is, treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Additional PCR protocols are described in the example section. Multiple cycles frequently are performed using a commercially available thermal cycler. Suitable isothermal amplification processes known and selected by the person of ordinary skill in the art also may be applied, in certain embodiments. In some embodiments, nucleic acids encoding polypeptides with a desired activity can be isolated by amplifying the desired sequence from an organism having the desired activity using oligonucleotides or primers designed based on sequences described herein

Amplified, isolated and/or purified nucleic acids can be cloned into the recombinant DNA vectors described in Figures herein or into suitable commercially available recombinant DNA vectors. Cloning of nucleotide sequences of interest into recombinant DNA vectors can facilitate further manipulations of the nucleic acids for preparation of nucleic acid reagents, (e.g., alteration of nucleotide sequences by mutagenesis, homologous recombination, amplification and the like, for example). Standard cloning procedures (e.g., enzymic digestion, ligation, and the like) are readily available to the artisan and can be found in Maniatis, T., E. F. Fritsch and J. Sambrook (1982) Molecular Cloning: a Laboratory Manual; Cold Spring Harbor Laboratory, Cold Spring Harbor, N.Y.

In some embodiments, nucleotide sequences prepared by isolation or amplification can be used, without any further modification, to add an activity to a microorganism and thereby generate a genetically modified or engineered microorganism. In certain embodiments, nucleotide sequences prepared by isolation or amplification can be genetically modified to alter (e.g., increase or decrease, for example) a desired activity. In some embodiments, nucleic acids, used to add an activity to an organism, sometimes are genetically modified to optimize the heterologus polynucleotide sequence encoding the desired activity (e.g., polypeptide or protein, for example). The term “optimize” as used herein can refer to alteration to increase or enhance expression by preferred codon usage. The term optimize can also refer to modifications to the amino acid sequence to increase the activity of a polypeptide or protein, such that the activity exhibits a higher catalytic activity as compared to the “natural” version of the polypeptide or protein.

Nucleotide sequences of interest can be genetically modified using methods known in the art. Mutagenesis techniques are particularly useful for small scale (e.g., 1, 2, 5, 10 or more nucleotides) or large scale (e.g., 50, 100, 150, 200, 500, or more nucleotides) genetic modification. Mutagenesis allows the artisan to alter the genetic information of an organism in a stable manner, either naturally (e.g., isolation using selection and screening) or experimentally by the use of chemicals, radiation or inaccurate DNA replication (e.g., PCR mutagenesis). In some embodiments, genetic modification can be performed by whole scale synthetic synthesis of nucleic acids, using a native nucleotide sequence as the reference sequence, and modifying nucleotides that can result in the desired alteration of activity. Mutagenesis methods sometimes are specific or targeted to specific regions or nucleotides (e.g., site-directed mutagenesis, PCR-based site-directed mutagenesis, and in vitro mutagenesis techniques such as transplacement and in vivo oligonucleotide site-directed mutagenesis, for example). Mutagenesis methods sometimes are non-specific or random with respect to the placement of genetic modifications (e.g., chemical mutagenesis, insertion element (e.g., insertion or transposon elements) and inaccurate PCR based methods, for example).

Site directed mutagenesis is a procedure in which a specific nucleotide or specific nucleotides in a DNA molecule are mutated or altered. Site directed mutagenesis typically is performed using a nucleotide sequence of interest cloned into a circular plasmid vector. Site-directed mutagenesis requires that the wild type sequence be known and used a platform for the genetic alteration. Site-directed mutagenesis sometimes is referred to as oligonucleotide-directed mutagenesis because the technique can be performed using oligonucleotides which have the desired genetic modification incorporated into the complement a nucleotide sequence of interest. The wild type sequence and the altered nucleotide are allowed to hybridize and the hybridized nucleic acids are extended and replicated using a DNA polymerase. The double stranded nucleic acids are introduced into a host (e.g., E. coli, for example) and further rounds of replication are carried out in vivo. The transformed cells carrying the mutated nucleotide sequence are then selected and/or screened for those cells carrying the correctly mutagenized sequence. Cassette mutagenesis and PCR-based site-directed mutagenesis are further modifications of the site-directed mutagenesis technique. Site-directed mutagenesis can also be performed in vivo (e.g., transplacement “pop-in pop-out”, In vivo site-directed mutagenesis with synthetic oligonucleotides and the like, for example).

PCR-based mutagenesis can be performed using PCR with oligonucleotide primers that contain the desired mutation or mutations. The technique functions in a manner similar to standard site-directed mutagenesis, with the exception that a thermocycler and PCR conditions are used to replace replication and selection of the clones in a microorganism host. As PCR-based mutagenesis also uses a circular plasmid vector, the amplified fragment (e.g., linear nucleic acid molecule) containing the incorporated genetic modifications can be separated from the plasmid containing the template sequence after a sufficient number of rounds of thermocycler amplification, using standard electrophorectic procedures. A modification of this method uses linear amplification methods and a pair of mutagenic primers that amplify the entire plasmid. The procedure takes advantage of the E. coli Dam methylase system which causes DNA replicated in vivo to be sensitive to the restriction endonucleases DpnI. PCR synthesized DNA is not methylated and is therefore resistant to DpnI. This approach allows the template plasmid to be digested, leaving the genetically modified, PCR synthesized plasmids to be isolated and transformed into a host bacteria for DNA repair and replication, thereby facilitating subsequent cloning and identification steps. A certain amount of randomness can be added to PCR-based sited directed mutagenesis by using partially degenerate primers.

Recombination sometimes can be used as a tool for mutagenesis. Homologous recombination allows the artisan to specifically target regions of known sequence for insertion of heterologus nucleotide sequences using the host organisms natural DNA replication and repair enzymes. Homologous recombination methods sometimes are referred to as “pop in pop out” mutagenesis, transplacement, knock out mutagenesis or knock in mutagenesis. Integration of a nucleotide sequence into a host genome is a single cross over event, which inserts the entire nucleic acid reagent (e.g., pop in). A second cross over event excises all but a portion of the nucleic acid reagent, leaving behind a heterologus sequence, often referred to as a “footprint” (e.g., pop out). Mutagenesis by insertion (e.g., knock in) or by double recombination leaving behind a disrupting heterologus nucleic acid (e.g., knock out) both server to disrupt or “knock out” the function of the gene or nucleotide sequence in which insertion occurs. By combining selectable markers and/or auxotrophic markers with nucleic acid reagents designed to provide the appropriate nucleic acid target sequences, the artisan can target a selectable nucleic acid reagent to a specific region, and then select for recombination events that “pop out” a portion of the inserted (e.g., “pop in”) nucleic acid reagent.

Such methods take advantage of nucleic acid reagents that have been specifically designed with known target nucleotide sequences at or near a nucleic acid or genomic region of interest. Popping out typically leaves a “foot print” of left over sequences that remain after the recombination event. The left over sequence can disrupt a gene and thereby reduce or eliminate expression of that gene. In some embodiments, the method can be used to insert sequences, upstream or downstream of genes that can result in an enhancement or reduction in expression of the gene. In certain embodiments, new genes can be introduced into the genome of a host organism using similar recombination or “pop in” methods. An example of a yeast recombination system using the ura3 gene and 5-FOA were described briefly above and further detail is presented herein.

A method for modification is described in Alani et al., “A method for gene disruption that allows repeated use of URA3 selection in the construction of multiply disrupted yeast strains”, Genetics 116(4):541-545 August 1987. The original method uses a Ura3 cassette with 1000 base pairs (bp) of the same nucleotide sequence cloned in the same orientation on either side of the URA3 cassette. Targeting sequences of about 50 bp are added to each side of the construct. The double stranded targeting sequences are complementary to sequences in the genome of the host organism. The targeting sequences allow site-specific recombination in a region of interest. The modification of the original technique replaces the two 1000 bp sequence direct repeats with two 200 bp direct repeats. The modified method also uses 50 bp targeting sequences. The modification reduces or eliminates recombination of a second knock out into the 1000 bp repeat left behind in a first mutagenesis, therefore allowing multiply knocked out yeast. Additionally, the 200 bp sequences used herein are uniquely designed, self-assembling sequences that leave behind identifiable footprints. The technique used to design the sequences incorporate design features such as low identity to the yeast genome, and low identity to each other. Therefore a library of the self-assembling sequences can be generated to allow multiple knockouts in the same organism, while reducing or eliminating the potential for integration into a previous knockout.

As noted above, the URA3 cassette makes use of the toxicity of 5-FOA in yeast carrying a functional URA3 gene. Uracil synthesis deficient yeast are transformed with the modified URA3 cassette, using standard yeast transformation protocols, and the transformed cells are plated on minimal media minus uracil. In some embodiments, PCR can be used to verify correct insertion into the region of interest in the host genome, and certain embodiments the PCR step can be omitted. Inclusion of the PCR step can reduce the number of transformants that need to be counter selected to “pop out” the URA3 cassette. The transformants (e.g., all or the ones determined to be correct by PCR, for example) can then be counter-selected on media containing 5-FOA, which will select for recombination out (e.g., popping out) of the URA3 cassette, thus rendering the yeast ura3 deficient again, and resistant to 5-FOA toxicity. Targeting sequences used to direct recombination events to specific regions are presented herein. A modification of the method described above can be used to integrate genes in to the chromosome, where after recombination a functional gene is left in the chromosome next to the 200 bp footprint.

In some embodiments, other auxotrophic or dominant selection markers can be used in place of URA3 (e.g., an auxotrophic selectable marker), with the appropriate change in selection media and selection agents. Auxotrophic selectable markers are used in strains deficient for synthesis of a required biological molecule (e.g., amino acid or nucleoside, for example). Non-limiting examples of additional auxotrophic markers include; HIS3, TRP1, LEU2, LEU2-d, and LYS2. Certain auxotrophic markers (e.g., URA3 and LYS2) allow counter selection to select for the second recombination event that pops out all but one of the direct repeats of the recombination construct. HIS3 encodes an activity involved in histidine synthesis. TRP1 encodes an activity involved in tryptophan synthesis. LEU2 encodes an activity involved in leucine synthesis. LEU2-d is a low expression version of LEU2 that selects for increased copy number (e.g., gene or plasmid copy number, for example) to allow survival on minimal media without leucine. LYS2 encodes an activity involved in lysine synthesis, and allows counter selection for recombination out of the LYS2 gene using alpha-amino adipate (α-amino adipate).

Dominant selectable markers are useful because they also allow industrial and/or prototrophic strains to be used for genetic manipulations. Additionally, dominant selectable markers provide the advantage that rich medium can be used for plating and culture growth, and thus growth rates are markedly increased. Non-limiting examples of dominant selectable markers include; Tn903 kan^{r}, Cm^{r}, Hyg^{r}, CUP1, and DHFR. Tn903 kan^{r} encodes an activity involved in kanamycin antibiotic resistance (e.g., typically neomycin phosphotransferase II or NPTII, for example). Cm^{r} encodes an activity involved in chloramphenicol antibiotic resistance (e.g., typically chloramphenicol acetyl transferase or CAT, for example). Hyg^{r} encodes an activity involved in hygromycin resistance by phosphorylation of hygromycin B (e.g., hygromycin phosphotransferase, or HPT). CUP1 encodes an activity involved in resistance to heavy metal (e.g., copper, for example) toxicity. DHFR encodes a dihydrofolate reductase activity which confers resistance to methotrexate and sulfanilamde compounds.

In contrast to site-directed or specific mutagenesis, random mutagenesis does not require any sequence information and can be accomplished by a number of widely different methods. Random mutagenesis often is used to generate mutant libraries that can be used to screen for the desired genotype or phenotype. Non-limiting examples of random mutagenesis include; chemical mutagenesis, UV-induced mutagenesis, insertion element or transposon-mediated mutagenesis, DNA shuffling, error-prone PCR mutagenesis, and the like.

Chemical mutagenesis often involves chemicals like ethyl methanesulfonate (EMS), nitrous acid, mitomycin C, N-methyl-N-nitrosourea (MNU), diepoxybutane (DEB), 1, 2, 7, 8-diepoxyoctane (DEO), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl-2-chloroethyl]-aminopropylamino)-acridinedihydrochloride (ICR-170), 2-amino purine (2AP), and hydroxylamine (HA), provided herein as non-limiting examples. These chemicals can cause base-pair substitutions, frameshift mutations, deletions, transversion mutations, transition mutations, incorrect replication, and the like. In some embodiments, the mutagenesis can be carried out in vivo. Sometimes the mutagenic process involves the use of the host organisms DNA replication and repair mechanisms to incorporate and replicate the mutagenized base or bases.

Another type of chemical mutagenesis involves the use of base-analogs. The use of base-analogs cause incorrect base pairing which in the following round of replication is corrected to a mismatched nucleotide when compared to the starting sequence. Base analog mutagenesis introduces a small amount of non-randomness to random mutagenesis, because specific base analogs can be chose which can be incorporated at certain nucleotides in the starting sequence. Correction of the mispairing typically yields a known substitution. For example, Bromo-deoxyuridine (BrdU) can be incorporated into DNA and replaces T in the sequence. The host DNA repair and replication machinery can sometime correct the defect, but sometimes will mispair the BrdU with a G. The next round of replication then causes a G-C transversion from the original A-T in the native sequence.

Ultra violet (UV) induced mutagenesis is caused by the formation of thymidine dimers when UV light irradiates chemical bonds between two adjacent thymine residues. Excision repair mechanism of the host organism correct the lesion in the DNA, but occasionally the lesion is incorrectly repaired typically resulting in a C to T transition.

Insertion element or transposon-mediated mutagenesis makes use of naturally occurring or modified naturally occurring mobile genetic elements. Transposons often encode accessory activities in addition to the activities necessary for transposition (e.g., movement using a transposase activity, for example). In many examples, transposon accessory activities are antibiotic resistance markers (e.g., see Tn903 kan^{r} described above, for example). Insertion elements typically only encode the activities necessary for movement of the nucleotide sequence. Insertion element and transposon mediated mutagenesis often can occur randomly, however specific target sequences are known for some transposons. Mobile genetic elements like IS elements or Transposons (Tn) often have inverted repeats, direct repeats or both inverted and direct repeats flanking the region coding for the transposition genes. Recombination events catalyzed by the transposase cause the element to remove itself from the genome and move to a new location, leaving behind a portion of an inverted or direct repeat. Classic examples of transposons are the “mobile genetic elements” discovered in maize. Transposon mutagenesis kits are commercially available which are designed to leave behind a 5 codon insert (e.g., Mutation Generation System kit, Finnzymes, World Wide Web URL finnzymes.us, for example). This allows the artisan to identify the insertion site, without fully disrupting the function of most genes.

DNA shuffling is a method which uses DNA fragments from members of a mutant library and reshuffles the fragments randomly to generate new mutant sequence combinations. The fragments are typically generated using DNaseI, followed by random annealing and re-joining using self priming PCR. The DNA overhanging ends, from annealing of random fragments, provide “primer” sequences for the PCR process. Shuffling can be applied to libraries generated by any of the above mutagenesis methods.

Error prone PCR and its derivative rolling circle error prone PCR uses increased magnesium and manganese concentrations in conjunction with limiting amounts of one or two nucleotides to reduce the fidelity of the Taq polymerase. The error rate can be as high as 2% under appropriate conditions, when the resultant mutant sequence is compared to the wild type starting sequence. After amplification, the library of mutant coding sequences must be cloned into a suitable plasmid. Although point mutations are the most common types of mutation in error prone PCR, deletions and frameshift mutations are also possible. There are a number of commercial error-prone PCR kits available, including those from Stratagene and Clontech (e.g., World Wide Web URL strategene.com and World Wide Web URL clontech.com, respectively, for example). Rolling circle error-prone PCR is a variant of error-prone PCR in which wild-type sequence is first cloned into a plasmid, the whole plasmid is then amplified under error-prone conditions.

As noted above, organisms with altered activities can also be isolated using genetic selection and screening of organisms challenged on selective media or by identifying naturally occurring variants from unique environments. For example, 2-Deoxy-D-glucose is a toxic glucose analog. Growth of yeast on this substance yields mutants that are glucose-deregulated. A number of mutants have been isolated using 2-Deoxy-D-glucose including transport mutants, and mutants that ferment glucose and galactose simultaneously instead of glucose first then galactose when glucose is depleted. Similar techniques have been used to isolate mutant microorganisms that can metabolize plastics (e.g., from landfills), petrochemicals (e.g., from oil spills), and the like, either in a laboratory setting or from unique environments.

Similar methods can be used to isolate naturally occurring mutations in a desired activity when the activity exists at a relatively low or nearly undetectable level in the organism of choice, in some embodiments. The method generally consists of growing the organism to a specific density in liquid culture, concentrating the cells, and plating the cells on various concentrations of the substance to which an increase in metabolic activity is desired. The cells are incubated at a moderate growth temperature, for 5 to 10 days. To enhance the selection process, the plates can be stored for another 5 to 10 days at a low temperature. The low temperature sometimes can allow strains that have gained or increased an activity to continue growing while other strains are inhibited for growth at the low temperature. Following the initial selection and secondary growth at low temperature, the plates can be replica plated on higher or lower concentrations of the selection substance to further select for the desired activity.

A native, heterologus or mutagenized polynucleotide can be introduced into a nucleic acid reagent for introduction into a host organism, thereby generating an engineered microorganism. Standard recombinant DNA techniques (restriction enzyme digests, ligation, and the like) can be used by the artisan to combine the mutagenized nucleic acid of interest into a suitable nucleic acid reagent capable of (i) being stably maintained by selection in the host organism, or (ii) being integrating into the genome of the host organism. As noted above, sometimes nucleic acid reagents comprise two replication origins to allow the same nucleic acid reagent to be manipulated in bacterial before final introduction of the final product into the host organism (e.g., yeast or fungus for example). Standard molecular biology and recombinant DNA methods available to one of skill in the art can be found in Maniatis, T., E. F. Fritsch and J. Sambrook (1982) Molecular Cloning: a Laboratory Manual; Cold Spring Harbor Laboratory, Cold Spring Harbor, N.Y.

Nucleic acid reagents can be introduced into microorganisms using various techniques. Non-limiting examples of methods used to introduce heterologus nucleic acids into various organisms include; transformation, transfection, transduction, electroporation, ultrasound-mediated transformation, particle bombardment and the like. In some instances the addition of carrier molecules (e.g., bis-benzimdazolyl compounds, for example, see U.S. Pat. No. 5,595,899) can increase the uptake of DNA in cells typically though to be difficult to transform by conventional methods. Conventional methods of transformation are readily available to the artisan and can be found in Maniatis, T., E. F. Fritsch and J. Sambrook (1982) Molecular Cloning: a Laboratory Manual; Cold Spring Harbor Laboratory, Cold Spring Harbor, N.Y.

### Culture, Production and Process Methods

Engineered microorganisms often are cultured under conditions that optimize yield of a target molecule. A non-limiting example of such a target molecule is lycopene. Culture conditions often can alter (e.g., add, optimize, reduce or eliminate, for example) activity of one or more of the following activities: GGPP synthase activity, phytoene synthase activity, phytoene desaturase activity. In general, conditions that may be optimized include the type and amount of carbon source, the type and amount of nitrogen source, the carbon-to-nitrogen ratio, the oxygen level, growth temperature, pH, length of the biomass production phase, length of target product accumulation phase, and time of cell harvest.

The term “fermentation conditions” as used herein refers to any culture conditions suitable for maintaining a microorganism (e.g., in a static or proliferative state). Fermentation conditions can include several parameters, including without limitation, temperature, oxygen content, nutrient content (e.g., glucose content), pH, agitation level (e.g., revolutions per minute), gas flow rate (e.g., air, oxygen, nitrogen gas), redox potential, cell density (e.g., optical density), cell viability and the like. A change in fermentation conditions (e.g., switching fermentation conditions) is an alteration, modification or shift of one or more fermentation parameters. For example, one can change fermentation conditions by increasing or decreasing temperature, increasing or decreasing pH (e.g., adding or removing an acid, a base or carbon dioxide), increasing or decreasing oxygen content (e.g., introducing air, oxygen, carbon dioxide, nitrogen) and/or adding or removing a nutrient (e.g., one or more sugars or sources of sugar, biomass, vitamin and the like), or combinations of the foregoing. Examples of fermentation conditions are described herein. Aerobic conditions often comprise greater than about 50% dissolved oxygen (e.g., about 52%, 54%, 56%, 58%, 60%, 62%, 64%, 66%, 68%, 70%, 72%, 74%, 76%, 78%, 80%, 82%, 84%, 86%, 88%, 90%, 91%, 92%, 93%, 94%, 95%, 96%, 97%, 98% or 99%, or greater than any one of the foregoing). Anaerobic conditions often comprise less than about 50% dissolved oxygen (e.g., about 1%, 2%, 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, or less than any one of the foregoing).

Culture media generally contain a suitable carbon source. Carbon sources may include, but are not limited to, monosaccharides (e.g., glucose, fructose, xylose), disaccharides (e.g., lactose, sucrose), oligosaccharides, polysaccharides (e.g., starch, cellulose, hemicellulose, other lignocellulosic materials or mixtures thereof), sugar alcohols (e.g., glycerol), and renewable feedstocks (e.g., cheese whey permeate, cornsteep liquor, sugar beet molasses, barley malt). Carbon sources also can be selected from one or more of the following non-limiting examples: linear or branched alkanes (e.g., hexane), linear or branched alcohols (e.g., hexanol), fatty acids (e.g., about 10 carbons to about 22 carbons), esters of fatty acids, monoglycerides, diglycerides, triglycerides, phospholipids and various commercial sources of fatty acids including vegetable oils (e.g., soybean oil) and animal fats. A carbon source may include one-carbon sources (e.g., carbon dioxide, methanol, formaldehyde, formate and carbon-containing amines) from which metabolic conversion into key biochemical intermediates can occur. It is expected that the source of carbon utilized may encompass a wide variety of carbon-containing sources and will only be limited by the choice of the engineered microorganism(s).

Nitrogen may be supplied from an inorganic (e.g., (NH.sub.4).sub.2SO.sub.4) or organic source (e.g., urea or glutamate). In addition to appropriate carbon and nitrogen sources, culture media also can contain suitable minerals, salts, cofactors, buffers, vitamins, metal ions (e.g., Mn.sup.+2, Co.sup.+2, Zn.sup.+2, Mg.sup.+2) and other components suitable for culture of microorganisms.

Engineered microorganisms sometimes are cultured in complex media (e.g., yeast extract-peptone-dextrose broth (YPD)). In some embodiments, engineered microorganisms are cultured in a defined minimal media that lacks a component necessary for growth and thereby forces selection of a desired expression cassette (e.g., Yeast Nitrogen Base (DIFCO Laboratories, Detroit, Mich.)). Culture media in some embodiments are common commercially prepared media, such as Yeast Nitrogen Base (DIFCO Laboratories, Detroit, Mich.). Other defined or synthetic growth media may also be used and the appropriate medium for growth of the particular microorganism are known.

A variety of host organisms can be selected for the production of engineered microorganisms. Non-limiting examples include bacteria algae, yeast and fungi. In specific embodiments, yeast are cultured in YPD media (10 g/L Bacto Yeast Extract, 20 g/L Bacto Peptone, and 20 g/L Dextrose). Filamentous fungi, in particular embodiments, are grown in CM (Complete Medium) containing 10 g/L Dextrose, 2 g/L Bacto Peptone, 1 g/L Bacto Yeast Extract, 1 g/L Casamino acids, 50 mL/L 20× Nitrate Salts (120 g/L NaNO₃, 10.4 g/L KCl, 10.4 g/L MgSO₄.7H₂O), 1 mL/L 1000× Trace Elements (22 g/L ZnSO₄.7H₂O, 11 g/L H₃BO₃, 5 g/L MnCl₂.7H₂O, 5 g/L FeSO₄.7H₂O, 1.7 g/L CoCl₂.6H₂O, 1.6 g/L CuSO₄.5H₂O, 1.5 g/L Na₂MoO₄.2H₂O, and 50 g/L Na₄EDTA), and 1 mL/L Vitamin Solution (100 mg each of Biotin, pyridoxine, thiamine, riboflavin, p-aminobenzoic acid, and nicotinic acid in 100 mL water).

A suitable pH range for the fermentation often is between about pH 4.0 to about pH 8.0, where a pH in the range of about pH 5.5 to about pH 7.0 sometimes is utilized for initial culture conditions. Culturing may be conducted under aerobic or anaerobic conditions, where microaerobic conditions sometimes are maintained. A two-stage process may be utilized, where one stage promotes microorganism proliferation and another state promotes production of target molecule. In a two-stage process, the first stage may be conducted under aerobic conditions (e.g., introduction of air and/or oxygen) and the second stage may be conducted under anaerobic conditions (e.g., air or oxygen are not introduced to the culture conditions).

A variety of fermentation processes may be applied for commercial biological production of a target product. In some embodiments, commercial production of a target product from a recombinant microbial host is conducted using a batch, fed-batch or continuous fermentation process, for example.

A batch fermentation process often is a closed system where the media composition is fixed at the beginning of the process and not subject to further additions beyond those required for maintenance of pH and oxygen level during the process. At the beginning of the culturing process the media is inoculated with the desired organism and growth or metabolic activity is permitted to occur without adding additional sources (i.e., carbon and nitrogen sources) to the medium. In batch processes the metabolite and biomass compositions of the system change constantly up to the time the culture is terminated. In a typical batch process, cells proceed through a static lag phase to a high-growth log phase and finally to a stationary phase, wherein the growth rate is diminished or halted. Left untreated, cells in the stationary phase will eventually die.

A variation of the standard batch process is the fed-batch process, where the carbon source is continually added to the fermentor over the course of the fermentation process. Fed-batch processes are useful when catabolite repression is apt to inhibit the metabolism of the cells or where it is desirable to have limited amounts of carbon source in the media at any one time. Measurement of the carbon source concentration in fed-batch systems may be estimated on the basis of the changes of measurable factors such as pH, dissolved oxygen and the partial pressure of waste gases (e.g., CO.sub.2).

Batch and fed-batch culturing methods are known in the art. Examples of such methods may be found in Thomas D. Brock in Biotechnology: A Textbook of Industrial Microbiology, 2.sup.nd ed., (1989) Sinauer Associates Sunderland, Mass. and Deshpande, Mukund V., Appl. Biochem. Biotechnol., 36:227 (1992).

In continuous fermentation process a defined media often is continuously added to a bioreactor while an equal amount of culture volume is removed simultaneously for product recovery. Continuous cultures generally maintain cells in the log phase of growth at a constant cell density. Continuous or semi-continuous culture methods permit the modulation of one factor or any number of factors that affect cell growth or end product concentration. For example, an approach may limit the carbon source and allow all other parameters to moderate metabolism. In some systems, a number of factors affecting growth may be altered continuously while the cell concentration, measured by media turbidity, is kept constant. Continuous systems often maintain steady state growth and thus the cell growth rate often is balanced against cell loss due to media being drawn off the culture. Methods of modulating nutrients and growth factors for continuous culture processes, as well as techniques for maximizing the rate of product formation, are known and a variety of methods are detailed by Brock, supra.

In various embodiments the desired product (e.g., lycopene or ethanol, for example) may be purified from the culture media or extracted from the engineered microorganisms. Culture media may be tested for concentration or the desired product and drawn off when the concentration reaches a predetermined level. Detection methods for many compounds produced by metabolic pathways are known in the art, including but not limited to the use of a growth of the host organism on media that presents a chromogenic change when lycopene or other intermediate beta-carotenoids are produced, western blot analysis, spectrometric analysis and the like, for example.

A target product sometimes is retained within an engineered microorganism after a culture process is completed, and in certain embodiments, the target product is secreted out of the microorganism into the culture medium. For the latter embodiments, (i) culture media may be drawn from the culture system and fresh medium may be supplemented, and/or (ii) target product may be extracted from the culture media during or after the culture process is completed. Engineered microorganisms may be cultured on or in solid, semi-solid or liquid media. In some embodiments media is drained from cells adhering to a plate. In certain embodiments, a liquid-cell mixture is centrifuged at a speed sufficient to pellet the cells but not disrupt the cells and allow extraction of the media, as known in the art. The cells may then be resuspended in fresh media. Target product may be purified from culture media according to methods known in the art.

In certain embodiments, target product is extracted from the cultured engineered microorganisms. The microorganism cells may be concentrated through centrifugation at speed sufficient to shear the cell membranes. In some embodiments, the cells may be physically disrupted (e.g., shear force, sonication) or chemically disrupted (e.g., contacted with detergent or other lysing agent). The phases may be separated by centrifugation or other method known in the art and target product may be isolated according to known methods.

Commercial grade target product sometimes is provided in substantially pure form (e.g., 90% pure or greater, 95% pure or greater, 99% pure or greater or 99.5% pure or greater). In some embodiments, target product may be modified into any one of a number of downstream products.

Target product may be provided within cultured microbes containing target product, and cultured microbes may be supplied fresh or frozen in a liquid media or dried. Fresh or frozen microbes may be contained in appropriate moisture-proof containers that may also be temperature controlled as necessary. Target product sometimes is provided in culture medium that is substantially cell-free. In some embodiments target product or modified target product purified from microbes is provided, and target product sometimes is provided in substantially pure form.

In certain embodiments, a target product (e.g., lycopene for example) is produced with a yield of about 0.30 grams of target product, or greater, per gram of glucose added during a fermentation process (e.g., about 0.31 grams of target product per gram of glucose added, or greater; about 0.32 grams of target product per gram of glucose added, or greater; about 0.33 grams of target product per gram of glucose added, or greater; about 0.34 grams of target product per gram of glucose added, or greater; about 0.35 grams of target product per gram of glucose added, or greater; about 0.36 grams of target product per gram of glucose added, or greater; about 0.37 grams of target product per gram of glucose added, or greater; about 0.38 grams of target product per gram of glucose added, or greater; about 0.39 grams of target product per gram of glucose added, or greater; about 0.40 grams of target product per gram of glucose added, or greater; about 0.41 grams of target product per gram of glucose added, or greater; 0.42 grams of target product per gram of glucose added, or greater; 0.43 grams of target product per gram of glucose added, or greater; 0.44 grams of target product per gram of glucose added, or greater; 0.45 grams of target product per gram of glucose added, or greater; 0.46 grams of target product per gram of glucose added, or greater; 0.47 grams of target product per gram of glucose added, or greater; 0.48 grams of target product per gram of glucose added, or greater; 0.49 grams of target product per gram of glucose added, or greater; 0.50 grams of target product per gram of glucose added, or greater; 0.51 grams of target product per gram of glucose added, or greater; 0.52 grams of target product per gram of glucose added, or greater; 0.53 grams of target product per gram of glucose added, or greater; 0.54 grams of target product per gram of glucose added, or greater; 0.55 grams of target product per gram of glucose added, or greater; 0.56 grams of target product per gram of glucose added, or greater; 0.57 grams of target product per gram of glucose added, or greater; 0.58 grams of target product per gram of glucose added, or greater; 0.59 grams of target product per gram of glucose added, or greater; 0.60 grams of target product per gram of glucose added, or greater; 0.61 grams of target product per gram of glucose added, or greater; 0.62 grams of target product per gram of glucose added, or greater; 0.63 grams of target product per gram of glucose added, or greater; 0.64 grams of target product per gram of glucose added, or greater; 0.65 grams of target product per gram of glucose added, or greater; 0.66 grams of target product per gram of glucose added, or greater; 0.67 grams of target product per gram of glucose added, or greater; 0.68 grams of target product per gram of glucose added, or greater; 0.69 or 0.70 grams of target product per gram of glucose added or greater). In some embodiments, 0.45 grams of target product per gram of glucose added, or greater, is produced during the fermentation process.

### EXAMPLES

The examples set forth below illustrate certain embodiments and do not limit the technology. Certain examples set forth below utilize standard recombinant DNA and other biotechnology protocols known in the art. Many such techniques are described in detail in Maniatis, T., E. F. Fritsch and J. Sambrook (1982) Molecular Cloning: a Laboratory Manual; Cold Spring Harbor Laboratory, Cold Spring Harbor, N.Y. DNA mutagenesis can be accomplished using the Stratagene (San Diego, Calif.) “QuickChange” kit according to the manufacturer's instructions, or by one of the other types of mutagenesis described above.

### Example I

### Preparation of Three Wild Type Lycopene Synthase Gene Constructs

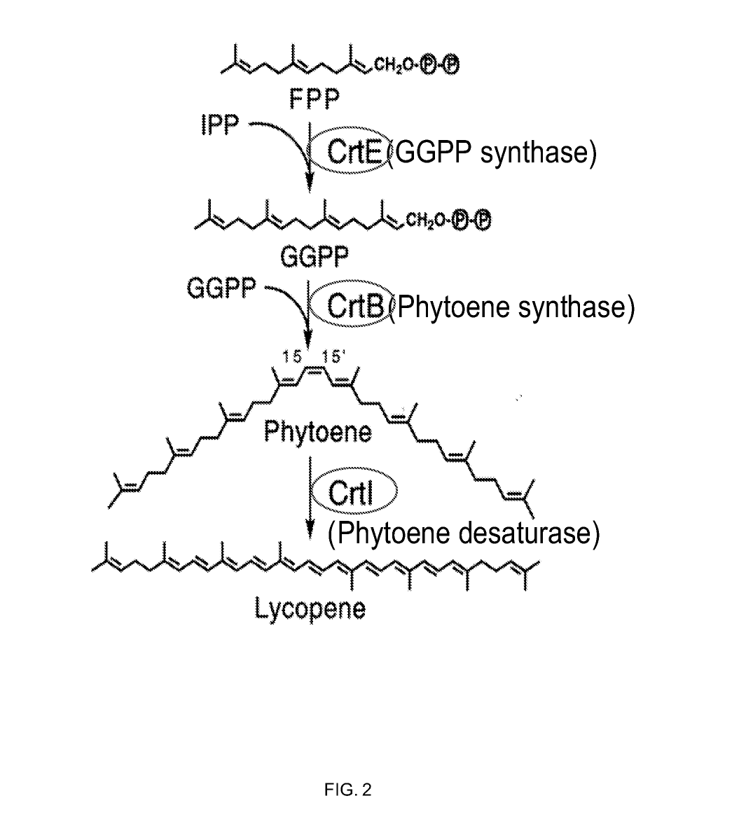

The genes encoding the enzymes geranylgeranyl diphosphate synthase (known as crtE), phytoene synthase (known as crtB), and phytoene desaturase (known as crtI) were isolated from the bacteria Pantoea ananatis, Pantoea agglomerans, and Chronobacter sakazakii. A schematic representation of the intermediates upon which crtE, crtB and crtI act is shown in FIG. 2. Genomic DNA was obtained from the American Type Culture Collection (ATCC) for each bacteria species (ATCC numbers 19321, 33243D, and BAA-894D-5, respectively). Each of the three genes from each of the three species was cloned using standard polymerase chain reaction (PCR) methods. The full length sequence of each gene is publicly available.

Cloning the cDNA of each gene from P. ananatis was accomplished by PCR using the following PCR primers:

crtE:
5′ ACTAGCCGCATATGacggtctgcgcaaaaaaacacg
3′CACCAATTACCGTAGTTGGTTTCATATCTGACCTCCTTTAACTGACGG
CAGCGAGTTTTTTG
crtI:
5′ caaaaaactcgctgccgtcagttaaAGGAGGTCAGATatgaaacc
aactacggtaattggtg
3′GATTGAGTAACGACGGATTATTCATGTAGTCGCTCCTCTCATATCAGA
TCCTCCAGCATCAAAC
crtB:
5′ gtttgatgctggaggatctgatatgaGAGGAGCGACTACatgaataa
tccgtcgttactcaatc
3′ CTTACGGGACTAGTCTAGAGCGGGCGCTGCCAGAGATGC

### Ribosome Binding Sequences:

AGGAGGTCAGAT (RBS1)
GGAGCGACTAC (RBS2)

The crtE 5′ primer contained a NdeI site followed by the 5′ sequence of crtE; the crtE 3′ primer contained the 3′ end of the crtE cDNA sequence, followed by RBS1 sequence and then the 5′ end of the crtI cDNA sequence. The crtI 5′ primer contained the 3′ end of the crtB cDNA sequence followed by RBS1 sequence, and the 5′ end of the crtI sequence. The crtI 3′ primer contained the 3′ end of the crtI cDNA sequence, followed by the RBS2 sequence, and then the 5′ end of the crtB sequence. The crtB 3′ primer contained the 3′ sequence of crtB followed by a SpeI sequence. These primers, when used allowed the creation of three molecules that could later be used as PCR templates to generate a construct containing, 5′ to 3′, a NdeI site, the crtE cDNA, RBS1, the crtI cDNA, RBS2, the crtB cDNA, and a SpeI site.

Each PCR amplification of the individual cDNAs was performed using pfu DNA polymerase (Stratagene Inc., catalog #600672), based on the standard PCR protocol, using a Tm of about 55 degrees Celsius. Each reaction contained the following contents (volumes are approximate):

| | |
|---|---|
| Genomic DNA | 50 nanograms |
| 5′ primer (10 micromolar) | 0.5 microliter |
| 3′ primer (10 micromolar) | 0.5 microliter |
| Pfu DNA polymerase | 0.5 microliter |
| 10 mmol dNTP mix | 1 microliter |
| 10X pfu buffer | 5 microliters |

This mixture was diluted to a final volume of about fifty microliters using distilled water.

The PCR amplification protocol for each reaction was as follows. All temperatures are in degrees Celsius: PCR reaction conditions were: step 1: 95 degrees, 10 minutes; step 2 (30 cycles): 95 degrees, 20 seconds; 55 degrees, 30 seconds; 72 degrees, 30 seconds; step 3: 72 degrees, 5 minutes; step 4: 4 degrees, hold.

All PCR products were column purified using the “DNA Clean & Concentrator-25 Kit” (Zymo Research Inc.) according to the manufacturer's instructions. After purification, each reaction tube was diluted in solution to about 0.1 pmol and all reactions mixtures were then combined.

A full length construct containing the cDNA for each gene in the 5′ to 3′ order crtE, RBS, crtI, RBS, crtB was then prepared using the above cDNAs as templates. The reaction mix for this PCR contained:

| | |
|---|---|
| crtE, crtI and crtB cDNAs | 0.1 pmol of each |
| 5′ crtE primer (10 micromolar) | 0.5 microliter |
| 3′ crtB primer (10 micromolar) | 0.5 microliter |
| Pfu DNA polymerase | 1 microliter |
| 10X Pfu buffer | 5 microliter |
| 10 mmol dNTP mix | 1 microliter |

This mixture was diluted to a final volume of about fifty microliters using distilled water. PCR amplification of the full length construct was performed using the following protocol (all temperatures are in degrees Celsius): PCR reaction conditions were: step 1: 95 degrees, 10 minutes; step 2 (30 cycles): 95 degrees, 20 seconds; 58 degrees, 30 seconds; 72 degrees, 2 minutes; step 3: 72 degrees, 5 minutes; step 4: 4 degrees, hold.

The sequence of the final construct for P. ananatis was:

atgacggtctgcgcaaaaaaacacgttcatctcactcgcgatgctgcggagcagttactggctga
tattgatcgacgccttgatcagttattgcccgtggagggagaacgggatgttgtgggtgccgcga
tgcgtgaaggtgcgctggcaccgggaaaacgtattcgccccatgttgctgttgctgaccgcccgc
gatctgggttgcgctgtcagccatgacggattactggatttggcctgtgcggtggaaatggtcca
cgcggcttcgctgatccttgacgatatgccctgcatggacgatgcgaagctgcggcgcggacgcc
ctaccattcattctcattacggagagcatgtggcaatactggcggcggttgccttgctgagtaaa
gcctttggcgtaattgccgatgcagatggcctcacgccgctggcaaaaaatcgggcggtttctga
actgtcaaacgccatcggcatgcaaggattggttcagggtcagttcaaggatctgtctgaagggg
ataagccgcgcagcgctgaagctattttgatgacgaatcactttaaaaccagcacgctgttttgt
gcctccatgcagatggcctcgattgttgcgaatgcctccagcgaagcgcgtgattgcctgcatcg
tttttcacttgatcttggtcaggcatttcaactgctggacgatttgaccgatggcatgaccgaca
ccggtaaggatagcaatcaggacgccggtaaatcgacgctggtcaatctgttaggcccgagggcg
gttgaagaacgtctgagacaacatcttcagcttgccagtgagcatctctctgcggcctgccaaca
cgggcacgccactcaacattttattcaggcctggtttgacaaaaaactcgctgccgtcagttaaA
GGAGGTCAGATatgaaaccaactacggtaattggtgcaggcttcggtggcctggcactggcaatt
cgtctacaagctgcggggatccccgtcttactgcttgaacaacgtgataaacccggcggtcgggc
ttatgtctacgaggatcaggggtttacctttgatgcaggcccgacggttatcaccgatcccagtg
ccattgaagaactgtttgcactggcaggaaaacagttaaaagagtatgtcgaactgctgccggtt
acgccgttttaccgcctgtgttgggagtcagggaaggtctttaattacgataacgatcaaacccg
gctcgaagcgcagattcagcagtttaatccccgcgatgtcgaaggttatcgtcagtttctggact
attcacgcgcggtgtttaaagaaggctatctaaagctcggtactgtcccttttttatcgttcaga
gacatgcttcgcgccgcacctcaactggcgaaactgcaggcatggagaagcgtttacagtaaggt
tgccagttacatcgaagatgaacatctgcgccaggcgttttctttccactcgctgttggtgggcg
gcaatcccttcgccacctcatccatttatacgttgatacacgcgctggagcgtgagtggggcgtc
tggtttccgcgtggcggcaccggcgcattagttcaggggatgataaagctgtttcaggatctggg
tggcgaagtcgtgttaaacgccagagtcagccatatggaaacgacaggaaacaagattgaagccg
tgcatttagaggacggtcgcaggttcctgacgcaagccgtcgcgtcaaatgcagatgtggttcat
acctatcgcgacctgttaagccagcaccctgccgcggttaagcagtccaacaaactgcagactaa
gcgcatgagtaactctctgtttgtgctctattttggtttgaatcaccatcatgatcagctcgcgc
atcacacggtttgtttcggcccgcgttaccgcgagctgattgacgaaatttttaatcatgatggc
ctcgcagaggacttctcactttatctgcacgcgccctgtgtcacggattcgtcactggcgcctga
aggttgcggcagttactatgtgttggcgccggtgccgcatttaggcaccgcgaacctcgactgga
cggttgaggggccaaaactacgcgaccgtatttttgcgtaccttgagcagcattacatgcctggc
ttacggagtcagctggtcacgcaccggatgtttacgccgtttgattttcgcgaccagcttaatgc
ctatcatggctcagccttttctgtggagcccgttcttacccagagcgcctggtttcggccgcata
accgcgataaaaccattactaatctctacctggtcggcgcaggcacgcatcccggcgcaggcatt
cctggcgtcatcggctcggcaaaagcgacagcaggtttgatgctggaggatctgatatgaGAGGA
GCGACTACatgaataatccgtcgttactcaatcatgcggtcgaaacgatggcagttggctcgaaa
agttttgcgacagcctcaaagttatttgatgcaaaaacccggcgcagcgtactgatgctctacgc
ctggtgccgccattgtgacgatgttattgacgatcagacgctgggctttcaggcccggcagcctg
ccttacaaacgcccgaacaacgtctgatgcaacttgagatgaaaacgcgccaggcctatgcagga
tcgcagatgcacgaaccggcgtttgcggcttttcaggaagtggctatggctcatgatatcgcccc
ggcttacgcgtttgatcatctggaaggcttcgccatggatgtacgcgaagcgcaatacagccaac
tggatgatacgctgcgctattgctatcacgttgcaggcgttgtcggcttgatgatggcgcaaatc
atgggcgtgcgggataacgccacgctggaccgcgcctgtgaccttgggctggcatttcagttgac
caatattgctcgcgatattgtggacgatgcgcatgcgggccgctgttatctgccggcaagctggc
tggagcatgaaggtctgaacaaagagaattatgcggcacctgaaaaccgtcaggcgctgagccgt
atcgcccgtcgtttggtgcaggaagcagaaccttactatttgtctgccacagccggcctggcagg
gttgcccctgcgttccgcctgggcaatcgctacggcgaagcaggtttaccggaaaataggtgtca
aagttgaacaggccggtcagcaagcctgggatcagcggcagtcaacgaccacgcccgaaaaatta
acgctgctgctggccgcctctggtcaggcccttacttcccggatgcgggctcatcctccccgccc
tgcgcatctctggcagcgcccgctctag

For PCR cloning the cDNA from Pantoea agglomerans, the following primers were used:

crtE
5′ ACTAGCCGCATATGatgacggtctgtgcagaacaac
3′
CCAATTACTGTAGTTCTATTCATATCTGACCTCCTTTAACTGACGGCA
GCGAGTTTTTTC
crtI
5′ gaaaaaactcgctgccgtcagttaaAGGAGGTCAGATatgaatagaa
ctacagtaattgg
3′GCTTTTCGATCCCACCTCCATGTAGTCGCTCCTCTCAAGCCAGATCC
TCCAGCATCAATC
crtB
5′ gattgatgctggaggatctggcttgaGAGGAGCGACTACatggaggt
gggatcgaaaagc
3′ CTTACGGGACTAGTTTAAACGGGGCGCTGCCAGAGAT

These primers were designed as described above for P. ananatis in that they permitted the creation of a full length construct containing from 5′ to 3′: a NdeI site, the crtE cDNA, RBS1, the crtI cDNA, RBS2, the crtB cDNA, and a SpeI site. PCR amplification of the individual cDNAs was conducted as set forth above for P. ananatis individual cDNA amplifications. Purification of each cDNA was accomplished as described for the P. ananatis cDNAs, and PCR to prepare the final full length construct was conducted as described above for P. ananatis.

The sequence of the full length construct was:

atgatgacggtctgtgcagaacaacacgtcaatttcatacacagcgatgcagccagcctgttgaa
cgacattgagcaacggcttgatcagcttttaccggttgaaagcgaacgtgacttagtgggcgctg
ccatgcgcgacggtgcgctggcaccaggaaagcgtatccgtccactgctgttgttgctggcagcg
cgcgatctgggctgcaacgccacgcctgccggcctgctcgatctcgcctgcgcggtagagatggt
gcatgccgcatcactgattctggatgacatgccctgcatggatgatgcgcaactgcgtcgcggac
gtccgaccattcattgccagtatggtgaacatgtcgcgattctggccgcggtggccctgctgagt
aaggcattcggcgtggtcgctgcggcagaaggcttaacggcaaccgccagagccgacgctgtggc
agaattatcccacgcagtcggcatgcaggggctggtgcaggggcaatttaaggatctctccgagg
gtgacaagccacgcagcgctgacgccattctgatgaccaatcactataaaaccagcacgctgttc
tgcgcctccatgcagatggcctctatcgtggctgaagcctcaggtgaagcccgcgaacagctgca
ccgtttttcgcttaatcttggtcaggctttccagctactggacgatctcactgacggcatggccg
acaccggtaaagatgcccatcaggatgacgggaaatcaacgctggtgaatctgctgggaccacag
gcggttgaaacgcgactgcgcgatcatctgcgctgcgccagcgagcatctgttatcggcctgcca
ggacggttatgccacacaccattttgttcaggcctggtttgagaaaaaactcgctgccgtcagtt
aaAGGAGGTCAGATatgaatagaactacagtaattggcgcaggctttggtggtctggctctggca
attcgccttcaggcgtcaggcgttcccacccgactgctggagcagcgtgacaagcctggcggccg
ggcttatgtctatcaggatcagggcttcacgtttgatgccggccccacggtaatcaccgatccca
gcgccattgaagagctgttcaccctcgcgggtaaaaagctctctgactatgtcgagctgatgccg
gtgaagccgttttatcgcctctgctgggagtccggcaaggtgttcagttatgacaacgatcagcc
cgcgctggaagcgcagattgccgcgtttaatccgcgtgacgttgaaggatatcgtcgctttctgg
cctattcccgagcggtgtttgctgaaggctatctgaagcttggcaccgtgccgtttctgtcattc
cgcgacatgctgcgggccgcgcctcagctggcaaaacttcaggcgtggcgcagcgtttacagcaa
agtggcgagctacattgaagatgagcatctgcgtcaggccttctctttccactcactgctggtgg
gcggaaatccgtttgccacttcctcaatctataccctgattcatgcgctggaacgtgaatggggc
gtctggttcccgcgcggtggcacgggcgcgctggtgcagggcatggtgaaactgtttgaagatct
gggcggcgaagtggagctcaatgccagcgttgcccggctggagacccaggaaaacagaattaccg
cggtgcacctgaaagatggccgggtcttcccgacccgcgcggttgcctccaacgcagatgtggtt
cacacctaccgcgaactgctgagccagcatcccgcttcgcaggcgcagggacgatcactgcaaaa
caaacgcatgagcaactcactgtttgtgatctattttggcctgaatcatcatcacaatcagctgg
cgcaccacacggtctgctttggtccgcgctatcgtgagttgattgatgagatctttaacaaagat
ggcctggcagaggacttctcgctctatctgcatgcgccctgcgtgaccgatccctcactggcgcc
ggagggctgcggcagctactacgtgctggcgccagttccgcacctcggcaccgccgatatcgact
gggccgttgaaggtccgcgcctgcgcgatcgcatttttgactatctggaacagcactatatgccg
ggcctgcgtagccagttggtcacgcatcgcatcttcacgccgtttgatttccgcgatgagctgaa
tgcgtatcagggttcggccttctcggtggagccgatcctgacgcaaagcgcctggttccggcctc
acaaccgcgataaaaatattaataatctctatctggtcggtgcaggcacgcatcctggcgcgggt
attccaggcgtaattggctcggccaaggctaccgcaggattgatgctggaggatctggcttgaGA
GGAGCGACTACatggaggtgggatcgaaaagctttgccaccgcgtcaaaactgtttggtgccaaa
acccgacgcagcgtgctgatgctctacgcctggtgccgtcactgtgatgatgtgattgacgatca
ggtactgggattcagcaacgatacgccatcgctgcaatccgccgaacagcgcctggcgcagctgg
agatgaaaacgcgtcaggcctatgccggatcccagatgcatgagcccgcctttgcagcctttcag
gaggtggcaatggcacacgatattctgcctgcttacgcttttgatcatctggcgggctttgcgat
ggacgtgcatgagacacgctatcagacgctggatgatacgctgcgttactgttaccacgtcgcgg
gcgtggttggcctgatgatggcgcagattatgggcgtacgcgacaacgccacgctggatcgcgcc
tgcgatctcggtctggcgtttcagctgaccaatattgcgcgcgatatcgttgaagatgctgacgc
gggacgctgctatctgcccgctacgtggctggctgaagaggggcttacccgagagaatctcgccg
atccgcaaaatcgccaggcattaagccgcgtcgcccgccggctggtggaaacggcagagccctat
tatcgatcggcgtcggctggcctgccgggtttaccgctgcgttcagcgtgggcgattgctaccgc
gcagcaggtctaccgtaaaatcggtatgaaggtggttcaggcggcttcacaggcgtgggatcaac
gccagtccaccagcacaccagagaaactggcactgctggtggcggcatcgggtcaggcggttact
tcccgggtggcgcgtcacgctccacgctccgctgatctctggcagcgccccgtttaa

PCR amplification of the crtE, crtI, and crtB cDNAs from Cronobacter sakazakii was accomplished using the procedure set forth above for both P. ananatis and P. agglomerans. The primers used were:

crtE
5′ ACTAGCCGCATAtgaacgctaacgccgtgaaatcttc
3′
GGACCCGATAACAACAGTTTTAGTCATATCTGACCTCCTTCAGCCAAACA
TAGCCAGCTG
crtI
5′ cagctggctatgtttggctgaAGGAGGTCAGATatgactaaaactg
ttgttatcgggtcc
3′
GTCAGCAGCGGTTTGTCACTCATGTAGTCGCTCCTCTCATGCATGCCCCT
CCAGCATCAG
crtB
5′ ctgatgctggaggggcatgcatgaGAGGAGCGACTACatgagtgac
aaaccgctgctgac
3′ CTTACGGGACTAGTCTAAGCTGCAGGCGGTGCTGCGTG

These primers were designed as described above for both Pantoea spp. PCR amplification of the individual cDNAs was accomplished as described above for both Pantoea spp., and PCR amplification to generate the final full length construct incorporating all 3 cDNAs, the RBS sequences and the NdeI and SpeI cloning sites was accomplished as described above.

The sequence of the full length final construct was:

atgaacgctaacgccgtgaaatcttcagggcaggaaatcgaattgcaggcgctgcgcgacgcgct
gcaaacccgccttgacgagcttctgccgccgggccaggagcgcgatctggtctgcgccgcgatgc
gcgaaggcgcgctgacgcccggtaagcgggtgcgcccgctgctgctcattcttgccgcgcgcgat
ctcggctgcgacgccagccagcctgcgttgatggatctcgcctgcgccgtggagatggtgcacgc
cgcgtcgctgatgctcgacgatattccgtgcatggataacgccctgctgcgccgcggcaagccca
ccattcaccgccagtatggcgaaagcgtggcgatcctcgcggcggtggcgctgctgagccgggca
ttcggcgtggtggcgcaggcaaatccgctctccgatagctgcaaaactcaggcggtgagcgagct
ttccagcgccgtcgggttgcaggggctggtgcaggggcagtttcgcgatctcagcgaaggcaacc
aggcccgcagcgccgaggcgatactcgccactaacgatctcaaaaccagcgtgctgtttgacgcc
acgctgcaaatcgccgccatcgccgctggcgcttccgcctcggtgcgccataaacttcgcgagtt
ctcgcgccatctcggccaggcgttccagctgcttgacgatctggcggatggcctgaaccataccg
gtaaagacattaataaagacgccgggaaatcgacgctggtggcgatgcttggcccggaagcggtg
catcagcgcctgcgcgatcacctgctgcgcgccgatgagcatctcaccggtgcctgttcacgcgg
cgcatccacccgccgttttatgtacgcctggtttgataaacagctggctatgtttggctgaAGGA
GGTCAGATatgactaaaactgttgttatcgggtccggctttggcggcctggccctggctatccgc
ttacaggcggcgggcgttcccaccttactgcttgagcagcgcgataaacccggcgggcgggcgta
tgtttatgaagataaagggtttacctttgacgctggcccgacggtgattaccgatccttcggcca
tcgaggagctgttcacgctggccggtaaaaacatcgccgattatgtcgatcttttgcccgtcacg
ccgttctaccgcctgtgctgggagaatggccaggtctttaactacgataacgatcaggcgagcct
tgaggcgcaaatcgcccgcttcaacccgcgcgatgtcgagggctatcgccagttcctggcgtatt
cgcaggcggtgtttaaagaaggctatctgaagcttggcgcggtgccgttcctctcgtttcgcgat
atgttgcgcgcaggcccgcagctcgcgcgtcttcaggcgtggcgcagcgtgtatggcatggtgtc
gaaatttatcgaaaacgatcatctgcgccaggcgttctcgttccattccctgctggtgggcggca
acccgtttgcgacgtcatcaatctatacgcttatccacgcgctggagcgccagtggggcgtctgg
ttcgcccgtggcggcaccggcgcgctggtgcaggggctggtgaagctgtttaccgatctgggcgg
cgagattgaactcaacgccaaagtgacgcgcctcgatacccagggcgacaaaatcagcggcgtga
cgctcgccgacgggcgacgcattcccgcgcgcgccgtggcgtcgaatgcggatgtggtacatacc
tacaacaacttgctgggccatcacccgcgcggcgtctcgcaggcggcctcgctgcgccgcaagcg
gatgagcaactcgctgttcgtgctctatttcgggctcaatcaccaccatagccagctcgcacacc
acacggtctgcttcgggccgcgctacaaagggctgattgaagatatcttcaaacgcgactcgctc
gcggacgacttctcgctctatctgcacgcgccgtgcgtcaccgatccgtcgctggctccgccggg
ctgcggcagctattacgtgctcgccccggtgccgcatctcggcaccgcgaacctgaactgggatg
tcgaagggccgcgcctgcgcgaccggatttttgaatatcttgagcagcactatatgccgggcctt
cgcgatcaactggtgacacaccgtatgttcacgccgttcgatttccgcgaccagctcggcgcgta
tcatggctccgcgttttcggtagagcctatcctcactcagagcgcctggttccgcccgcataacc
gcgacagccgcatcgataacctctatttagtcggcgcgggcacgcaccccggcgcgggcattccg
ggggttatcggctcggcgaaggctaccgccgggctgatgctggaggggcatgcatgaGAGGAGCG
ACTACatgagtgacaaaccgctgctgacgcatgccactgaaaccatcgaggcgggctccaaaagc
tttgccaccgcctcgaaactgtttgacgcgaaaacccgacgcagcgcgctgatgctctacgcctg
gtgtcgccactgcgacgatgtgactgacgggcaggcgcttggttttcgcgcggccgacgcgccga
ctgacaccccgcaggcgcgcatcgccctgctgcgcgcgctgacgcttgaggcttacgcgggcaaa
ccgatgcgcgagccaaatttcgcggcgtttcaggaggtggcgctggcacatcagatcccgcctgc
gctggcgctcgatcatctggaaggtttcgcgatggacgtgcgcgaagaacgctatcacacctttg
atgacacgctgcgctactgttaccacgtggcgggcgtggtggggctgatgatggcgcgcgtgatg
ggcgtgcgcgatgaggccgtgctggatcgcgcctgcgatctgggcctcgcgtttcagctcactaa
cattgcgcgggatatcgttgaagacgccgccatcgggcgctgctatctgcctgaggcgtggcttc
aggaggaagggctttgcgctgacaccttaacagaccgcgcgcaccgcccggcgctggcgcgtctc
gccgcacggctggtggatgaagcggagccgtattacgcctcggcgcgcgccgggcttgccggtct
gccgctacgcagcgcctgggctattgccaccgcgcatggggtctaccgggaaattggcgtaaagg
tgaagcgcgcgggcgttaacgcgtgggaaacgcgtcagggcaccagcaaggccgagaaactggcc
ctgctggcgaaaggcgcggttatggccgtgagttctcgcggcgcgtcgtcgtcgcctcgtccttc
ggcgctctggcagcggccgcgcgcgcaggacgaccgttacgctcacgcagcaccgcctgcagctt
ag

### Example 2

### Preparation of Combinatorial Lycopene Synthase DNA Constructs

A combinatorial library was generated in which constructs containing all combinations of crtE, crtI and crtB cDNAs from each of P. ananatis, P. agglomerans, and C. sakazakii were generated. These constructs contained, from 5′ to 3′, a NdeI cloning site, crtE, RBS1, crtI, RBS2, crtB, and a SpeI cloning site. Each construct was prepared by individually cloning each gene from each organism's genomic DNA using primers designed to generate cDNAs for each gene that would hybridize to each neighboring gene. These primers also contained the sequence encoding a restriction site or a RBS as appropriate.

For the three crtE cDNAs, the 5′ primer contained the Nde1 site at the 5′ end followed by the 5′ sequence of each crtE gene at the 3′ end. The sequence of each of these three primers is set forth in Example 1.

The primers for each of the three species of crtI cDNAs contained, from 5′ to 3′, the final 22-28 bases of a crtE gene followed by the sequence for RBS1, followed by 22-26 bases of the 5′ end of a crtI gene.

The primers for each of the three crtB cDNAs contained, from 5′ to 3′, about 20-26 bases of the 3′ end of a crtI gene, followed by the RBS2 sequence, followed by 22-28 bases of the 5′ end of each crtB cDNA. The 3′ end primers for each construct contained about 20-26 bases of the 3′ sequence of a crtB gene, followed by the sequence of the SpeI restriction site.

The sequence of the primers is set forth below. “Pa” refers to P. ananatis; “Pg” refers to P. agglomerans, “Cs” refers to C. sakazakii, “E” refers to crtE; “I” refers to crtI; “B” refers to crtB. Thus, “PaPgEI” refers to the primer used for creating that portion of a library construct containing the P. ananatis crtE cDNA and the P. agglomerans crtI cDNA.

NdeI-CrtE (P. ananatis)
ACTAGCCGCATATGacggtctgcgcaaaaaaacacg
NdeI-CrtE (P. agglomerans)
ACTAGCCGCATATGatgacggtctgtgcagaacaac
NdeI-CrtE (C. sakzakii)
ACTAGCCGCATAtgaacgctaacgccgtgaaatcttc
PaPgEI
caaaaaactcgctgccgtcagttaaAGGAGGTCAGATatgaatagaact
acagtaattgg
PaPgEI
CCAATTACTGTAGTTCTATTCATATCTGACCTCCTTTAACTGACGGCAG
CGAGTTTTTTG
PaCsEI
caaaaaactcgctgccgtcagttaaAGGAGGTCAGATatgactaaaactg
ttgttatcgg
PaCsEI
CCGATAACAACAGTTTTAGTCATATCTGACCTCCTTTAACTGACGGCAG
CGAGTTTTTTG
PgPaEI
gaaaaaactcgctgccgtcagttaaAGGAGGTCAGATatgaaaccaact
acggtaattgg
PgPaEI
CCAATTACCGTAGTTGGTTTCATATCTGACCTCCTTTAACTGACGGCAGC
GAGTTTTTTC
PgCsEI
gaaaaaactcgctgccgtcagttaaAGGAGGTCAGATatgactaaaactg
ttgttatcgg
PgCsEI
CCGATAACAACAGTTTTAGTCATATCTGACCTCCTTTAACTGACGGCAG
CGAGTTTTTTC
CsPaEI
ataaacagctggctatgtttggctgaAGGAGGTCAGATatgaaaccaac
tacggtaattg
CsPaEI
CAATTACCGTAGTTGGTTTCATATCTGACCTCCTTCAGCCAAACATAGC
CAGCTGTTTAT
CsPgEI
ataaacagctggctatgtttggctgaAGGAGGTCAGATatgaatagaact
acagtaattg
CsPgEI
CAATTACTGTAGTTCTATTCATATCTGACCTCCTTCAGCCAAACATAGCC
AGCTGTTTAT
PaPgIB
gtttgatgctggaggatctgatatgaGAGGAGCGACTACatggaggtggg
atcgaaaag
PaPgIB
CTTTTCGATCCCACCTCCATGTAGTCGCTCCTCTCATATCAGATCCTCC
AGCATCAAAC
PaCsIB
gtttgatgctggaggatctgatatgaGAGGAGCGACTACatgagtgaca
aaccgctgctg
PaCsIB
CAGCAGCGGTTTGTCACTCATGTAGTCGCTCCTCTCATATCAGATCCTCC
AGCATCAAAC
PgPaIB
gattgatgctggaggatctggcttgaGAGGAGCGACTatgaataatccg
tcgttactc
PgPaIB
GAGTAACGACGGATTATTCATAGTCGCTCCTCTCAAGCCAGATCCTCCAG
CATCAATC
PgCsIB
gattgatgctggaggatctggcttgaGAGGAGCGACTACatgagtgaca
aaccgctgctg
PgCsIB
CAGCAGCGGTTTGTCACTCATGTAGTCGCTCCTCTCAAGCCAGATC
CTCCAGCATCAATC
CsPaIB
gatgctggaggggcatgcatgaGAGGAGCGACTACatgaataa
tccgtcgttactcaatc
CsPaIB
GATTGAGTAACGACGGATTATTCATGTAGTCGCTCCTCTCATGCATGCCC
CTCCAGCATC
CsPgIB
gatgctggaggggcatgcatgaGAGGAGCGACTACatggaggtgggatcg
aaaagctttg
CsPgIB
CAAAGCTTTTCGATCCCACCTCCATGTAGTCGCTCCTCTCATGCATGCC
CCTCCAGCATC
SpeI-crtB (P. ananatis)
CTTACGGGACTAGTCTAGAGCGGGCGCTGCCAGAGATGC
SpeI-crtB (P. agglomerans)
CTTACGGGACTAGTTTAAACGGGGCGCTGCCAGAGAT
SpeI-crtB (C. sakazakii)
CTTACGGGACTAGTCTAAGCTGCAGGCGGTGCTGCGTG

Separate PCR amplifications were performed for each of the three genomic DNAs and the corresponding primers.

| | |
|---|---|
| Genomic DNA | 50 ng |
| 5′ primer (10 micromolar) | 0.5 microliter |
| 3′ primer (10 micromolar) | 0.5 microliter |
| Pfu DNA polymerase | 0.5 microliter |
| 10 mmole dNTP mix | 1 microliter |
| 10X pfu buffer | 5 microliter |

PCR reaction conditions were: step 1: 95 degrees, 10 minutes; step 2 (30 cycles): 95 degrees, 20 seconds; 55 degrees, 30 seconds; 72 degrees, 30 seconds; step 3: 72 degrees, 5 minutes; step 4: 4 degrees, hold.

All PCR products were column purified using the “DNA Clean & Concentrator-25 Kit” (Zymo Research Inc.) according to the manufacturer's instructions. The three PCR reaction tubes for each cDNA (crtE, crtI or crtB) to generate three tubes, each tube containing a pool of all PCR products for either crtE cDNA, crtI cDNA or crtB cDNA at a final amount of about 0.1 picomole.

The full length combinatorial library was generated by combining and amplifying all PCR products as follows:

| | |
|---|---|
| crtE pool + crtI pool + crtB pool | 0.1 pmol of each pool |
| 5′ NdeI primer mix (10 micromolar) | 0.5 microliter |
| 3′ SpeI primer mix (10 micromolar) | 0.5 microliter |
| Pfu DNA polymerase | 1 microliter |
| 10X Pfu buffer | 5 microliter |
| 10 mmol dNTP mix | 1 microliter |

The reaction tube was adjusted to a final volume of about 50 microliters using distilled water. PCR reaction conditions were: step 1: 95 degrees, 10 minutes; step 2 (30 cycles): 95 degrees, 20 seconds; 55 degrees, 30 seconds; 72 degrees, 30 seconds; step 3: 72 degrees, 5 minutes; step 4: 4 degrees, hold.

The resulting full-length crtEIB combinatorial library constructs were digested with NdeI and SpeI and column purified as described above. The purified library constructs were then ligated into the vector pCR2.1-TOPO (Life Technologies; Invitrogen, Carlsbad, Calif.) using standard protocols and following the manufacturer's instructions. The ligated Topo-CrtEIB library plasmids were transformed into E. coli DH5 alpha competent cells using methods well known in the art. Single transformants were selected and incubated in LB-kanamycin medium at about 37 degrees Celsius for about 24 hours. Genomic DNA was prepared from each transformant and sequenced using standard methods.

### Example 3

### Quantification of Lycopene Production

Quantitative assays for lycopene production were conducted essentially according to the method set forth in Appl. Microbiol. Biotechnol. (2007) 74: 131-139). About 2 ml of each suspension of transformed E. coli cells were harvested by centrifugation at 14,000 rpm for about 1 minute and washed once with distilled water. The cells were resuspended in about 1 ml of acetone and incubated in the dark at about 55 degrees Celsius for 15 minutes. The samples were then centrifuged at 14,000 rpm for about 10 minutes. The acetone supernatants were transferred to separate tubes, and the lycopene content of each supernatant was quantified by measuring absorbance at 475 nm with a UV spectrophotometer (Jenway 6320D) using lycopene standards (Sigma) as controls. The results of three independent experiments are set forth in Table I below. The nomenclature “lib#” refers to the construct number assayed. The source organisms for each of the 3 crt cDNAs is indicated, as is the average lycopene content (in mg/liter) and standard deviation.

TABLE I

| | crtE | crtI | crtB | Average | STD |
|---|---|---|---|---|---|
| Vector | — | — | — | 0.004 | 0.00321455 |
| P. ananatis | P ana | P ana | P ana | 0.019 | 0.003464102 |
| P. agglomerans | P agg | P agg | P agg | 0.013 | 0.007 |
| C. sakazakii | C sak | C sak | C sak | 0.301 | 0.026501572 |
| lib #15 | P ana | P agg | P ana | 0.008 | 0.004 |
| lib #51 | P ana | C sak | P ana | 0.012 | 0.01 |
| lib #5 | P ana | P ana | P agg | 0.007 | 0.005334918 |
| lib #10 | P ana | P ana | C sak | 0.096 | 0.051870075 |
| lib #2 | P ana | P agg | P agg | 0.009 | 0.000849192 |
| lib #19 | P ana | P agg | C sak | 0.033 | 0.037854198 |
| lib #1 | Pana | C sak | P agg | 0.003 | 0.001381791 |
| lib #14 | P ana | C sak | C sak | 0.012 | 0.005214114 |
| lib #43 | P agg | P agg | P ana | 0.075 | 0.034 |
| lib #6 | P agg | P agg | C sak | 0.087 | 0.025 |
| lib #44 | P agg | P ana | P ana | 0.086 | 0.028429726 |
| lib #40 | P agg | P ana | P agg | 0.006 | 0.006893155 |
| lib #25 | P agg | P ana | C sak | 0.099 | 0.06901325 |
| lib #3 | P agg | C sak | P ana | 0.196 | 0.055799743 |
| lib #24 | P agg | C sak | P agg | 0.013 | 0.003702718 |
| lib #58 | P agg | C sak | C sak | 0.034 | 0.003355338 |
| lib #3-3-1 | C sak | C sak | P ana | 0.419 | 0.085 |
| lib #88 | C sak | P ana | P ana | 0.265 | 0.091 |
| lib #11 | C sak | P ana | P agg | 0.038 | 0.015935491 |
| lib #92 | C sak | P ana | C sak | 0.457 | 0.012335225 |
| lib #7 | C sak | P agg | P ana | 0.295 | 0.101882019 |
| lib #8 | C sak | P agg | P agg | 0.021 | 0.007074688 |
| lib #33 | C sak | P agg | C sak | 0.503 | 0.075338952 |

As can be seen, the constructs labeled “lib#3-3-1”, “lib#92” and “lib#33”, each of which contains lycopene pathway genes from more than one species, produced significantly more lycopene than any of the wild type constructs.

### Example 4

### Examples of Genes with Constitutive Promoters

The following table lists examples of genes that are operably linked to promoters that can be utilized in nucleic acids described herein. The sequences of such promoters are therefore known in the art.

| Gene Symbol/Promoter | |
|---|---|
| designation/ORF designation | Gene function |
| HXT7-390 (The first 390 | High affinity glucose transporter |
| nucleotides of the | |
| HXT7 promoter) | |
| GPD1//YDL022W | NAD-dependent glycerol-3-phosphate |
| | dehydrogenase (also known as DAR1, |
| | HOR1, OSG1 and OSR5) |
| TEF1 | Transcription elongation factor-1 |
| PGK1 | Phosphoglycerate kinase-1 |
| ADH1 | Alcohol dehydrogenase-1 |
| PMA1//YGL008C | Plasma membrane H+-ATPase; also |
| | known as KTI10. |

### Example 5

### Examples of Polynucleotide Regulators

Provided in the tables hereafter are non-limiting examples of regulator polynucleotides that can be utilized in embodiments herein. Such polynucleotides may be utilized in native form or may be modified for use herein. Examples of regulatory polynucleotides include those that are regulated by oxygen levels in a system (e.g., up-regulated or down-regulated by relatively high oxygen levels or relatively low oxygen levels). ORF names in the tables pertain to S. cerevisiae yeast and homologs from other organisms can be tested and utilized. Prokaryotic regulator polynucleotides also can be tested and utilized (e.g., ArcA and FNR from E. coli).

Regulated Yeast Promoters - Up-regulated by oxygen

| | | Relative | Relative | |
|---|---|---|---|---|
| | | mRNA | mRNA | |
| ORF | Gene | level | level | |
| name | name | (Aerobic) | (Anaerobic) | Ratio |
| YPL275W | | 4389 | 30 | 219.5 |
| YPL276W | | 2368 | 30 | 118.4 |
| YDR256C | CTA1 | 2076 | 30 | 103.8 |
| YHR096C | HXT5 | 1846 | 30 | 72.4 |
| YDL218W | | 1189 | 30 | 59.4 |
| YCR010C | | 1489 | 30 | 48.8 |
| YOR161C | | 599 | 30 | 29.9 |
| YPL200W | | 589 | 30 | 29.5 |
| YGR110W | | 1497 | 30 | 27 |
| YNL237W | YTP1 | 505 | 30 | 25.2 |
| YBR116C | | 458 | 30 | 22.9 |
| YOR348C | PUT4 | 451 | 30 | 22.6 |
| YBR117C | TKL2 | 418 | 30 | 20.9 |
| YLL052C | | 635 | 30 | 20 |
| YNL195C | | 1578 | 30 | 19.4 |
| YPR193C | | 697 | 30 | 15.7 |
| YDL222C | | 301 | 30 | 15 |
| YNL335W | | 294 | 30 | 14.6 |
| YPL036W | PMA2 | 487 | 30 | 12.8 |
| YML122C | | 206 | 30 | 10.3 |
| YGR067C | | 236 | 30 | 10.2 |
| YPR192W | | 204 | 30 | 10.2 |
| YNL014W | | 828 | 30 | 9.8 |
| YFL061W | | 256 | 30 | 9.1 |
| YNR056C | | 163 | 30 | 8.1 |
| YOR186W | | 153 | 30 | 7.6 |
| YDR222W | | 196 | 30 | 6.5 |
| YOR338W | | 240 | 30 | 6.3 |
| YPR200C | | 113 | 30 | 5.7 |
| YMR018W | | 778 | 30 | 5.2 |
| YOR364W | | 123 | 30 | 5.1 |
| YNL234W | | 93 | 30 | 4.7 |
| YNR064C | | 85 | 30 | 4.2 |
| YGR213C | RTA1 | 104 | 30 | 4 |
| YCL064C | CHA1 | 80 | 30 | 4 |
| YOL154W | | 302 | 30 | 3.9 |
| YPR150W | | 79 | 30 | 3.9 |
| YPR196W | MAL63 | 30 | 30 | 3.6 |
| YDR420W | HKR1 | 221 | 30 | 3.5 |
| YJL216C | | 115 | 30 | 3.5 |
| YNL270C | ALP1 | 67 | 30 | 3.3 |
| YHL016C | DUR3 | 224 | 30 | 3.2 |
| YOL131W | | 230 | 30 | 3 |
| YOR077W | RTS2 | 210 | 30 | 3 |
| YDR536W | STL1 | 55 | 30 | 2.7 |
| YNL150W | | 78 | 30 | 2.6 |
| YHR212C | | 149 | 30 | 2.4 |
| YJL108C | | 106 | 30 | 2.4 |
| YGR069W | | 49 | 30 | 2.4 |
| YDR106W | | 60 | 30 | 2.3 |
| YNR034W | SOL1 | 197 | 30 | 2.2 |
| YEL073C | | 104 | 30 | 2.1 |
| YOL141W | | 81 | 30 | 1.8 |

Regulated Yeast Promoters - Down-regulated by oxygen

| | | Relative | Relative | |
|---|---|---|---|---|
| | Gene | mRNA level | mRNA level | |
| ORF name | name | (Aerobic) | (Anaerobic) | Ratio |
| YJR047C | ANB1 | 30 | 4901 | 231.1 |
| YMR319C | FET4 | 30 | 1159 | 58 |
| YPR194C | | 30 | 982 | 49.1 |
| YIR019C | STA1 | 30 | 981 | 22.8 |
| YHL042W | | 30 | 608 | 12 |
| YHR210C | | 30 | 552 | 27.6 |
| YHR079B | SAE3 | 30 | 401 | 2.7 |
| YGL162W | STO1 | 30 | 371 | 9.6 |
| YHL044W | | 30 | 334 | 16.7 |
| YOL015W | | 30 | 320 | 6.1 |
| YCLX07W | | 30 | 292 | 4.2 |
| YIL013C | PDR11 | 30 | 266 | 10.6 |
| YDR046C | | 30 | 263 | 13.2 |
| YBR040W | FIG1 | 30 | 257 | 12.8 |
| YLR040C | | 30 | 234 | 2.9 |
| YOR255W | | 30 | 231 | 11.6 |
| YOL014W | | 30 | 229 | 11.4 |
| YAR028W | | 30 | 212 | 7.5 |
| YER089C | | 30 | 201 | 6.2 |
| YFL012W | | 30 | 193 | 9.7 |
| YDR539W | | 30 | 187 | 3.4 |
| YHL043W | | 30 | 179 | 8.9 |
| YJR162C | | 30 | 173 | 6 |
| YMR165C | SMP2 | 30 | 147 | 3.5 |
| YER106W | | 30 | 145 | 7.3 |
| YDR541C | | 30 | 140 | 7 |
| YCRX07W | | 30 | 138 | 3.3 |
| YHR048W | | 30 | 137 | 6.9 |
| YCL021W | | 30 | 136 | 6.8 |
| YOL160W | | 30 | 136 | 6.8 |
| YCRX08W | | 30 | 132 | 6.6 |
| YMR057C | | 30 | 109 | 5.5 |
| YDR540C | | 30 | 83 | 4.2 |
| YOR378W | | 30 | 78 | 3.9 |
| YBR085W | AAC3 | 45 | 1281 | 28.3 |
| YER188W | | 47 | 746 | 15.8 |
| YLL065W | GIN11 | 50 | 175 | 3.5 |
| YDL241W | | 58 | 645 | 11.1 |
| YBR238C | | 59 | 274 | 4.6 |
| YCR048W | ARE1 | 60 | 527 | 8.7 |
| YOL165C | | 60 | 306 | 5.1 |
| YNR075W | | 60 | 251 | 4.2 |
| YJL213W | | 60 | 250 | 4.2 |
| YPL265W | DIP5 | 61 | 772 | 12.7 |
| YDL093W | PMT5 | 62 | 353 | 5.7 |
| YKR034W | DAL80 | 63 | 345 | 5.4 |
| YKR053C | | 66 | 1268 | 19.3 |
| YJR147W | | 68 | 281 | 4.1 |

Known and putative DNA binding motifs

| Regulator | Known Consensus Motif |
|---|---|
| Abf1 | TCRNNNNNNACG |
| Cbf1 | RTCACRTG |
| Gal4 | CGGNNNNNNNNNNNCCG |
| Gcn4 | TGACTCA |
| Gcr1 | CTTCC |
| Hap2 | CCAATNA |
| Hap3 | CCAATNA |
| Hap4 | CCAATNA |
| Hsf1 | GAANNTTCNNGAA |
| Ino2 | ATGTGAAA |
| Mata(A1) | TGATGTANNT |
| Mcm1 | CCNNNWWRGG |
| Mig1 | WWWWSYGGGG |
| Pho4 | CACGTG |
| Rap1 | RMACCCANNCAYY |
| Reb1 | CGGGTRR |
| Ste12 | TGAAACA |
| Swi4 | CACGAAA |
| Swi6 | CACGAAA |
| Yap1 | TTACTAA |

Putative DNA Binding Motifs

| | Best Motif (scored | Best Motif (scored by |
|---|---|---|
| Regulator | by E-value) | Hypergeometric) |
| Abf1 | TYCGT--R-ARTGAYA | TYCGT--R-ARTGAYA |
| Ace2 | RRRAARARAA-A-RARAA | GTGTGTGTGTGTGTG |
| Adr1 | A-AG-GAGAGAG-GGCAG | YTSTYSTT-TTGYTWTT |
| Arg80 | T--CCW-TTTKTTTC | GCATGACCATCCACG |
| Arg81 | AAAAARARAAAARMA | GSGAYARMGGAMAAAAA |
| Aro80 | YKYTYTTYTT----KY | TRCCGAGRYW-SSSGCGS |
| Ash1 | CGTCCGGCGC | CGTCCGGCGC |
| Azf1 | GAAAAAGMAAAAAAA | AARWTSGARG-A--CSAA |
| Bas1 | TTTTYYTTYTTKY-TY-T | CS-CCAATGK--CS |
| Cad1 | CATKYTTTTTTKYTY | GCT-ACTAAT |
| Cbf1 | CACGTGACYA | CACGTGACYA |
| Cha4 | CA---ACACASA-A | CAYAMRTGY-C |
| Cin5 | none | none |
| Crz1 | GG-A-A--AR-ARGGC- | TSGYGRGASA |
| Cup9 | TTTKYTKTTY-YTTTKTY | K-C-C---SCGCTACKGC |
| Dal81 | WTTKTTTTTYTTTTT-T | SR-GGCMCGGC-SSG |
| Dal82 | TTKTTTTYTTC | TACYACA-CACAWGA |
| Dig1 | AAA--RAA-GARRAA-AR | CCYTG-AYTTCW-CTTC |
| Dot6 | GTGMAK-MGRA-G-G | GTGMAK-MGRA-G-G |
| Fhl1 | -TTWACAYCCRTACAY-Y | -TTWACAYCCRTACAY-Y |
| Fkh1 | TTT-CTTTKYTT-YTTTT | AAW-RTAAAYARG |
| Fkh2 | AAARA-RAAA-AAAR-AA | GG-AAWA-GTAAACAA |
| Fzf1 | CACACACACACACACAC | SASTKCWCTCKTCGT |
| Gal4 | TTGCTTGAACGSATGCCA | TTGCTTGAACGSATGCCA |
| Gal4 (Gal) | YCTTTTTTTTYTTYYKG | CGGM---CW-Y--CCCG |
| Gat1 | none | none |
| Gat3 | RRSCCGMCGMGRCGCGCS | RGARGTSACGCAKRTTCT |
| Gcn4 | AAA-ARAR-RAAAARRAR | TGAGTCAY |
| Gcr1 | GGAAGCTGAAACGYMWRR | GGAAGCTGAAACGYMWRR |
| Gcr2 | GGAGAGGCATGATGGGGG | AGGTGATGGAGTGCTCAG |
| Gln3 | CT-CCTTTCT | GKCTRR-RGGAGA-GM |
| Grf10 | GAAARRAAAAAAMRMARA | -GGGSG-T-SYGT-CGA |
| Gts1 | G-GCCRS--TM | AG-AWGTTTTTGWCAAMA |
| Haa1 | none | none |
| Hal9 | TTTTTTYTTTTY-KTTTT | KCKSGCAGGCWTTKYTCT |
| Hap2 | YTTCTTTTYT-Y-C-KT- | G-CCSART-GC |
| Hap3 | T-SYKCTTTTCYTTY | SGCGMGGG--CC-GACCG |
| Hap4 | STT-YTTTY-TTYTYYYY | YCT-ATTSG-C-GS |
| Hap5 | YK-TTTWYYTC | T-TTSMTT-YTTTCCK-C |
| Hir1 | AAAA-A-AARAR-AG | CCACKTKSGSCCT-S |
| Hir2 | WAAAAAAGAAAA-AAAAR | CRSGCYWGKGC |
| Hms1 | AAA-GG-ARAM | -AARAAGC-GGGCAC-C |
| Hsf1 | TYTTCYAGAA--TTCY | TYTTCYAGAA--TTCY |
| Ime4 | CACACACACACACACACA | CACACACACACACACACA |
| Ino2 | TTTYCACATGC | SCKKCGCKSTSSTTYAA |
| Ino4 | G--GCATGTGAAAA | G--GCATGTGAAAA |
| Ixr1 | GAAAA-AAAAAAAARA-A | CTTTTTTTYYTSGCC |
| Leu3 | GAAAAARAARAA-AA | GCCGGTMMCGSYC-- |
| Mac1 | YTTKT--TTTTTYTYTTT | A--TTTTTYTTKYGC |
| Mal13 | GCAG-GCAGG | AAAC-TTTATA-ATACA |
| Mal33 | none | none |
| Mata1 | GCCC-C | CAAT-TCT-CK |
| Mbp1 | TTTYTYKTTT-YYTTTTT | G-RR-A-ACGCGT-R |
| Mcm1 | TTTCC-AAW-RGGAAA | TTTCC-AAW-RGGAAA |
| Met31 | YTTYYTTYTTTTYTYTTC | |
| Met4 | MTTTTTYTYTYTTC | |
| Mig1 | TATACA-AGMKRTATATG | |
| Mot3 | TMTTT-TY-CTT-TTTWK | |
| Msn1 | KT--TTWTTATTCC-C | |
| Msn2 | ACCACC | |
| Msn4 | R--AAAA-RA-AARAAAT | |
| Mss11 | TTTTTTTTCWCTTTKYC | |
| Ndd1 | TTTY-YTKTTTY-YTTYT | |
| Nrg1 | TTY--TTYTT-YTTTYYY | |
| Pdr1 | T-YGTGKRYGT-YG | |
| Phd1 | TTYYYTTTTTYTTTTYTT | |
| Pho4 | GAMAAAAAARAAAAR | |
| Put3 | CYCGGGAAGCSAMM-CCG | |
| Rap1 | GRTGYAYGGRTGY | |
| Rcs1 | KMAARAAAAARAAR | |
| Reb1 | RTTACCCGS | |
| Rfx1 | AYGRAAAARARAAAARAA | |
| Rgm1 | GGAKSCC-TTTY-GMRTA | |
| Rgt1 | CCCTCC | |
| Rim101 | GCGCCGC | |
| Rlm1 | TTTTC-KTTTYTTTTTC | |
| Rme1 | ARAAGMAGAAARRAA | |
| Rox1 | YTTTTCTTTTY-TTTTT | |
| Rph1 | ARRARAAAGG- | |
| Rtg1 | YST-YK-TYTT-CTCCCM | |
| Rtg3 | GARA-AAAAR-RAARAAA | |
| Sfl1 | CY--GGSSA-C | |
| Sfp1 | CACACACACACACAYA | |
| Sip4 | CTTYTWTTKTTKTSA | |
| Skn7 | YTTYYYTYTTTYTYYTTT | |
| Sko1 | none | |
| Smp1 | AMAAAAARAARWARA-AA | |
| Sok2 | ARAAAARRAAAAAG-RAA | |
| Stb1 | RAARAAAAARCMRSRAAA | |
| Ste12 | TTYTKTYTY-TYYKTTTY | |
| Stp1 | GAAAAMAA-AAAAA-AAA | |
| Stp2 | YAA-ARAARAAAAA-AAM | |
| Sum1 | TY-TTTTTTYTTTTT-TK | |
| Swi4 | RAARAARAAA-AA-R-AA | |
| Swi5 | CACACACACACACACACA | |
| Swi6 | RAARRRAAAAA-AAAMAA | |
| Thi2 | GCCAGACCTAC | |
| Uga3 | GG-GGCT | |
| Yap1 | TTYTTYTTYTTTY-YTYT | |
| Yap3 | none | |
| Yap5 | YKSGCGCGYCKCGKCGGS | |
| Yap6 | TTTTYYTTTTYYYYKTT | |
| Yap7 | none | |
| Yfl044c | TTCTTKTYYTTTT | |
| Yjl206c | TTYTTTTYTYYTTTYTTT | |
| Zap1 | TTGCTTGAACGGATGCCA | |
| Zms1 | MG-MCAAAAATAAAAS | |

Transcriptional repressors

| Associated | |
|---|---|
| Gene(s) | Description(s) |
| WHI5 | Repressor of G1 transcription that binds to SCB binding factor (SBF) |
| | at SCB target promoters in early G1; phosphorylation of Whi5p by |
| | the CDK, Cln3p/Cdc28p relieves repression and promoter binding by |
| | Whi5; periodically expressed in G1 |
| TUP1 | General repressor of transcription, forms complex with Cyc8p, |
| | involved in the establishment of repressive chromatin structure |
| | through interactions with histones H3 and H4, appears to enhance |
| | expression of some genes |
| ROX1 | Heme-dependent repressor of hypoxic genes; contains an HMG |
| | domain that is responsible for DNA bending activity |
| SFL1 | Transcriptional repressor and activator; involved in repression of |
| | flocculation-related genes, and activation of stress responsive |
| | genes; negatively regulated by cAMP-dependent protein kinase A |
| | subunit Tpk2p |
| RIM101 | Transcriptional repressor involved in response to pH and in cell wall |
| | construction; required for alkaline pH-stimulated haploid invasive |
| | growth and sporulation; activated by proteolytic processing; similar to A. |
| | nidulans PacC |
| RDR1 | Transcriptional repressor involved in the control of multidrug |
| | resistance; negatively regulates expression of the PDR5 gene; |
| | member of the Gal4p family of zinc cluster proteins |
| SUM1 | Transcriptional repressor required for mitotic repression of middle |
| | sporulation-specific genes; also acts as general replication initiation |
| | factor; involved in telomere maintenance, chromatin silencing; |
| | regulated by pachytene checkpoint |
| XBP1 | Transcriptional repressor that binds to promoter sequences of the |
| | cyclin genes, CYS3, and SMF2; expression is induced by stress or |
| | starvation during mitosis, and late in meiosis; member of the |
| | Swi4p/Mbp1p family; potential Cdc28p substrate |
| NRG2 | Transcriptional repressor that mediates glucose repression and |
| | negatively regulates filamentous growth; has similarity to Nrg1p |
| NRG1 | Transcriptional repressor that recruits the Cyc8p-Tup1p complex to |
| | promoters; mediates glucose repression and negatively regulates a |
| | variety of processes including filamentous growth and alkaline pH |
| | response |
| CUP9 | Homeodomain-containing transcriptional repressor of PTR2, which |
| | encodes a major peptide transporter; imported peptides activate |
| | ubiquitin-dependent proteolysis, resulting in degradation of Cup9p |
| | and de-repression of PTR2 transcription |
| YOX1 | Homeodomain-containing transcriptional repressor, binds to Mcm1p |
| | and to early cell cycle boxes (ECBs) in the promoters of cell cycle- |
| | regulated genes expressed in M/G1 phase; expression is cell cycle- |
| | regulated; potential Cdc28p substrate |
| RFX1 | Major transcriptional repressor of DNA-damage-regulated genes, |
| | recruits repressors Tup1p and Cyc8p to their promoters; involved in |
| | DNA damage and replication checkpoint pathway; similar to a family |
| | of mammalian DNA binding RFX1-4 proteins |
| MIG3 | Probable transcriptional repressor involved in response to toxic |
| | agents such as hydroxyurea that inhibit ribonucleotide reductase; |
| | phosphorylation by Snf1p or the Mec1p pathway inactivates Mig3p, |
| | allowing induction of damage response genes |
| RGM1 | Putative transcriptional repressor with proline-rich zinc fingers; |
| | overproduction impairs cell growth |
| YHP1 | One of two homeobox transcriptional repressors (see also Yox1p), |
| | that bind to Mcm1p and to early cell cycle box (ECB) elements of |
| | cell cycle regulated genes, thereby restricting ECB-mediated |
| | transcription to the M/G1 interval |
| HOS4 | Subunit of the Set3 complex, which is a meiotic-specific repressor of |
| | sporulation specific genes that contains deacetylase activity; |
| | potential Cdc28p substrate |
| CAF20 | Phosphoprotein of the mRNA cap-binding complex involved in |
| | translational control, repressor of cap-dependent translation |
| | initiation, competes with eIF4G for binding to eIF4E |
| SAP1 | Putative ATPase of the AAA family, interacts with the Sin1p |
| | transcriptional repressor in the two-hybrid system |
| SET3 | Defining member of the SET3 histone deacetylase complex which is |
| | a meiosis-specific repressor of sporulation genes; necessary for |
| | efficient transcription by RNAPII; one of two yeast proteins that |
| | contains both SET and PHD domains |
| RPH1 | JmjC domain-containing histone demethylase which can specifically |
| | demethylate H3K36 tri- and dimethyl modification states; |
| | transcriptional repressor of PHR1; Rph1p phosphorylation during |
| | DNA damage is under control of the MEC1-RAD53 pathway |
| YMR181C | Protein of unknown function; mRNA transcribed as part of a |
| | bicistronic transcript with a predicted transcriptional repressor |
| | RGM1/YMR182C; mRNA is destroyed by nonsense-mediated decay |
| | (NMD); YMR181C is not an essential gene |
| YLR345W | Similar to 6-phosphofructo-2-kinase/fructose-2,6-bisphosphatase |
| | enzymes responsible for the metabolism of fructoso-2,6- |
| | bisphosphate; mRNA expression is repressed by the Rfx1p-Tup1p- |
| | Ssn6p repressor complex; YLR345W is not an essential gene |
| MCM1 | Transcription factor involved in cell-type-specific transcription and |
| | pheromone response; plays a central role in the formation of both |
| | repressor and activator complexes |
| PHR1 | DNA photolyase involved in photoreactivation, repairs pyrimidine |
| | dimers in the presence of visible light; induced by DNA damage; |
| | regulated by transcriptional repressor Rph1p |
| HOS2 | Histone deacetylase required for gene activation via specific |
| | deacetylation of lysines in H3 and H4 histone tails; subunit of the |
| | Set3 complex, a meiotic-specific repressor of sporulation specific |
| | genes that contains deacetylase activity |
| RGT1 | Glucose-responsive transcription factor that regulates expression of |
| | several glucose transporter (HXT) genes in response to glucose; |
| | binds to promoters and acts both as a transcriptional activator and |
| | repressor |
| SRB7 | Subunit of the RNA polymerase II mediator complex; associates with |
| | core polymerase subunits to form the RNA polymerase II |
| | holoenzyme; essential for transcriptional regulation; target of the |
| | global repressor Tup1p |
| GAL11 | Subunit of the RNA polymerase II mediator complex; associates with |
| | core polymerase subunits to form the RNA polymerase II |
| | holoenzyme; affects transcription by acting as target of activators |
| | and repressors |

Transcriptional activators

| Associated | |
|---|---|
| Gene(s) | Description(s) |
| SKT5 | Activator of Chs3p (chitin synthase III), recruits Chs3p to the bud neck |
| | via interaction with Bni4p; has similarity to Shc1p, which activates |
| | Chs3p during sporulation |
| MSA1 | Activator of G1-specific transcription factors, MBF and SBF, that |
| | regulates both the timing of G1-specific gene transcription, and cell |
| | cycle initiation; potential Cdc28p substrate |
| AMA1 | Activator of meiotic anaphase promoting complex (APC/C); Cdc20p |
| | family member; required for initiation of spore wall assembly; required |
| | for Clb1p degradation during meiosis |
| STB5 | Activator of multidrug resistance genes, forms a heterodimer with |
| | Pdr1p; contains a Zn(II)2Cys6 zinc finger domain that interacts with a |
| | PDRE (pleotropic drug resistance element) in vitro; binds Sin3p in a |
| | two-hybrid assay |
| RRD2 | Activator of the phosphotyrosyl phosphatase activity of PP2A, peptidyl- |
| | prolyl cis/trans-isomerase; regulates G1 phase progression, the |
| | osmoresponse, microtubule dynamics; subunit of the Tap42p-Pph21p- |
| | Rrd2p complex |
| BLM10 | Proteasome activator subunit; found in association with core particles, |
| | with and without the 19S regulatory particle; required for resistance to |
| | bleomycin, may be involved in protecting against oxidative damage; |
| | similar to mammalian PA200 |
| SHC1 | Sporulation-specific activator of Chs3p (chitin synthase III), required for |
| | the synthesis of the chitosan layer of ascospores; has similarity to |
| | Skt5p, which activates Chs3p during vegetative growth; |
| | transcriptionally induced at alkaline pH |
| NDD1 | Transcriptional activator essential for nuclear division; localized to the |
| | nucleus; essential component of the mechanism that activates the |
| | expression of a set of late-S-phase-specific genes |
| IMP2′ | Transcriptional activator involved in maintenance of ion homeostasis |
| | and protection against DNA damage caused by bleomycin and other |
| | oxidants, contains a C-terminal leucine-rich repeat |
| LYS14 | Transcriptional activator involved in regulation of genes of the lysine |
| | biosynthesis pathway; requires 2-aminoadipate semialdehyde as co- |
| | inducer |
| MSN1 | Transcriptional activator involved in regulation of invertase and |
| | glucoamylase expression, invasive growth and pseudohyphal |
| | differentiation, iron uptake, chromium accumulation, and response to |
| | osmotic stress; localizes to the nucleus |
| HAA1 | Transcriptional activator involved in the transcription of TPO2, YRO2, |
| | and other genes putatively encoding membrane stress proteins; |
| | involved in adaptation to weak acid stress |
| UGA3 | Transcriptional activator necessary for gamma-aminobutyrate (GABA)- |
| | dependent induction of GABA genes (such as UGA1, UGA2, UGA4); |
| | zinc-finger transcription factor of the Zn(2)-Cys(6) binuclear cluster |
| | domain type; localized to the nucleus |
| GCR1 | Transcriptional activator of genes involved in glycolysis; DNA-binding |
| | protein that interacts and functions with the transcriptional activator |
| | Gcr2p |
| GCR2 | Transcriptional activator of genes involved in glycolysis; interacts and |
| | functions with the DNA-binding protein Gcr1p |
| GAT1 | Transcriptional activator of genes involved in nitrogen catabolite |
| | repression; contains a GATA-1-type zinc finger DNA-binding motif; |
| | activity and localization regulated by nitrogen limitation and Ure2p |
| GLN3 | Transcriptional activator of genes regulated by nitrogen catabolite |
| | repression (NCR), localization and activity regulated by quality of |
| | nitrogen source |
| PUT3 | Transcriptional activator of proline utilization genes, constitutively binds |
| | PUT1 and PUT2 promoter sequences and undergoes a conformational |
| | change to form the active state; has a Zn(2)-Cys(6) binuclear cluster |
| | domain |
| ARR1 | Transcriptional activator of the basic leucine zipper (bZIP) family, |
| | required for transcription of genes involved in resistance to arsenic |
| | compounds |
| PDR3 | Transcriptional activator of the pleiotropic drug resistance network, |
| | regulates expression of ATP-binding cassette (ABC) transporters |
| | through binding to cis-acting sites known as PDREs (PDR responsive |
| | elements) |
| MSN4 | Transcriptional activator related to Msn2p; activated in stress |
| | conditions, which results in translocation from the cytoplasm to the |
| | nucleus; binds DNA at stress response elements of responsive genes, |
| | inducing gene expression |
| MSN2 | Transcriptional activator related to Msn4p; activated in stress |
| | conditions, which results in translocation from the cytoplasm to the |
| | nucleus; binds DNA at stress response elements of responsive genes, |
| | inducing gene expression |
| PHD1 | Transcriptional activator that enhances pseudohyphal growth; |
| | regulates expression of FLO11, an adhesin required for pseudohyphal |
| | filament formation; similar to StuA, an A. nidulans developmental |
| | regulator; potential Cdc28p substrate |
| FHL1 | Transcriptional activator with similarity to DNA-binding domain of |
| | Drosophila forkhead but unable to bind DNA in vitro; required for rRNA |
| | processing; isolated as a suppressor of splicing factor prp4 |
| VHR1 | Transcriptional activator, required for the vitamin H-responsive element |
| | (VHRE) mediated induction of VHT1 (Vitamin H transporter) and BIO5 |
| | (biotin biosynthesis intermediate transporter) in response to low biotin |
| | concentrations |
| CDC20 | Cell-cycle regulated activator of anaphase-promoting |
| | complex/cyclosome (APC/C), which is required for |
| | metaphase/anaphase transition; directs ubiquitination of mitotic cyclins, |
| | Pds1p, and other anaphase inhibitors; potential Cdc28p substrate |
| CDH1 | Cell-cycle regulated activator of the anaphase-promoting |
| | complex/cyclosome (APC/C), which directs ubiquitination of cyclins |
| | resulting in mitotic exit; targets the APC/C to specific substrates |
| | including Cdc20p, Ase1p, Cin8p and Fin1p |
| AFT2 | Iron-regulated transcriptional activator; activates genes involved in |
| | intracellular iron use and required for iron homeostasis and resistance |
| | to oxidative stress; similar to Aft1p |
| MET4 | Leucine-zipper transcriptional activator, responsible for the regulation |
| | of the sulfur amino acid pathway, requires different combinations of the |
| | auxiliary factors Cbf1p, Met28p, Met31p and Met32p |
| CBS2 | Mitochondrial translational activator of the COB mRNA; interacts with |
| | translating ribosomes, acts on the COB mRNA 5′-untranslated leader |
| CBS1 | Mitochondrial translational activator of the COB mRNA; membrane |
| | protein that interacts with translating ribosomes, acts on the COB |
| | mRNA 5′-untranslated leader |
| CBP6 | Mitochondrial translational activator of the COB mRNA; |
| | phosphorylated |
| PET111 | Mitochondrial translational activator specific for the COX2 mRNA; |
| | located in the mitochondrial inner membrane |
| PET494 | Mitochondrial translational activator specific for the COX3 mRNA, acts |
| | together with Pet54p and Pet122p; located in the mitochondrial inner |
| | membrane |
| PET122 | Mitochondrial translational activator specific for the COX3 mRNA, acts |
| | together with Pet54p and Pet494p; located in the mitochondrial inner |
| | membrane |
| RRD1 | Peptidyl-prolyl cis/trans-isomerase, activator of the phosphotyrosyl |
| | phosphatase activity of PP2A; involved in G1 phase progression, |
| | microtubule dynamics, bud morphogenesis and DNA repair; subunit of |
| | the Tap42p-Sit4p-Rrd1p complex |
| YPR196W | Putative maltose activator |
| POG1 | Putative transcriptional activator that promotes recovery from |
| | pheromone induced arrest; inhibits both alpha-factor induced G1 arrest |
| | and repression of CLN1 and CLN2 via SCB/MCB promoter elements; |
| | potential Cdc28p substrate; SBF regulated |
| MSA2 | Putative transcriptional activator, that interacts with G1-specific |
| | transcription factor, MBF and G1-specific promoters; ortholog of |
| | Msa2p, an MBF and SBF activator that regulates G1-specific |
| | transcription and cell cycle initiation |
| PET309 | Specific translational activator for the COX1 mRNA, also influences |
| | stability of intron-containing COX1 primary transcripts; localizes to the |
| | mitochondrial inner membrane; contains seven pentatricopeptide |
| | repeats (PPRs) |
| TEA1 | Ty1 enhancer activator required for full levels of Ty enhancer-mediated |
| | transcription; C6 zinc cluster DNA-binding protein |
| PIP2 | Autoregulatory oleate-specific transcriptional activator of peroxisome |
| | proliferation, contains Zn(2)-Cys(6) cluster domain, forms heterodimer |
| | with Oaf1p, binds oleate response elements (OREs), activates beta- |
| | oxidation genes |
| CHA4 | DNA binding transcriptional activator, mediates serine/threonine |
| | activation of the catabolic L-serine (L-threonine) deaminase (CHA1); |
| | Zinc-finger protein with Zn[2]-Cys[6] fungal-type binuclear cluster |
| | domain |
| SFL1 | Transcriptional repressor and activator; involved in repression of |
| | flocculation-related genes, and activation of stress responsive genes; |
| | negatively regulated by cAMP-dependent protein kinase A subunit |
| | Tpk2p |
| RDS2 | Zinc cluster transcriptional activator involved in conferring resistance to |
| | ketoconazole |
| CAT8 | Zinc cluster transcriptional activator necessary for derepression of a |
| | variety of genes under non-fermentative growth conditions, active after |
| | diauxic shift, binds carbon source responsive elements |
| ARO80 | Zinc finger transcriptional activator of the Zn2Cys6 family; activates |
| | transcription of aromatic amino acid catabolic genes in the presence of |
| | aromatic amino acids |
| SIP4 | C6 zinc cluster transcriptional activator that binds to the carbon source- |
| | responsive element (CSRE) of gluconeogenic genes; involved in the |
| | positive regulation of gluconeogenesis; regulated by Snf1p protein |
| | kinase; localized to the nucleus |
| SPT10 | Putative histone acetylase, sequence-specific activator of histone |
| | genes, binds specifically and highly cooperatively to pairs of UAS |
| | elements in core histone promoters, functions at or near the TATA box |
| MET28 | Basic leucine zipper (bZIP) transcriptional activator in the Cbf1p- |
| | Met4p-Met28p complex, participates in the regulation of sulfur |
| | metabolism |
| GCN4 | Basic leucine zipper (bZIP) transcriptional activator of amino acid |
| | biosynthetic genes in response to amino acid starvation; expression is |
| | tightly regulated at both the transcriptional and translational levels |
| CAD1 | AP-1-like basic leucine zipper (bZIP) transcriptional activator involved |
| | in stress responses, iron metabolism, and pleiotropic drug resistance; |
| | controls a set of genes involved in stabilizing proteins; binds |
| | consensus sequence TTACTAA |
| INO2 | Component of the heteromeric Ino2p/Ino4p basic helix-loop-helix |
| | transcription activator that binds inositol/choline-responsive elements |
| | (ICREs), required for derepression of phospholipid biosynthetic genes |
| | in response to inositol depletion |
| THI2 | Zinc finger protein of the Zn(II)2Cys6 type, probable transcriptional |
| | activator of thiamine biosynthetic genes |
| SWI4 | DNA binding component of the SBF complex (Swi4p-Swi6p), a |
| | transcriptional activator that in concert with MBF (Mbp1-Swi6p) |
| | regulates late G1-specific transcription of targets including cyclins and |
| | genes required for DNA synthesis and repair |
| HAP5 | Subunit of the heme-activated, glucose-repressed Hap2/3/4/5 CCAAT- |
| | binding complex, a transcriptional activator and global regulator of |
| | respiratory gene expression; required for assembly and DNA binding |
| | activity of the complex |
| HAP3 | Subunit of the heme-activated, glucose-repressed Hap2p/3p/4p/5p |
| | CCAAT-binding complex, a transcriptional activator and global |
| | regulator of respiratory gene expression; contains sequences |
| | contributing to both complex assembly and DNA binding |
| HAP2 | Subunit of the heme-activated, glucose-repressed Hap2p/3p/4p/5p |
| | CCAAT-binding complex, a transcriptional activator and global |
| | regulator of respiratory gene expression; contains sequences sufficient |
| | for both complex assembly and DNA binding |
| HAP4 | Subunit of the heme-activated, glucose-repressed Hap2p/3p/4p/5p |
| | CCAAT-binding complex, a transcriptional activator and global |
| | regulator of respiratory gene expression; provides the principal |
| | activation function of the complex |
| YML037C | Putative protein of unknown function with some characteristics of a |
| | transcriptional activator; may be a target of Dbf2p-Mob1p kinase; GFP- |
| | fusion protein co-localizes with clathrin-coated vesicles; YML037C is |
| | not an essential gene |
| TRA1 | Subunit of SAGA and NuA4 histone acetyltransferase complexes; |
| | interacts with acidic activators (e.g., Gal4p) which leads to transcription |
| | activation; similar to human TRRAP, which is a cofactor for c-Myc |
| | mediated oncogenic transformation |
| YLL054C | Putative protein of unknown function with similarity to Pip2p, an oleate- |
| | specific transcriptional activator of peroxisome proliferation; YLL054C |
| | is not an essential gene |
| RTG2 | Sensor of mitochondrial dysfunction; regulates the subcellular location |
| | of Rtg1p and Rtg3p, transcriptional activators of the retrograde (RTG) |
| | and TOR pathways; Rtg2p is inhibited by the phosphorylated form of |
| | Mks1p |
| YBR012C | Dubious open reading frame, unlikely to encode a functional protein; |
| | expression induced by iron-regulated transcriptional activator Aft2p |
| JEN1 | Lactate transporter, required for uptake of lactate and pyruvate; |
| | phosphorylated; expression is derepressed by transcriptional activator |
| | Cat8p during respiratory growth, and repressed in the presence of |
| | glucose, fructose, and mannose |
| MRP1 | Mitochondrial ribosomal protein of the small subunit; MRP1 exhibits |
| | genetic interactions with PET122, encoding a COX3-specific |
| | translational activator, and with PET123, encoding a small subunit |
| | mitochondrial ribosomal protein |
| MRP17 | Mitochondrial ribosomal protein of the small subunit; MRP17 exhibits |
| | genetic interactions with PET122, encoding a COX3-specific |
| | translational activator |
| TPI1 | Triose phosphate isomerase, abundant glycolytic enzyme; mRNA half- |
| | life is regulated by iron availability; transcription is controlled by |
| | activators Reb1p, Gcr1p, and Rap1p through binding sites in the 5′ |
| | non-coding region |
| PKH3 | Protein kinase with similarity to mammalian phosphoinositide- |
| | dependent kinase 1 (PDK1) and yeast Pkh1p and Pkh2p, two |
| | redundant upstream activators of Pkc1p; identified as a multicopy |
| | suppressor of a pkh1 pkh2 double mutant |
| YGL079W | Putative protein of unknown function; green fluorescent protein (GFP)- |
| | fusion protein localizes to the endosome; identified as a transcriptional |
| | activator in a high-throughput yeast one-hybrid assay |
| TFB1 | Subunit of TFIIH and nucleotide excision repair factor 3 complexes, |
| | required for nucleotide excision repair, target for transcriptional |
| | activators |
| PET123 | Mitochondrial ribosomal protein of the small subunit; PET123 exhibits |
| | genetic interactions with PET122, which encodes a COX3 mRNA- |
| | specific translational activator |
| MHR1 | Protein involved in homologous recombination in mitochondria and in |
| | transcription regulation in nucleus; binds to activation domains of acidic |
| | activators; required for recombination-dependent mtDNA partitioning |
| MCM1 | Transcription factor involved in cell-type-specific transcription and |
| | pheromone response; plays a central role in the formation of both |
| | repressor and activator complexes |
| EGD1 | Subunit beta1 of the nascent polypeptide-associated complex (NAC) |
| | involved in protein targeting, associated with cytoplasmic ribosomes; |
| | enhances DNA binding of the Gal4p activator; homolog of human |
| | BTF3b |
| STE5 | Pheromone-response scaffold protein; binds Ste11p, Ste7p, and |
| | Fus3p kinases, forming a MAPK cascade complex that interacts with |
| | the plasma membrane and Ste4p-Ste18p; allosteric activator of Fus3p |
| | that facilitates Ste7p-mediated activation |
| RGT1 | Glucose-responsive transcription factor that regulates expression of |
| | several glucose transporter (HXT) genes in response to glucose; binds |
| | to promoters and acts both as a transcriptional activator and repressor |
| TYE7 | Serine-rich protein that contains a basic-helix-loop-helix (bHLH) DNA |
| | binding motif; binds E-boxes of glycolytic genes and contributes to their |
| | activation; may function as a transcriptional activator in Ty1-mediated |
| | gene expression |
| VMA13 | Subunit H of the eight-subunit V1 peripheral membrane domain of the |
| | vacuolar H+-ATPase (V-ATPase), an electrogenic proton pump found |
| | throughout the endomembrane system; serves as an activator or a |
| | structural stabilizer of the V-ATPase |
| GAL11 | Subunit of the RNA polymerase II mediator complex; associates with |
| | core polymerase subunits to form the RNA polymerase II holoenzyme; |
| | affects transcription by acting as target of activators and repressors |
| VAC14 | Protein involved in regulated synthesis of PtdIns(3,5)P(2), in control of |
| | trafficking of some proteins to the vacuole lumen via the MVB, and in |
| | maintenance of vacuole size and acidity; interacts with Fig4p; activator |
| | of Fab1p |

### Example 6

### Comparison of Entner-Doudoroff Pathway Genes in Yeast Cells

Genomic DNA from Zymomonas mobilis (ZM4) was obtained from the American Type Culture Collection (ATCC accession number 31821 D-5). The genes encoding phosphogluconate dehydratase EC 4.2.1.12 (referred to as “edd”) and 2-keto-3-deoxygluconate-6-phosphate aldolase EC 4.2.1.14 (referred to as “eda”) were isolated from the ZM4 genomic DNA using the following oligonucleotides:

The ZM4 eda gene:

(SEQ ID No: 1)

5′-aactgactagtaaaaaaatgcgtgatatcgattcc-3′

(SEQ ID No: 2)

5′-agtaactcgagctactaggcaacagcagcgcgcttg-3′

The ZM4 edd gene:

(Seq ID No: 3)

5′-aactgactagtaaaaaaatgactgatctgcattcaacg-3′

(Seq ID No: 4)

5′-agtaactcgagctactagataccggcacctgcatatattgc-3′

E. coli genomic DNA was prepared using Qiagen DNeasy blood and tissue kit according to the manufacture's protocol. The E. coli edd and eda constructs were isolated from E. coli genomic DNA using the following oligonucleotides:

The E. coli eda gene:

(SEQ ID NO: 5)

5′-aactgactagtaaaaaaatgaaaaactggaaaacaagtgcagaatc-
3′

(SEQ ID NO: 6)

5′-agtaactcgagctactacagcttagcgccttctacagcttcacg-3′

The E. coli edd gene:

(SEQ ID NO: 7)

5′-aactgactagtaaaaaaatgaatccacaattgttacgcgtaacaaat
cg-3′

(SEQ ID NO: 8)

5′agtaactcgagctactaaaaagtgatacaggttgcgccctgttcggca
c-3′

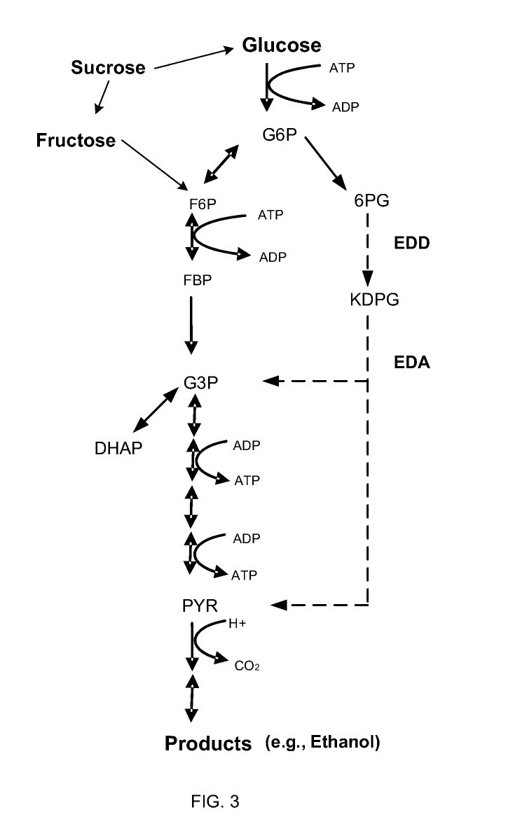

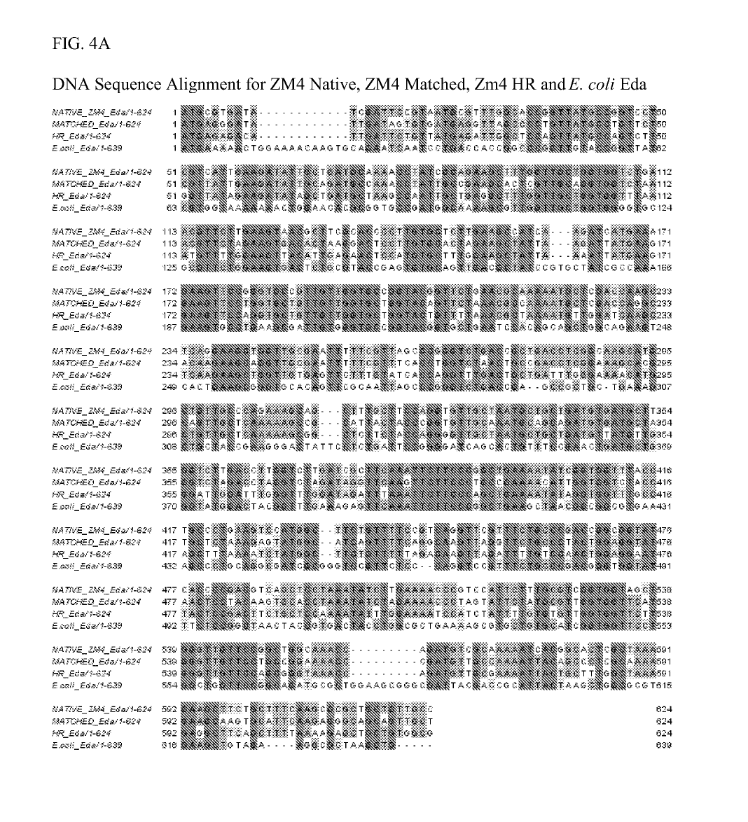

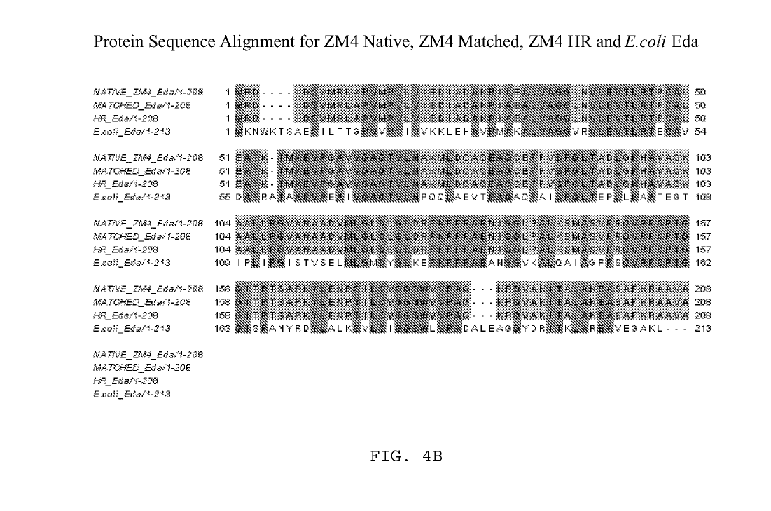

All oligonucleotides set forth above were purchased from Integrated DNA technologies (“IDT”, Coralville, Iowa). These oligonucleotides were designed to incorporate a SpeI restriction endonuclease cleavage site upstream and a XhoI restriction endonuclease cleavage site downstream of the edd and eda gene constructs such that these sites could be used to clone these genes into yeast expression vectors p426GPD (ATCC accession number 87361) and p425GPD (ATCC accession number 87359). In addition to incorporating restriction endonuclease cleavage sites, the forward oligonucleotides were designed to incorporate six consecutive AAAAAA nucleotides immediately upstream of the ATG initiation codon. This ensured that there was a conserved kozak sequence important for efficient translation initiation in yeast. FIG. 3 illustrates a schematic representation of metabolic pathways in an organism engineered to include the EDA and EDD genes.

Cloning the edd and eda genes from ZM4 and E. coli genomic DNA was accomplished using the following procedure: About 100 ng of ZM4 or E. coli genomic DNA, 1 μM of the oligonucleotide primer set listed above, 2.5 U of PfuUltra High-Fidelity DNA polymerase (Stratagene), 300 μM dNTPs (Roche), and 1×PfuUltra reaction buffer was mixed in a final reaction volume of 50 μl. A BIORAD DNA Engine Tetrad 2 Peltier thermal cycler was used for the PCR reactions and the following cycle conditions were used: 5 min denaturation step at 95° C., followed by 30 cycles of 20 sec at 95° C., 20 sec at 55° C., and 1 min at 72° C., and a final step of 5 min at 72° C.

In an attempt to maximize expression of the ZM4 edd and eda genes in yeast, two different approaches were undertaken to optimize the ZM4 edd and eda genes. The first approach was to remove translational pauses from the polynucleotide sequence by designing the gene to incorporate only codons that are preferred in yeast. This optimization is referred to as the “hot rod” optimization. In the second approach, translational pauses which are present in the native organism gene sequence are matched in the heterologous expression host organism by substituting the codon usage pattern of that host organism. This optimization is referred to as the “matched” optimization. The final gene and protein sequences for edd and eda from the ZM4 native, hot rod (HR) and matched versions, as well as the E. coli native are shown in FIG. 6. Certain sequences in FIG. 6 are presented at the end of this Example 1. The matched version of ZM4 edd and ZM4 eda genes were synthesized by IDT, and hot rod versions were constructed using methods described in Larsen et al. (Int. J. Bioinform. Res. Appl; 2008:4[3]; 324-336).

Each version of each edd and eda gene was inserted into the yeast expression vector p426GPD (GPD promoter, 2 micron, URA3) (ATCC accession number 87361) between the SpeI and XhoI cloning sites. Each version of the eda gene was also inserted into the SpeI and XhoI sites of the yeast expression vector p425GPD (GPD promoter, 2 micron, LEU3) (ATCC accession number 87359). For each edd and eda version, 3′ His tagged and non tagged p426 GPD constructs were made. Please refer to table 1 for all oligonucleotides used for PCR amplification of edd and eda constructs for cloning into p425 and p426 GPD vectors. All cloning procedures were conducted according to standard cloning procedures described by Maniatis et al.

Each edd and eda p426GPD construct was transformed into Saccharomyces cerevisiae strain BY4742 (MATalpha his3delta1 leu2delta0 lys2delta0 ura3delta0) (ATCC accession number 201389). This strain has a deletion of the his3 gene, an imidazoleglycerol-phosphate dehydratase which catalyzes the sixth step in histidine biosynthesis; a deletion of leu2 gene, a beta-isopropylmalate dehydrogenase which catalyzes the third step in the leucine biosynthesis pathway; a deletion of the lys2 gene, an alpha aminoadipate reductase which catalyzes the fifth step in biosynthesis of lysine; and a deletion of the ura3 gene, an orotidine-5′-phosphate decarboxylase which catalyzes the sixth enzymatic step in the de novo biosynthesis of pyrimidines. The genotype of BY4742 makes it an auxotroph for histidine, leucine, lysine and uracil.

Transformation of the p426GPD plasmids containing an edd or an eda variant gene into yeast strain BY4742 was accomplished using the Zymo Research frozen-EZ yeast transformation II kit according to the manufacturer's protocol. The transformed BY4742 cells were selected by growth on a synthetic dextrose medium (SD) (0.67% yeast nitrogen base-2% dextrose) containing complete amino acids minus uracil (Krackeler Scientific Inc). Plates were incubated at about 30° C. for about 48 hours. Transformant colonies for each edd and eda variant were inoculated onto 5 ml of SD minus uracil medium and cells were grown at about 30° C. and shaken at about 250 rpm for about 24 hours. Cells were harvested by centrifugation at 1000×g for about 5 minutes, after which protein crude extract was prepared with Y-PER Plus (Thermo Scientific) according to the manufacturer's instructions. Whole cell extract protein concentrations were determined using the Coomassie Plus Protein Assay (Thermo Scientific) according to the manufacturer's directions. For each edd and eda variant His-tagged construct, about 10 μg of soluble and insoluble fractions were loaded on 4-12% NuPAGE Novex Bis-Tris protein gels (Invitrogen) and proteins were analyzed by western using anti-(His)₆ mouse monoclonal antibody (Abcam) and HRP-conjugated secondary antibody (Abcam). Supersignal West Pico Chemiluminescent substrate (Thermo Scientific) was used for western detection according to manufacturer's instructions. All edd variants showed expression in both soluble and insoluble fractions whereas only the E. coli eda variant showed expression in the soluble fraction.

In order to confirm that edd and eda variants were functional in yeast, the combined edd and eda activities were assayed by the formation of pyruvate, coupled to the NADH-dependent activity of lactate dehydrogenase. Transformation of combined edd (in p426GPD) and edd (in p425GPD) constructs was accomplished with the Zymo Research frozen-EZ yeast transformation II kit based on manufacturer's protocol. As a negative control, p425GPD and p426GPD vectors were also transformed into BY4742. Transformants (16 different combinations total including the variant edd and eda combinations plus vector controls) were selected on synthetic dextrose medium (SD) (0.67% yeast nitrogen base-2% dextrose) containing complete amino acids minus uracil and leucine. Transformants of edd and eda variant combinations were inoculated onto 5 ml of SD minus uracil and leucine and cells were grown at about 30° C. in shaker flasks at about 250 rpm for about 24 hours. Fresh overnight culture was used to inoculate about 100 ml of (SD media minus uracil and leucine containing about 0.01 g ergosterol/L and about 400 μl of Tween80) to an initial inoculum OD₆₀₀ₙₘ of about 0.1 and grown anaerobically at about 30° C. for approximately 14 hours until cells reached an OD₆₀₀ₙₘ of 3-4. The cells were centrifuged at about 3000 g for about 10 minutes. The cells were then washed with 25 ml deionized H₂O and centrifuged at 3000 g for 10 min. the cells were resuspended at about 2 ml/g of cell pellet) in lysis buffer (50 mM TrisCl pH7, 10 mM MgCl₂ 1× Calbiochem protease inhibitor cocktail set 111). Approximately 900 μl of glass beads were added and cells were lysed by vortexing at maximum speed for 4×30 seconds. Cell lysate was removed from the glass beads, placed into fresh tubes and spun at about 10,000 g for about 10 minutes at about 4° C. The supernatant containing whole cell extract (WCE) was transferred to a fresh tube. WCE protein concentrations were measured using the Coomassie Plus Protein Assay (Thermo Scientific) according to the manufacturer's directions. A total of about 750 μg of WCE was used for the edd and eda coupled assay. For this assay, about 750 μg of WCE was mixed with about 2 mM 6-phosphogluconate and about 4.5 U lactate dehydrogenase in a final volume of about 400 μl. A total of about 100 μl of NADH was added to this reaction to a final molarity of about 0.3 mM, and NADH oxidation was monitored for about 10 minutes at about 340 nM using a DU800 spectrophotometer.

ZM4 HR EDD GENE
ATGAGAGACATTGATTCTGTTATGAGATTGGCTCCAGTTATGCCAGTCTTGGTTATAGAAGATAT
AGCTGATGCTAAGCCAATTGCTGAGGCTTTGGTTGCTGGTGGTTTAAATGTTTTGGAAGTTACAT
TGAGAACTCCATGTGCTTTGGAAGCTATTAAAATTATGAAGGAAGTTCCAGGTGCTGTTGTTGGT
GCTGGTACTGTTTTAAACGCTAAAATGTTGGATCAAGCTCAAGAAGCTGGTTGTGAGTTCTTTGT
ATCACCAGGTTTGACTGCTGATTTGGGAAAACATGCTGTTGCTCAAAAAGCGGCTCTTCTACCAG
GGGTTGCTAATGCTGCTGATGTTATGTTGGGATTGGATTTGGGTTTGGATAGATTTAAATTCTTC
CCAGCTGAAAATATAGGTGGTTTGCCAGCTTTAAAATCTATGGCTTCTGTTTTTAGACAAGTTAG
ATTTTGTCCAACTGGAGGAATTACTCCGACTTCTGCTCCAAAATATTTGGAAAATCCATCTATTT
TGTGTGTTGGTGGTTCTTGGGTTGTTCCAGCGGGTAAACCAGATGTTGCGAAAATTACTGCTTTG
GCTAAAGAGGCTTCAGCTTTTAAAAGAGCTGCTGTGGCGTAG
ZM4 HR EDD GENE
ATGACGGATTTGCATTCAACTGTTGAGAAAGTAACTGCTAGAGTAATTGAAAGATCAAGGGAAAC
TAGAAAGGCTTATTTGGATTTGATACAATATGAGAGGGAAAAAGGTGTTGATAGACCAAATTTGT
CTTGTTCTAATTTGGCTCATGGTTTTGCTGCTATGAATGGTGATAAACCAGCTTTGAGAGATTTT
AATAGAATGAATATAGGTGTAGTTACTTCTTATAATGATATGTTGTCTGCTCATGAACCATATTA
TAGATATCCAGAACAAATGAAGGTTTTTGCTCGTGAAGTTGGTGCTACAGTTCAAGTTGCTGGTG
GTGTTCCTGCAATGTGTGATGGTGTTACTCAAGGTCAACCAGGTATGGAAGAATCTTTGTTTTCC
AGAGATGTAATTGCTTTGGCTACATCTGTTTCATTGTCTCACGGAATGTTTGAAGGTGCTGCATT
GTTGGGAATTTGTGATAAAATTGTTCCAGGTTTGTTGATGGGTGCTTTGAGGTTCGGTCATTTGC
CAACTATTTTGGTTCCATCTGGTCCAATGACTACTGGAATCCCAAATAAAGAAAAGATTAGAATT
AGACAATTGTATGCTCAAGGAAAAATTGGTCAAAAGGAATTGTTGGATATGGAAGCTGCCTGTTA
TCATGCTGAAGGTACTTGTACTTTTTATGGTACTGCTAACACTAATCAGATGGTTATGGAAGTTT
TGGGTTTGCACATGCCAGGTAGTGCATTCGTTACTCCAGGTACTCCACTGAGACAGGCTTTGACT
AGAGCTGCTGTTCATAGAGTTGCAGAGTTGGGTTGGAAAGGTGATGATTATAGACCTTTGGGTAA
AATTATTGATGAGAAATCTATTGTTAATGCTATTGTTGGTTTGTTAGCTACAGGTGGTTCTACAA
ATCATACAATGCATATTCCGGCCATAGCTAGAGCAGCAGGGGTTATAGTTAATTGGAATGATTTT
CATGATTTGTCTGAAGTTGTTCCATTGATTGCTAGAATTTATCCAAATGGTCCTAGAGATATAAA
TGAATTTCAAAATGCAGGAGGAATGGCTTATGTAATTAAAGAATTGTTGAGTGCGAATTTGTTAA
ATAGAGATGTTACTACTATTGCTAAAGGAGGGATAGAAGAATATGCTAAAGCTCCAGCTCTGAAC
GATGCGGGTGAATTGGTGTGGAAACCGGCTGGCGAACCTGGGGACGACACAATTTTGAGACCAGT
ATCTAATCCATTTGCTAAAGATGGTGGTTTGCGTCTCTTGGAAGGTAATTTGGGTAGAGCAATGT
ATAAGGCTTCTGCTGTAGATCCAAAATTCTGGACTATTGAAGCTCCCGTTAGAGTTTTCTCTGAT
CAAGATGATGTTCAAAAGGCTTTTAAAGCAGGCGAGTTAAATAAAGATGTTATAGTTGTTGTTAG
ATTTCAAGGTCCTCGTGCTAATGGTATGCCTGAATTGCATAAGTTGACTCCTGCGCTAGGCGTAT
TGCAAGATAATGGTTATAAGGTTGCTTTAGTTACTGATGGTAGAATGTCTGGTGCAACTGGTAAA
GTACCGGTGGCTCTGCATGTTTCACCAGAGGCTTTAGGAGGTGGGGCGATTGGCAAGTTGAGAGA
TGGCGATATAGTTAGAATTTCTGTTGAAGAAGGTAAATTAGAGGCTCTTGTCCCCGCCGACGAGT
GGAATGCTAGACCACATGCTGAGAAGCCCGCTTTTAGACCTGGTACTGGGAGAGAATTGTTTGAC
ATTTTTAGACAAAACGCTGCTAAGGCTGAGGATGGTGCAGTTGCAATTTATGCTGGGGCAGGGAT
CTAG
ZM4 MATCHED EDA GENE
ATGAGGGATATTGATAGTGTGATGAGGTTAGCCCCTGTTATGCCTGTTCTCGTTATTGAAGATAT
TGCAGATGCCAAACCTATTGCCGAAGCACTCGTTGCAGGTGGTCTAAACGTTCTAGAAGTGACAC
TAAGGACTCCTTGTGCACTAGAAGCTATTAAGATTATGAAGGAAGTTCCTGGTGCTGTTGTTGGT
GCTGGTACAGTTCTAAACGCCAAAATGCTCGACCAGGCACAAGAAGCAGGTTGCGAATTTTTCGT
TTCACCTGGTCTAACTGCCGACCTCGGAAAGCACGCAGTTGCTCAAAAAGCCGCATTACTACCCG
GTGTTGCAAATGCAGCAGATGTGATGCTAGGTCTAGACCTAGGTCTAGATAGGTTCAAGTTCTTC
CCTGCCGAAAACATTGGTGGTCTACCTGCTCTAAAGAGTATGGCATCAGTTTTCAGGCAAGTTAG
GTTCTGCCCTACTGGAGGTATAACTCCTACAAGTGCACCTAAATATCTAGAAAACCCTAGTATTC
TATGCGTTGGTGGTTCATGGGTTGTTCCTGCCGGAAAACCCGATGTTGCCAAAATTACAGCCCTC
GCAAAAGAAGCAAGTGCATTCAAGAGGGCAGCAGTTGCTTAG
ZM4 MATCHED EDD GENE
ATGACGGATCTACATAGTACAGTGGAGAAGGTTACTGCCAGGGTTATTGAAAGGAGTAGGGAAAC
TAGGAAGGCATATCTAGATTTAATTCAATATGAGAGGGAAAAAGGAGTGGACAGGCCCAACCTAA
GTTGTAGCAACCTAGCACATGGATTCGCCGCAATGAATGGTGACAAGCCCGCATTAAGGGACTTC
AACAGGATGAATATTGGAGTTGTGACGAGTTACAACGATATGTTAAGTGCACATGAACCCTATTA
TAGGTATCCTGAGCAAATGAAGGTGTTTGCAAGGGAAGTTGGAGCCACAGTTCAAGTTGCTGGTG
GAGTGCCTGCAATGTGCGATGGTGTGACTCAGGGTCAACCTGGAATGGAAGAATCCCTATTTTCA
AGGGATGTTATTGCATTAGCAACTTCAGTTTCATTATCACATGGTATGTTTGAAGGGGCAGCTCT
ACTCGGTATATGTGACAAGATTGTTCCTGGTCTACTAATGGGAGCACTAAGGTTTGGTCACCTAC
CTACTATTCTAGTTCCCAGTGGACCTATGACAACGGGTATACCTAACAAAGAAAAAATTAGGATT
AGGCAACTCTATGCACAAGGTAAAATTGGACAAAAAGAACTACTAGATATGGAAGCCGCATGCTA
CCATGCAGAAGGTACTTGCACTTTCTATGGTACAGCCAACACTAACCAGATGGTTATGGAAGTTC
TCGGTCTACATATGCCCGGTAGTGCCTTTGTTACTCCTGGTACTCCTCTCAGGCAAGCACTAACT
AGGGCAGCAGTGCATAGGGTTGCAGAATTAGGTTGGAAGGGAGACGATTATAGGCCTCTAGGTAA
AATTATTGACGAAAAAAGTATTGTTAATGCAATTGTTGGTCTATTAGCCACTGGTGGTAGTACTA
ACCATACGATGCATATTCCTGCTATTGCAAGGGCAGCAGGTGTTATTGTTAACTGGAATGACTTC
CATGATCTATCAGAAGTTGTTCCTTTAATTGCTAGGATTTACCCTAATGGACCTAGGGACATTAA
CGAATTTCAAAATGCCGGAGGAATGGCATATGTTATTAAGGAACTACTATCAGCAAATCTACTAA
ACAGGGATGTTACAACTATTGCTAAGGGAGGTATAGAAGAATACGCTAAGGCACCTGCCCTAAAT
GATGCAGGAGAATTAGTTTGGAAGCCCGCAGGAGAACCTGGTGATGACACTATTCTAAGGCCTGT
TTCAAATCCTTTCGCCAAAGATGGAGGTCTAAGGCTCTTAGAAGGTAACCTAGGAAGGGCCATGT
ACAAGGCTAGCGCCGTTGATCCTAAATTCTGGACTATTGAAGCCCCTGTTAGGGTTTTCTCAGAC
CAGGACGATGTTCAAAAAGCCTTCAAGGCAGGAGAACTAAACAAAGACGTTATTGTTGTTGTTAG
GTTCCAAGGACCTAGGGCCAACGGTATGCCTGAATTACATAAGCTAACTCCTGCATTAGGTGTTC
TACAAGATAATGGATACAAAGTTGCATTAGTGACGGATGGTAGGATGAGTGGTGCAACTGGTAAA
GTTCCTGTTGCATTACATGTTTCACCCGAAGCACTAGGAGGTGGTGCTATTGGTAAACTTAGGGA
TGGAGATATTGTTAGGATTAGTGTTGAAGAAGGAAAACTTGAAGCACTCGTTCCCGCAGATGAGT
GGAATGCAAGGCCTCATGCAGAAAAACCTGCATTCAGGCCTGGGACTGGGAGGGAATTATTTGAT
ATTTTCAGGCAAAATGCAGCAAAAGCAGAAGACGGTGCCGTTGCCATCTATGCCGGTGCTGGTAT
ATAG

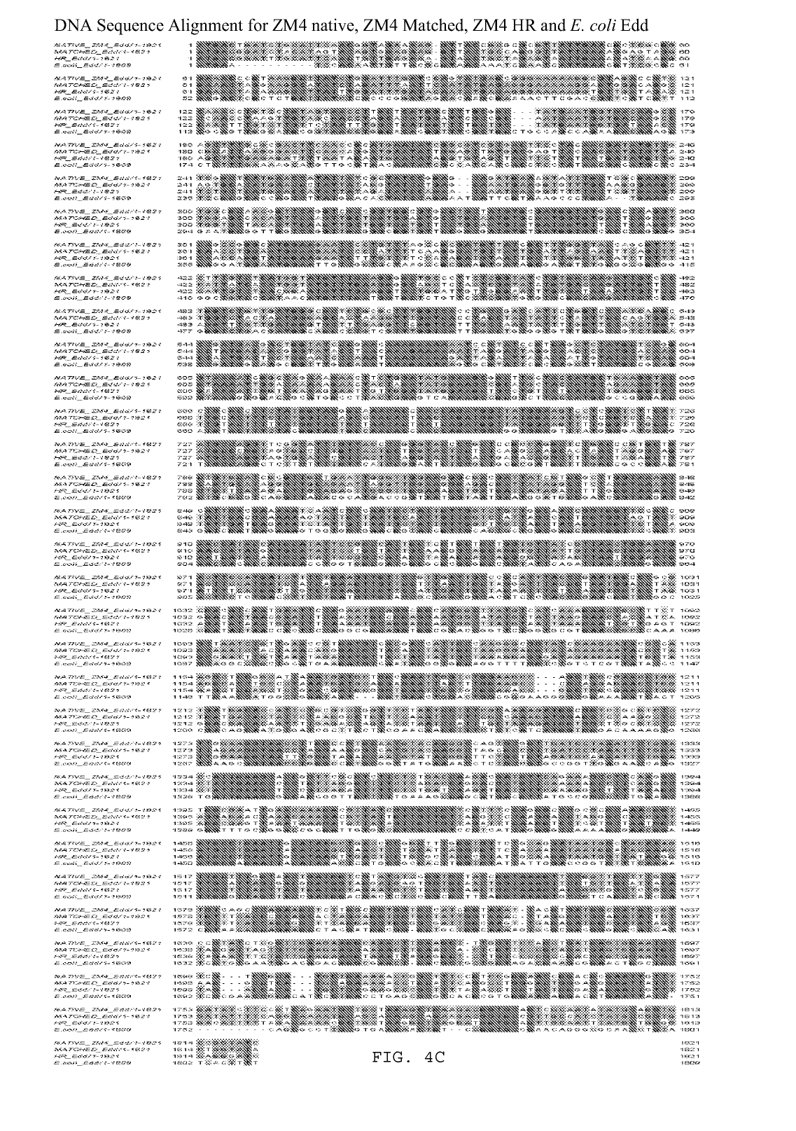

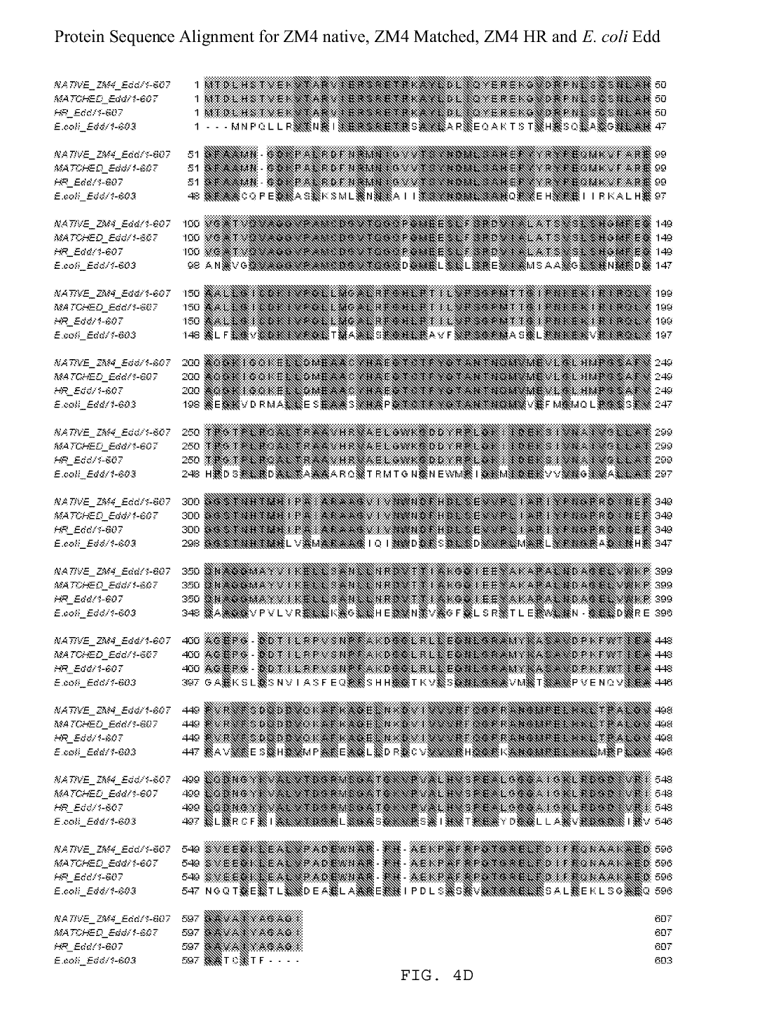

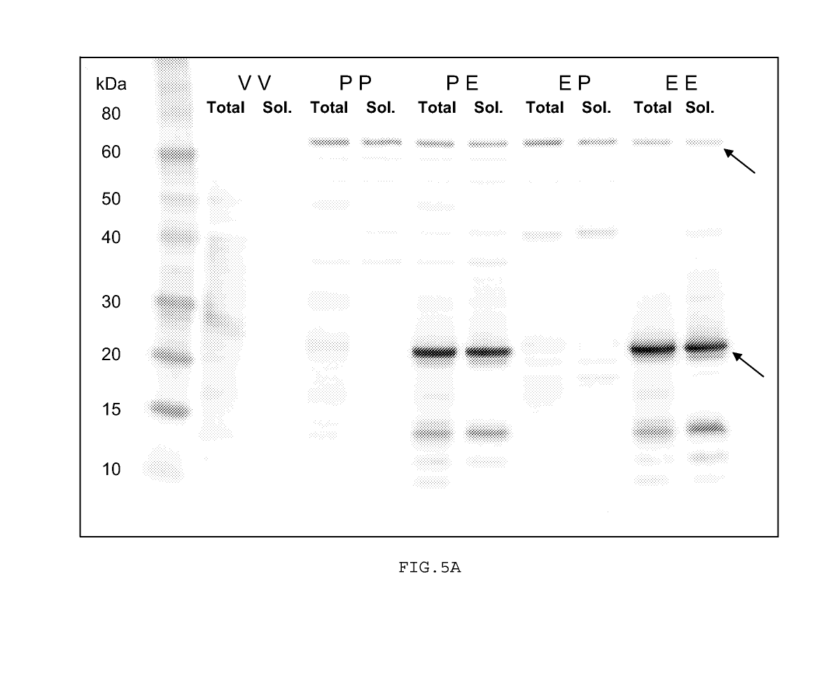

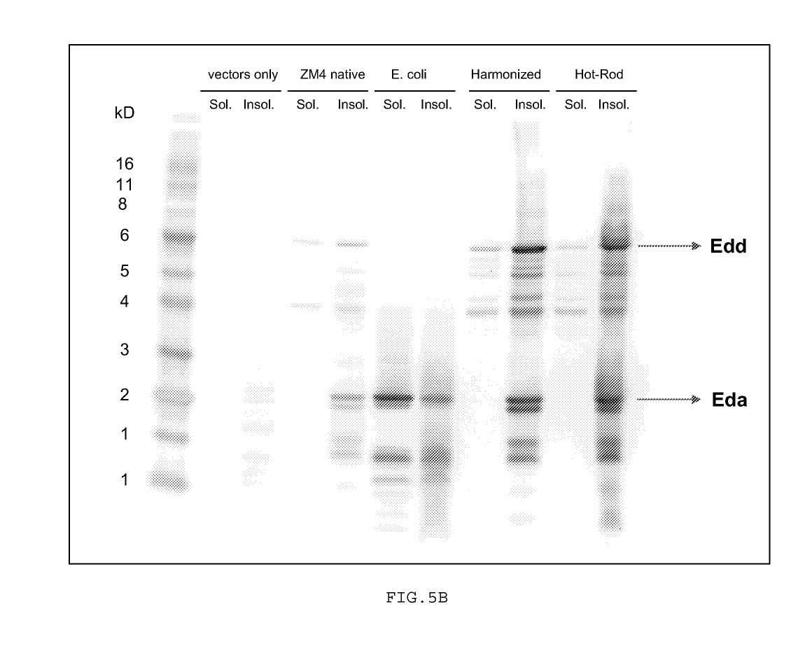

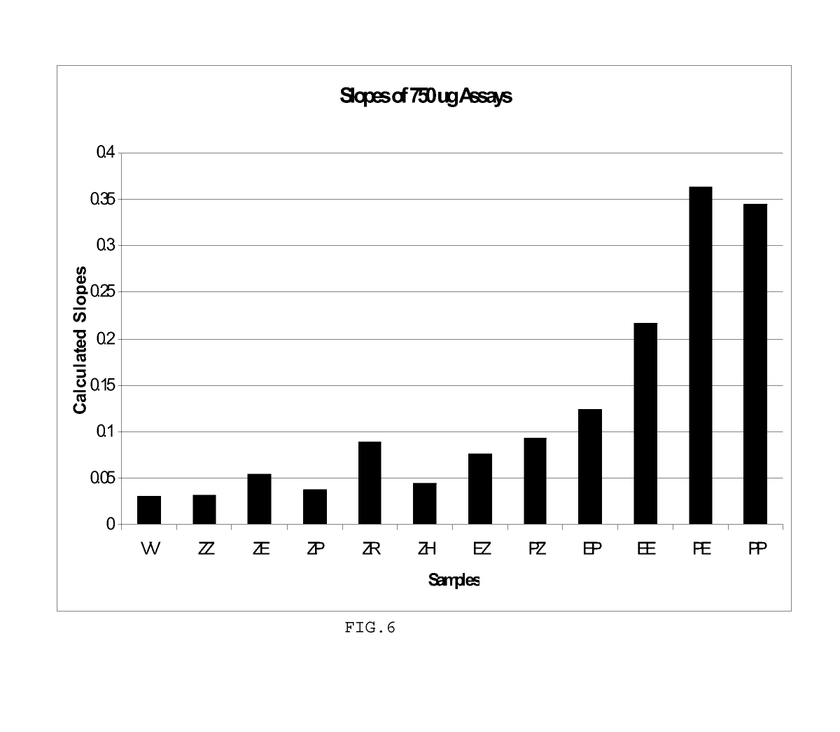

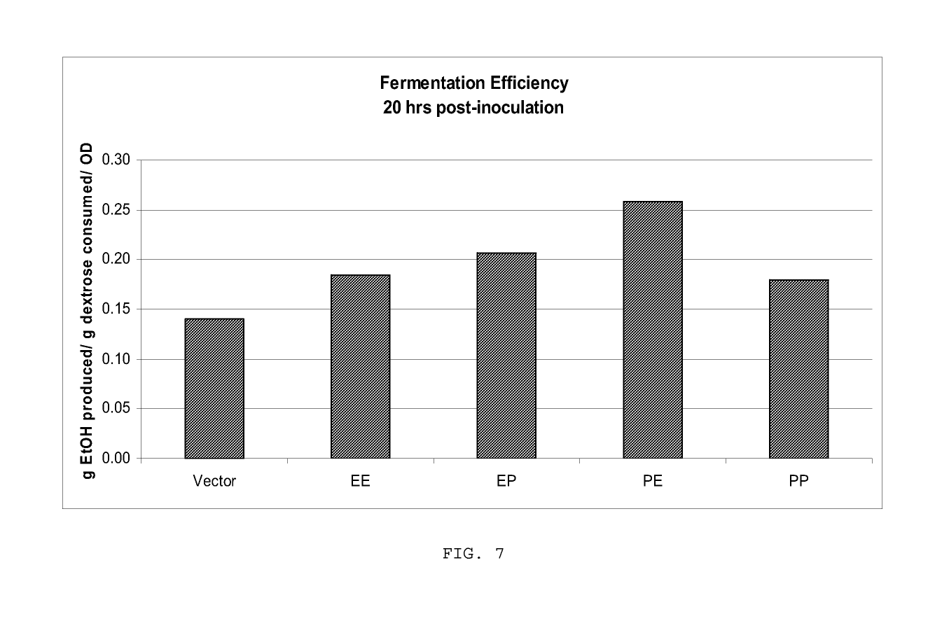

FIGS. 4A-4D show DNA and amino acid sequence alignments for the nucleotide sequences of EDA (FIG. 4A, 4B) and EDD (FIG. 4C, 4D) genes from Zymomonas mobilis (native and optimized) and Escherichia coli.

PCR Amplification of PAO1 Genes

Pseudomonas aeruginosa strain PAO1 DNA was prepared using Qiagen DNeasy Blood and Tissue kit (Qiagen, Valencia, Calif.) according to the manufacture's protocol. The P. aeruginosa edd and eda constructs were isolated from P. aeruginosa genomic DNA using the following oligonucleotides:

The P. aeruginosa edd gene:

(SEQ ID NO: 1)

5′-aactgaactgactagtaaaaaaatgcaccctcgtgtgctcgaagt-
3′

(SEQ ID NO: 2)

5′-agtaaagtaaaagcttctactagcgccagccgttgaggctct-3′

The P. aeruginosa edd gene with 6-HIS c-terminal tag:

(SEQ ID NO: 1)

5′-aactgaactgactagtaaaaaaatgcaccctcgtgtgctcgaagt-
3′

(SEQ ID NO: 3)

5′-agtaaagtaaaagcttctactaatgatgatgatgatgatggcgccag
ccgttgaggctc-3′

The P. aeruginosa eda gene:

(SEQ ID NO: 4)

5′-aactgaactgactagtaaaaaaatgcacaaccttgaacagaagacc-
3′

(SEQ ID NO: 5)

5′-agtaaagtaactcgagctattagtgtctgcggtgctcggcgaa-3′

The P. aeruginosa eda gene with 6-HIS c-terminal tag:

(SEQ ID NO: 6)

5′-aactgaactgactagtaaaaaaatgcacaaccttgaacagaagacc-
3′

(SEQ ID NO: 7)

5′-taaagtaactcgagctactaatgatgatgatgatgatggtgtctgcg
gtgctcggcgaa-3′

P. aeruginosa edd:
SEQ ID NO: 8
ATGCACCCTCGTGTGCTCGAAGTCACCCGCCGCATCCAGGCCCGTAGCGCGGCCACTCGCCAGCG
CTACCTCGAGATGGTCCGGGCTGCGGCCAGCAAGGGGCCGCACCGCGGCACCCTGCCGTGCGGCA
ACCTCGCCCACGGGGTCGCGGCCTGTGGCGAAAGCGACAAGCAGACCCTGCGGCTGATGAACCAG
GCCAACGTGGCCATCGTTTCCGCCTACAACGACATGCTCTCGGCGCACCAGCCGTTCGAGCGCTT
TCCGGGGCTGATCAAGCAGGCGCTGCACGAGATCGGTTCGGTCGGCCAGTTCGCCGGCGGCGTGC
CGGCCATGTGCGACGGGGTGACCCAGGGCGAGCCGGGCATGGAACTGTCGCTGGCCAGCCGCGAC
GTGATCGCCATGTCCACCGCCATCGCGCTGTCTCACAACATGTTCGATGCAGCGCTGTGCCTGGG
TGTTTGCGACAAGATCGTGCCGGGCCTGCTGATCGGCTCGCTGCGCTTCGGCCACCTGCCCACCG
TGTTCGTCCCGGCCGGGCCGATGCCGACCGGCATCTCCAACAAGGAAAAGGCCGCGGTGCGCCAA
CTGTTCGCCGAAGGCAAGGCCACTCGCGAAGAGCTGCTGGCCTCGGAAATGGCCTCCTACCATGC
ACCCGGCACCTGCACCTTCTATGGCACCGCCAATACCAACCAGTTGCTGGTGGAGGTGATGGGCC
TGCACTTGCCCGGTGCCTCCTTCGTCAACCCGAACACCCCCCTGCGCGACGAACTCACCCGCGAA
GCGGCACGCCAGGCCAGCCGGCTGACCCCCGAGAACGGCAACTACGTGCCGATGGCGGAGATCGT
CGACGAGAAGGCCATCGTCAACTCGGTGGTGGCGCTGCTCGCCACCGGCGGCTCGACCAACCACA
CCCTGCACCTGCTGGCGATCGCCCAGGCGGCGGGCATCCAGTTGACCTGGCAGGACATGTCCGAG
CTGTCCCATGTGGTGCCGACCCTGGCGCGCATCTATCCGAACGGCCAGGCCGACATCAACCACTT
CCAGGCGGCCGGCGGCATGTCCTTCCTGATCCGCCAACTGCTCGACGGCGGGCTGCTTCACGAGG
ACGTACAGACCGTCGCCGGCCCCGGCCTGCGCCGCTACACCCGCGAGCCGTTCCTCGAGGATGGC
CGGCTGGTCTGGCGCGAAGGGCCGGAACGGAGTCTCGACGAAGCCATCCTGCGTCCGCTGGACAA
GCCGTTCTCCGCCGAAGGCGGCTTGCGCCTGATGGAGGGCAACCTCGGTCGCGGCGTGATGAAGG
TCTCGGCGGTGGCGCCGGAACACCAGGTGGTCGAGGCGCCGGTACGGATCTTCCACGACCAGGCC
AGCCTGGCCGCGGCCTTCAAGGCCGGCGAGCTGGAGCGCGACCTGGTCGCCGTGGTGCGTTTCCA
GGGCCCGCGGGCGAACGGCATGCCGGAGCTGCACAAGCTCACGCCGTTCCTCGGGGTCCTGCAGG
ATCGTGGCTTCAAGGTGGCGCTGGTCACCGACGGGCGCATGTCCGGGGCGTCGGGCAAGGTGCCC
GCGGCCATCCATGTGAGTCCGGAAGCCATCGCCGGCGGTCCGCTGGCGCGCCTGCGCGACGGCGA
CCGGGTGCGGGTGGATGGGGTGAACGGCGAGTTGCGGGTGCTGGTCGACGACGCCGAATGGCAGG
CGCGCAGCCTGGAGCCGGCGCCGCAGGACGGCAATCTCGGTTGCGGCCGCGAGCTGTTCGCCTTC
ATGCGCAACGCCATGAGCAGCGCGGAAGAGGGCGCCTGCAGCTTTACCGAGAGCCTCAACGGCTG
GCGCTAGTAG
P. aeruginosa edd: Amino Acid

SEQ ID NO: 9

MHPRVLEVTRRIQARSAATRQRYLEMVRAAASKGPHRGTLPCGNLAHGVAACGESDKQTLRLMNQ
ANVAIVSAYNDMLSAHQPFERFPGLIKQALHEIGSVGQFAGGVPAMCDGVTQGEPGMELSLASRD
VIAMSTAIALSHNMFDAALCLGVCDKIVPGLLIGSLRFGHLPTVFVPAGPMPTGISNKEKAAVRQ
LFAEGKATREELLASEMASYHAPGTCTFYGTANTNQLLVEVMGLHLPGASFVNPNTPLRDELTRE
AARQASRLTPENGNYVPMAEIVDEKAIVNSVVALLATGGSTNHTLHLLAIAQAAGIQLTWQDMSE
LSHVVPTLARIYPNGQADINHFQAAGGMSFLIRQLLDGGLLHEDVQTVAGPGLRRYTREPFLEDG
RLVWREGPERSLDEAILRPLDKPFSAEGGLRLMEGNLGRGVMKVSAVAPEHQVVEAPVRIFHDQA
SLAAAFKAGELERDLVAVVRFQGPRANGMPELHKLTPFLGVLQDRGFKVALVTDGRMSGASGKVP
AAIHVSPEAIAGGPLARLRDGDRVRVDGVNGELRVLVDDAEWQARSLEPAPQDGNLGCGRELFAF
MRNAMSSAEEGACSFTESLNGWR
P. aeruginosa eda:
SEQ ID NO. 10
ATGCACAACCTTGAACAGAAGACCGCCCGCATCGACACGCTGTGCCGGGAGGCGCGCATCCTCCC
GGTGATCACCATCGACCGCGAGGCGGACATCCTGCCGATGGCCGATGCCCTCGCCGCCGGCGGCC
TGACCGCCCTGGAGATCACCCTGCGCACGGCGCACGGGCTGACCGCCATCCGGCGCCTCAGCGAG
GAGCGCCCGCACCTGCGCATCGGCGCCGGCACCGTGCTCGACCCGCGGACCTTCGCCGCCGCGGA
AAAGGCCGGGGCGAGCTTCGTGGTCACCCCGGGTTGCACCGACGAGTTGCTGCGCTTCGCCCTGG
ACAGCGAAGTCCCGCTGTTGCCCGGCGTGGCCAGCGCTTCCGAGATCATGCTCGCCTACCGCCAT
GGCTACCGCCGCTTCAAGCTGTTTCCCGCCGAAGTCAGCGGCGGCCCGGCGGCGCTGAAGGCGTT
CTCGGGACCATTCCCCGATATCCGCTTCTGCCCCACCGGAGGCGTCAGCCTGAACAATCTCGCCG
ACTACCTGGCGGTACCCAACGTGATGTGCGTCGGCGGCACCTGGATGCTGCCCAAGGCCGTGGTC
GACCGCGGCGACTGGGCCCAGGTCGAGCGCCTCAGCCGCGAAGCCCTGGAGCGCTTCGCCGAGCA
CCGCAGACACTAATAG
EDA-PAO1 Amino Acid
SEQ ID NO: 11
MHNLEQKTARIDTLCREARILPVITIDREADILPMADALAAGGLTALEITLRTAHGLTAIRRLSE
ERPHLRIGAGTVLDPRTFAAAEKAGASFVVTPGCTDELLRFALDSEVPLLPGVASASEIMLAYRH
GYRRFKLFPAEVSGGPAALKAFSGPFPDIRFCPTGGVSLNNLADYLAVPNVMCVGGTWMLPKAVV
DRGDWAQVERLSREALERFAEHRRH

All oligonucleotides set forth above were purchased from Integrated technologies (“IDT”, Coralville, Iowa). These oligonucleotides were designed to incorporate a SpeI restriction endonuclease cleavage site upstream and either a HindIII restriction endonuclease cleavage site or an XhoI restriction endonuclease cleavage site downstream of the edd and eda gene constructs, respectively such that these sites could be used to clone these genes into yeast expression vectors p426GPD (ATCC accession number 87361) and p425GPD (ATCC accession number 87359). In addition to incorporating restriction endonuclease cleavage sites, the forward oligonucleotides were designed to incorporate six consecutive AAAAAA nucleotides immediately upstream of the ATG initiation codon. This ensured that there was a conserved ribosome binding sequence important for efficient translation initiation in yeast.

PCR amplification of the genes were performed as follows: about 100 ng of the genomic P. aeruginosa PAO1 DNA was added to 1×Pfu Ultra II buffer, 0.3 mM dNTPs, 0.3 μmol gene-specific primers (Seq ID No A-F, combinations as indicated), and 1 U Pfu Ultra II polymerase (Agilent, LaJolla, Calif.) in a 500 reaction mix. This was cycled as follows: 95° C. 10 minutes followed by 30 rounds of 95° C. for 20 seconds, 50° C. (eda amplifications) or 53° C. (edd amplifications) for 30 seconds, and 72° C. for 15 seconds (eda amplifications) or 30 seconds (edd amplifications). A final 5 minute extension reaction at 72° C. was also included. The about 670 bp (eda) or 1830 bp product (edd) was TOPO cloned into the pCR Blunt II TOPO vector (Life Technologies, Carlsbad, Calif.) according to the manufacturer's recommendations.

Cloning of PAO1 edd and eda Genes into Yeast Expression Vectors

Following sequence confirmation (GeneWiz), the about 670 bp SpeI-XhoI eda and about 1830 bp SpeI-HindIII edd fragments were cloned into the corresponding restriction sites in plasmids p425GPD and p426GPD vectors (Mumberg et al., 1995, Gene 156: 119-122; obtained from ATCC #87361; PubMed: 7737504), respectively. Briefly, about 50 ng of SpeI-XhoI-digested p425GPD vector was ligated to about 50 ng of SpeI/XhoI-restricted eda fragment in a 100 reaction with 1×T4 DNA ligase buffer and 1 U T4 DNA ligase (Fermentas) overnight at 16° C. About 30 of this reaction was used to transform DH5α competent cells (Zymo Research) and plated onto LB agar media containing 100 μg/ml ampicillin. Similarly, about 50 ng of SpeI-HindIII-digested p426GPD vector was ligated to about 42 ng of SpeI/HindIII-restricted edd fragment in a 100 reaction with 1×T4 DNA ligase buffer and 1 U T4 DNA ligase (Fermentas) overnight at 16° C. About 3 μl of this reaction was used to transform DH5α competent cells (Zymo Research) and plated onto LB agar media containing 100 μg/ml ampicillin.

A haploid Saccharomyces cerevisiae strain (BY4742; ATCC catalog number 201389) was cultured in YPD media (10 g Yeast Extract, 20 g Bacto-Peptone, 20 g Glucose, 1 L total) at about 30° C. Separate aliquots of these cultured cells were transformed with a plasmid construct(s) containing the eda gene alone, the eda and edd genes, or with vector alone. Transformation was accomplished using the Zymo frozen yeast transformation kit (Catalog number T2001; Zymo Research Corp., Orange, Calif.). To 50 μl of cells was added approximately 0.5-1 μg plasmid DNA and the cells were cultured on drop out media with glucose minus leucine (eda), minus uracil and minus leucine (eda and edd) (about 20 g glucose; about 2.21 g SC drop-out mix [described below], about 6.7 g yeast nitrogen base, all in about 1 L of water); this mixture was cultured for 2-3 days at about 30° C.

SC drop-out mix contained the following ingredients (Sigma); all indicated weights are approximate:

| | |
|---|---|
| 0.4 g | Adenine hemisulfate |
| 3.5 g | Arginine |
| 1 g | Glutamic Acid |
| 0.433 g | Histidine |
| 0.4 g | Myo-Inositol |
| 5.2 g | Isoleucine |
| 2.63 g | Leucine |
| 0.9 g | Lysine |
| 1.5 g | Methionine |
| 0.8 g | Phenylalanine |
| 1.1 g | Serine |
| 1.2 g | Threonine |
| 0.8 g | Tryptophan |
| 0.2 g | Tyrosine |
| 0.2 g | Uracil |
| 1.2 g | Valine |

Activity and Western Analyses

Cell lysates of the various EDD and EDA expressing strains were prepared as follows. About 50 to 100 ml of SCD-ura-leu media containing 10 mM MnCl₂ was used. Aerobically cultured strains were grown in a 250 ml baffled shaker flask. Anaerobically cultured strains were grown in a 250 ml serum bottle outfitted with a butyl rubber stopper with an aluminum crimp cap containing media that included 400 μl/L Tween-80 (British Drug Houses, Ltd., West Chester, Pa.) plus 0.01 g/L Ergosterol (Alef Aesar, Ward Hill, Mass.). Each strain was inoculated at an initial OD₆₀₀ of about 0.2 and grown to an OD₆₀₀ of about 3-4. Cells were grown at 30° C. at 200 rpm.

Yeast cells were harvested by centrifugation at 1046×g (3000 rpm) for 5 minutes at 4° C. The supernatant was discarded and the cells were resuspended and washed twice in 25 mL cold sterile water. Washed cell pellets were resuspended in 1 mL sterile water, transferred to 1.5 mL screw cap tube, and centrifuged at 16,100×g (13,200 rpm) for 3 minutes at 4° C.

Cell pellets were resuspended in about 800 μl-1000 μl of freshly prepared lysis buffer (50 mM Tris-Cl pH 7.0, 10 mM MgCl₂, 1× protease inhibitor cocktail EDTA-free (Thermo Scientific, Waltham, Mass.) and the tube filled with zirconia beads to avoid any headspace in the tube. The tubes were placed in a Mini BeadBeater (Bio Spec Products, Inc., Bartlesville, Okla.) and vortexed twice for 30 seconds at room temperature. The supernatant was transferred to a new 1.5 mL microcentrifuge tube and centrifuged twice to remove cell debris at 16,100×g (13,200 rpm) for 10 minutes, at 4° C. Quantification of the lysates was performed using the Coomassie-Plus kit (Thermo Scientific, San Diego, Calif.) as directed by the manufacturer. The gene combinations contained in each strain is shown in the table below.

| Strain | EDD | EDA |
|---|---|---|
| BF428 | p426GPD (vector control) | p425GPD (vector control) |
| BF604 | E. coli native | E. coli native |
| BF460 | E. coli native with 6-HIS | E. coli native with 6-HIS |
| BF591 | PAO1 native | PAO1 native |
| BF568 | PAO1 native with 6-HIS | PAO1 native with 6-HIS |
| BF592 | PAO1 native | E. coli native |
| BF603 | E. coli native | PAO1 native |

About 5-10 μg of total cell extract was used for SDS-gel [NuPage 4-12% Bis-Tris gels (Life Technologies, Carlsbad, Calif.)] electrophoresis and Western blot analyses. SDS-PAGE gels were run according to the manufacturer's recommendation using NuPage MES-SDS Running Buffer at 1× concentration with the addition of NuPage antioxidant into the cathode chamber at a 1× concentration. Novex Sharp Protein Standards (Life Technologies, Carlsbad, Calif.) were used as standards, as shown in FIGS. 5A and 5B. For Western analysis, gels were transferred onto a nitrocellulose membrane (0.45 micron, Thermo Scientific, San Diego, Calif.) using Western blotting filter paper (Thermo Scientific) using a Bio-Rad Mini Trans-Blot Cell (BioRad, Hercules, Calif.) system for approximately 90 minutes at 40V. Following transfer, the membrane was washed in 1×PBS (EMD, San Diego, Calif.), 0.05% Tween-20 (Fisher Scientific, Fairlawn, N.J.) for 2-5 minutes with gentle shaking. The membrane was blocked in 3% BSA dissolved in 1×PBS and 0.05% Tween-20 at room temperature for about 2 hours with gentle shaking. The membrane was washed once in 1×PBS and 0.05% Tween-20 for about 5 minutes with gentle shaking. The membrane was then incubated at room temperature with a 1:5000 dilution of primary antibody (Ms mAB to 6×His Tag, AbCam, Cambridge, Mass.) in 0.3% BSA (Fraction V, EMD, San Diego, Calif.) dissolved in 1×PBS and 0.05% Tween-20 with gentle shaking for approximately 1 hour. The membrane was washed three times for 5 minutes each with 1×PBS and 0.05% Tween-20 with gentle shaking. The secondary antibody (Dnk pAb to Ms IgG (HRP), AbCam, Cambridge, Mass.] was used at a 1:15000 dilution in 0.3% BSA and allowed to incubate for about 90 minutes at room temperature with gentle shaking. The membrane was washed three times for about 5 minutes using 1×PBS and 0.05% Tween-20, with gentle shaking. The membrane incubated with 5 ml of Supersignal West Pico Chemiluminescent substrate (Thermo Scientific, San Diego, Calif.) for 1 minute and then was exposed to a phosphorimager (Bio-Rad Universal Hood II, Bio-Rad, Hercules, Calif.) for about 10-100 seconds.

The results of the Western blots indicate that both the PAO1 and E. coli EDD proteins are expressed and soluble when expressed in S. cerevisiae, as shown in FIGS. 5A and 5B. The results also demonstrate that the E. coli EDA protein is expressed and soluble. It was not clear if the PAO1 EDA protein is soluble or not in yeast.

### Example 7

### EDD and EDA Activity Assays

Cell lysates of the various EDD and EDA expressing strains were prepared as follows. About 50 to 100 ml of SCD-ura-leu media containing 10 mM MnCl₂ was used. Strains cultured aerobically or anaerobically were grown as described herein (e.g., see Example 6). Each strain was inoculated at an initial OD₆₀₀ of about 0.2 and grown to an OD₆₀₀ of about 3-4. Cells were grown at 30° C. at 200 rpm. Yeast cells were harvested as described in Example 6.

Activity assays were performed as follows: About 750 μg of crude extract, 1× assay buffer (50 mM Tris-Cl pH 7.0, 10 mM MgCl₂), 3 U lactate dehydrogenase (5 μg/μL in 50 mM Tris-Cl pH 7.0), and 10 μl mM 6-phosphogluconate dissolved in 50 mM Tris-Cl pH 7.0 were mixed in a reaction of about 400 μl. The reaction mix was transferred to a 1 ml Quartz cuvette and allowed to incubate about 5 minutes at 30° C. 100 μl of 1.5 mM NADH (prepared in 50 mM Tris-Cl pH 7.0) was added to each reaction mixture, and the change in Abs₃₄₀ₙₘ over a 5 minute time course at 30° C. was monitored in a Beckman DU-800 spectrophotometer using the Enzyme Mechanism software package (Beckman Coulter, Inc, Brea, Calif.). The relative specific activities for BY4742 strains expressing EDD and EDA, from either P. aeruginosa (PAO1) or E. coli sources, are shown in the table below.

| Gene | Km | Vmax | Specific Activity |
|---|---|---|---|
| Combination | (M⁻¹) | (mmol min⁻¹) | (mmol min⁻¹ mg⁻¹) |
| EDD-P/EDA-P | 1.04 × 10⁻³ | 0.21930 | 0.3451 |
| EDD-P/EDA-E | 2.06 × 10⁻³ | 0.27280 | 0.3637 |
| EDD-E/EDA-P | 1.43 × 10⁻³ | 0.09264 | 0.1235 |
| EDD-E/EDA-E | 0.839 × 10⁻³ | 0.16270 | 0.2169 |

These results demonstrate that each of these combinations of EDD and EDA in the S. cerevisiae strain BY4742 confers activity, with the EDD-P/EDA-E and EDD-P/EDA-P combinations conferring the highest level of activity. FIG. 6 graphically represents the relative activities of the various EDD/EDA combinations analyzed herein.

### Example 8

### Improved Ethanol Yield from Yeast Strains Expressing EDD and EDA Constructs

Strains BF428 (vector control), BF591 (EDD-PAO1/EDA-PAO1), BF592 (EDD-PAO1/EDA-E. coli), BF603 (EDD-E. coli/EDA-PAO1) and BF604 (EDD-E. coli/EDA-E. coli) were inoculated into 15 ml SCD-ura-leu media containing 400 μl/L Tween-80 (British Drug Houses, Ltd., West Chester, Pa.) plus 0.01 g/L Ergosterol (EMD, San Diego, Calif.) in 20 ml Hungate tubes outfitted with a butyl rubber stopper and sealed with an aluminum crimped cap to prevent oxygen from entering the culture at an initial OD₆₀₀ of 0.5 and grown for about 20 hours. Glucose and ethanol in the culture media were assayed using YSI 2700 BioAnalyzer instruments (www.ysi.com), according to the manufacturer's recommendations at 0 and 20 hours post inoculation (FIG. B).

FIG. 7 graphically represents the fermentation efficiency of strains carrying various combinations of EDA and EDD genes. The results indicate that the presence of the EDD/EDA combinations in S. cerevisiae increase the yield of ethanol produced as compared to a vector-only control. The labels on the X-axis of FIG. 7 refer to the following gene combinations; Vector=p426GPD/p425GPD; EE=EDD-E. coli/EDA-E. coli, EP=EDD-E. coli/EDA-PAO1; PE=EDD-PAO1/EDA-E. coli, PP=EDD-PAO1/EDA-PAO1.

### Example 9

### Listing of Isolated EDD Genes

| Accession | | Strain | |
|---|---|---|---|
| Number | Species | Number | Nucleotide Sequence |
| YP_526855.1 | Saccharophagus | 2-40 | atgaatagcgtaatcgaagctgtaactcagcgaattattgagcgca |
| | degradans | | gtcgacattctcgtcaggcgtatttgaatttaatgcgcaacaccat |
| | | | ggagcagcatcctcctaaaaagcgtctatcttgcggcaatttggct |
| | | | catgcctatgcagcatgtggtcaatccgataagcaaacaattcgtt |
| | | | taatgcaaagtgcaaacataagtattactacggcatttaacgatat |
| | | | gctttcggcgcatcagcctttagaaacataccctcaaataatcaaa |
| | | | gaaactgcgcgtgcaatgggttcaactgctcaagttgcaggcggcg |
| | | | tgccggcaatgtgtgatggtgtaactcaaggccagcccggtatgga |
| | | | gctgagtttgtttagccgcgaagttgtagcaatggctacagcagta |
| | | | ggcctttcgcacaatatgtttgatggcaatatgtttttgggtgtat |
| | | | gcgataaaattgttcctggcatgctaattggcgcgttgcagtttgg |
| | | | tcatattcctggggtgtttgtgcctgccggaccaatgccttctggt |
| | | | attcccaacaaagaaaaagcaaaagttcgtcagcaatatgcggcgg |
| | | | gcattgtgggggaagataagcttttagaaaccgagtcggcttccta |
| | | | tcacagtgcaggcacgtgtactttttacggtacagcgaatacaaac |
| | | | caaatgatggttgaaatgttgggtgttcagttgcctggctcgtcgt |
| | | | ttgtttaccccggtactgagttgcgtgatgccttaacgagagctgc |
| | | | tgttgaaaagttggtaaaaatcacagattcagccggtaactaccgt |
| | | | ccgctctacgaagtcattacggaaaaatccatcgtcaattcaataa |
| | | | ttggtttgttggctaccggcggttctactaaccacacgctacacat |
| | | | tgttgctgtggctcgcgctgcgggtatagaggttacgtgggcagat |
| | | | atggacgagctttcgcgtgctgtgccattacttgcacgtgtttacc |
| | | | ctaacggcgaagctgatgttaaccaattccagcaggctggcggcat |
| | | | ggcttatttagtaagagagctgcgcagcggcggtttgctaaatgaa |
| | | | gatgtggttactattatgggtgagggcctcgaggcctacgaaaaag |
| | | | agcccatgcttaacgataaggggcaggctgaatgggtaaatgatgt |
| | | | acctgttagccgcgacgataccgttgtgcgtccagttacctcgcct |
| | | | ttcgataaagagggtgggttgcgtctactcaagggtaacttagggc |
| | | | agggcgtaatcaaaatttctgcggtagcgccagaaaatcgcgttgt |
| | | | tgaggccccatgtattgtattcgaggcccaagaagagctaatagct |
| | | | gcgtttaagcgtggtgagctcgaaaaagactttgttgcggtagtgc |
| | | | gcttccaagggccttctgccaatggcatgccagaacttcataaaat |
| | | | gaccccgcctttaggtgtgcttcaagataagggtttcaaggtagcg |
| | | | ttagttaccgatggcagaatgtctggtgcatctggtaaagtgccgg |
| | | | ccggtatacacttgtcgccagaagcgagtaagggtggcctgttgaa |
| | | | taagctgcgcacgggtgatgtgattcgcttcgatgccgaagcgggc |
| | | | gttattcaagcgcttgttagtgatgaagagttagctgcgcgtgagc |
| | | | cagctgtgcaaccggtcgtggagcagaacctcggacgctctctgtt |
| | | | tggtggtttgcgcgatttggctggtgtatcgctacaaggcggaaca |
| | | | gttttcgattttgaaagagagtttggcgaaaaatag |
| NP_642389.1 | Xanthomonas | Pv. | atgagcctgcatccgaatatccaagcCgtcaccgaccgtatccgca |
| | axonopodis | citri | agcgcagTgctccctcgcgcgcggcgtatctggccggCCtcgatgc |
| | | str. | cgccctgcgtgagggcccgttccgtagccggttgagctgcggcaat |
| | | 306 | ctcgcgcatggcttcgctgcgtccgagccGGGcgaCaaatcgcgCc |
| | | | tgcgcggtgcggccacgccgaaCctgggcatcatcactgcctaTaa |
| | | | cgacatgTtgtcggcAcatcagccgttcgagcaCtacccgcagctg |
| | | | atccgCgaaaccgcgcgctcacttggcgccactgcgcaggtggccg |
| | | | gcggcgtgccggcgatgtgtgacggcgtgacccagggccgcgccgg |
| | | | catggagctgtcgctgttctcgcgcgacaacatcgctcaggctgcg |
| | | | gccattggcctgagccatgacatgttcgacagcgtggtgtacctgg |
| | | | gggtgtgcgacaagatcgtgccgggtctgctgatcggtgcgctggc |
| | | | gtttggccatttgccggcgatcttcatgccggctggtccgatgacc |
| | | | ccgggcatcccgaacaagcagaaagccgaagtccgcgaacgctacg |
| | | | ccgctggcgaagccacccgcgccgaattgctggaggccgaatcctc |
| | | | gtcttatcactcgcccggcacctgcaccttttacggcacggcgaac |
| | | | tccaaccaggtgttgctcgaagcgatgggcgtgcagttgcccggcg |
| | | | cctcgttcgtcaatccggagctgccgctgcgcgatgcactgacccg |
| | | | cgaaggcaccgcacgcgcattggcgatctccgcgctgggcgatgac |
| | | | ttccgcccgttcggtcgtttgatcgacgaacgggccatcgtcaatg |
| | | | ccgtggtcgcgctgatggcgaccggcggttcgaccaaccacaccat |
| | | | ccactggatcgcagtggcgcgtgcggccggcatcgtgttgacctgg |
| | | | gacgacatggatctgatctcgcagaccgtgccgctgttgacacgca |
| | | | tctacccgaacggcgaagccgacgtgaaccgcttccaggccgcagg |
| | | | cggcacggcgttcgtgttccgcgaattgatggacgccggctacatg |
| | | | cacgacgacctgccgaccatcgtcgaaggcggcatgcgcgcgtacg |
| | | | tcaacgaaccgcgcctgcaggacggcaaggtgacctacgtgcccgg |
| | | | caccgcgaccactgccgacgacagcgtcgcgcgtccggtcagcgat |
| | | | gcattcgaatcacaaggcggcctgcgcctgctgcgcggcaacctcg |
| | | | gccgctcgttgatcaagctgtcggcggtcaagccgcagcaccgcag |
| | | | catccaagcgccagcggtggtgatcgacaccccgcaagtgctcaac |
| | | | aaactgcatgcggcgggcgtactgccgcacgatttcgtggtggtac |
| | | | tgcgctatcagggcccacgcgcaaacggcatgccggagctgcattc |
| | | | gatggcgccgctactgggcctgctgcagaaccagggccggcgcgtg |
| | | | gcgttggtcaccgacggccgtctgtccggcgcctcgggcaagttcc |
| | | | cggcggcgatccacatgaccccggaagccgcacgcggcggcccgat |
| | | | cgggcgcgtacgcgaaggcgacatcgtgcgactggacggcgaagcc |
| | | | ggcaccttggaagtgctggtttcggccgaagaatgggcatcgcgcg |
| | | | aggtcgcaccgaacactgcgttggccggcaacgacctgggccgcaa |
| | | | cctgttcgccatcaaccgccaggtggttggcccggccgaccagggc |
| | | | gcgatttccatttcctgcggcccgacccatccggacggtgcgctgt |
| | | | ggagctacgacgccgagtacgaactcggtgccgatgcagctgcagc |
| | | | cgccgcgccgcacgagtccaaggacgcctga |
| NP_791117.1 | Pseudomonas | Pv. | atgcatccccgcgtccttgaagtaaccgagcggctcattgctcgca |
| | syringae | tomato | gtcgcgatacccgtcagcgctaccttcaattgattcgaggcgcagc |
| | | str. | gagcgatggcccgatgcgcggcaagcttcaatgtgccaactttgct |
| | | DC3000 | cacggcgtcgccgcctgcggaccggaggacaagcaaagcctgcgtt |
| | | | tgatgaacgccgccaacgtggcaatcgtctcttcctacaatgaaat |
| | | | gctctcggcgcatcagccctacgagcactttcctgcacagatcaaa |
| | | | caggcgttacgtgacattggttcggtcggtcagtttgccggcggcg |
| | | | tgcctgccatgtgcgatggcgtgactcagggtgagccgggcatgga |
| | | | actggccattgccagccgcgaagtgattgccatgtccacggcaatt |
| | | | gccttgtcacacaatatgttcgacgccgccatgatgctgggtatct |
| | | | gcgacaagatcgtccccggcctgatgatgggggcgttgcgtttcgg |
| | | | tcatctgccgaccatcttcgtgccgggcgggccgatggtgtcaggt |
| | | | atctccaacaaggaaaaagccgacgtacggcagcgttacgctgaag |
| | | | gcaaggccagccgtgaagagctgctggactcggaaatgaagtccta |
| | | | tcacggcccgggaacctgcacgttctacggcaccgccaacaccaat |
| | | | cagttggtgatggaagtcatgggcatgcaccttcccggtgcctcgt |
| | | | tcgtcaatccctacacaccactgcgtgatgcgctgacagctgaagc |
| | | | ggctcgtcaggtcacgcgtctgaccatgcaaagcggcagtttcatg |
| | | | ccgattggtgaaatcgtcgacgagcgctcgctggtcaattccatcg |
| | | | ttgcgctgcacgccaccggcggctcgaccaaccacacgctgcacat |
| | | | gccggcgattgctcaggctgcgggtattcagctgacctggcaggac |
| | | | atggccgacctctccgaagtggtgccgaccctcagtcacgtctacc |
| | | | ccaacggcaaggccgacatcaaccatttccaggccgcaggcggcat |
| | | | gtcgttcctgattcgcgagctgctggcagccggtctgctgcacgaa |
| | | | aacgttaacaccgtggccggttatggcctgagccgctacaccaaag |
| | | | agccattcctggaggatggcaaactggtctggcgtgaaggcccgct |
| | | | ggacagcctggatgaaaacatcctgcgcccggtggcgcgtccgttc |
| | | | tcccctgaaggcggtttgcgggtcatggaaggcaacctgggtcgcg |
| | | | gtgtcatgaaagtatcggccgttgcgctggagcatcagattgtcga |
| | | | agcgccagcccgagtgtttcaggatcagaaggagctggccgatgcg |
| | | | ttcaaggccggcgagctggaatgtgatttcgtcgccgtcatgcgtt |
| | | | ttcagggcccgcgctgcaacggcatgcccgaactgcacaagatgac |
| | | | cccgtttctgggcgtgctgcaggatcgtggtttcaaagtggcgctg |
| | | | gtcaccgatggacggatgtcgggcgcctcaggcaagattccggcgg |
| | | | cgattcacgtctgcccggaagcgttcgatggtggcccgttggcact |
| | | | ggtacgcgacggcgatgtgatccgcgtggatggcgtaaaaggcacg |
| | | | ttacaagtgctggtcgaagcgtcagaattggccgcccgagaaccgg |
| | | | ccatcaaccagatcgacaacagtgtcggctgcggtcgcgagctttt |
| | | | tggattcatgcgcatggccttcagctccgcagagcaaggcgccagc |
| | | | gcctttacctctagtctggagacgctcaagtga |
| YP_261706.1 | Pseudomonas | Pf-5 | atgcatccccgcgttcttgaggtcaccgaacggcttatcgcccgta |
| | fluorescens | | gtcgcgccactcgccaggcctatctcgcgctgatccgcgatgccgc |
| | | | cagcgacggcccgcagcggggcaagctgcaatgtgcgaacttcgcc |
| | | | cacggcgtggccggttgcggcaccgacgacaagcacaacctgcgga |
| | | | tgatgaatgcggccaacgtggcaattgtttcgtcatataacgacat |
| | | | gttgtcggcgcaccagccttacgaggtgttccccgagcagatcaag |
| | | | cgcgccctgcgcgagatcggctcggtgggccagttcgccggcggca |
| | | | ccccggccatgtgcgatggcgtgacccagggcgaggccggtatgga |
| | | | actgagcctgccgagccgtgaagtgatcgccctgtctacggcggtg |
| | | | gccctctctcacaacatgttcgatgccgcgctgatgctggggatct |
| | | | gcgacaagattgtcccggggttgatgatgggcgctctgcgcttcgg |
| | | | tcacctgccgaccatcttcgttccgggcgggcccatggtctcgggc |
| | | | atttccaacaagcagaaagccgacgtgcgccagcgttacgccgaag |
| | | | gcaaggccagccgcgaggaactgctggagtcggaaatgaagtccta |
| | | | ccacagccccggcacctgcactttctacggcaccgccaacaccaac |
| | | | cagttgctgatggaagtgatgggcctgcacctgccgggcgcctctt |
| | | | tcgtcaaccccaatacgccgctgcgcgacgccctgacccatgaggc |
| | | | ggcgcagcaggtcacgcgcctgaccaagcagagcggggccttcatg |
| | | | ccgattggcgagatcgtcgacgagcgcgtgctggtcaactccatcg |
| | | | ttgccctgcacgccacgggcggctccaccaaccacaccctgcacat |
| | | | gccggccatcgcccaggcggcgggcatccagctgacctggcaggac |
| | | | atggccgacctctccgaggtggtgccgaccctgtcccacgtctatc |
| | | | caaacggcaaggccgatatcaaccacttccaggcggcgggcggcat |
| | | | gtctttcctgatccgcgagctgctggaagccggcctgctccacgaa |
| | | | gacgtcaataccgtggccggccgcggcctgagccgctatacccagg |
| | | | aacccttcctggacaacggcaagctggtgtggcgcgacggcccgat |
| | | | tgaaagcctggacgaaaacatcctgcgcccggtggcccgggcgttc |
| | | | tctgcggagggcggcttgcgggtcatggaaggcaacctcggtcgcg |
| | | | gcgtgatgaaggtttccgccgtggccccggagcaccagatcgtcga |
| | | | ggccccggccgtggtgttccaggaccagcaggacctggccgatgcc |
| | | | ttcaaggccggcctgctggagaaggacttcgtcgcggtgatgcgct |
| | | | tccagggcccgcgctccaacggcatgcccgagctgcacaagatgac |
| | | | ccccttcctcggggtgctgcaggaccgcggcttcaaggtggcgctg |
| | | | gtcaccgacgggcgcatgtccggcgcttcgggcaagattccggcag |
| | | | cgatccatgtcagccccgaagcccaggtgggtggcgcgctggcccg |
| | | | ggtgctggacggcgatatcatccgagtggatggcgtcaagggcacc |
| | | | ctggagcttaaggtagacgccgcagaattcgccgcccgggagccgg |
| | | | ccaagggcctgctgggcaacaacgttggcaccggccgcgaactctt |
| | | | cgccttcatgcgcatggccttcagctcggcagagcagggcgccagc |
| | | | gcctttacctctgccctggagacgctcaagtga |
| ZP_0359148.1 | Bacillus | Subtilis | atggcagaattacgcagtaatatgatcacacaaggaatcgatagag |
| | Subtilis | str. | ctccgcaccgcagtttgcttcgtgcagcaggggtaaaagaagagga |
| | | 168 | tttcggcaagccgtttattgcggtgtgtaattcatacattgatatc |
| | | | gttcccggtcatgttcacttgcaggagtttgggaaaatcgtaaaag |
| | | | aagcaatcagagaagcagggggcgttccgtttgaatttaataccat |
| | | | tggggtagatgatggcatcgcaatggggcatatcggtatgagatat |
| | | | tcgctgccaagccgtgaaattatcgcagactctgtggaaacggttg |
| | | | tatccgcacactggtttgacggaatggtctgtattccgaactgcga |
| | | | caaaatcacaccgggaatgcttatggcggcaatgcgcatcaacatt |
| | | | ccgacgatttttgtcagcggcggaccgatggcggcaggaagaacaa |
| | | | gttacgggcgaaaaatctccctttcctcagtattcgaaggggtagg |
| | | | cgcctaccaagcagggaaaatcaacgaaaacgagcttcaagaacta |
| | | | gagcagttcggatgcccaacgtgcgggtcttgctcaggcatgttta |
| | | | cggcgaactcaatgaactgtctgtcagaagcacttggtcttgcttt |
| | | | gccgggtaatggaaccattctggcaacatctccggaacgcaaagag |
| | | | tttgtgagaaaatcggctgcgcaattaatggaaacgattcgcaaag |
| | | | atatcaaaccgcgtgatattgttacagtaaaagcgattgataacgc |
| | | | gtttgcactcgatatggcgctcggaggttctacaaataccgttctt |
| | | | catacccttgcccttgcaaacgaagccggcgttgaatactctttag |
| | | | aacgcattaacgaagtcgctgagcgcgtgccgcacttggctaagct |
| | | | ggcgcctgcatcggatgtgtttattgaagatcttcacgaagcgggc |
| | | | ggcgtttcagcggctctgaatgagctttcgaagaaagaaggagcgc |
| | | | ttcatttagatgcgctgactgttacaggaaaaactcttggagaaac |
| | | | cattgccggacatgaagtaaaggattatgacgtcattcacccgctg |
| | | | gatcaaccattcactgaaaagggaggccttgctgttttattcggta |
| | | | atctagctccggacggcgctatcattaaaacaggcggcgtacagaa |
| | | | tgggattacaagacacgaagggccggctgtcgtattcgattctcag |
| | | | gacgaggcgcttgacggcattatcaaccgaaaagtaaaagaaggcg |
| | | | acgttgtcatcatcagatacgaagggccaaaaggcggacctggcat |
| | | | gccggaaatgctggcgccaacatcccaaatcgttggaatgggactc |
| | | | gggccaaaagtggcattgattacggacggacgtttttccggagcct |
| | | | cccgtggcctctcaatcggccacgtatcacctgaggccgctgaggg |
| | | | cgggccgcttgcctttgttgaaaacggagaccatattatcgttgat |
| | | | attgaaaaacgcatcttggatgtacaagtgccagaagaagagtggg |
| | | | aaaaacgaaaagcgaactggaaaggttttgaaccgaaagtgaaaac |
| | | | cggctacctggcacgttattctaaacttgtgacaagtgccaacacc |
| | | | ggcggtattatgaaaatctag |
| YP_091897.1 | Bacillus | ATCC | atgacaggtttacgcagtgacatgattacaaaagggatcgacagag |
| | licheniformis | 14580 | cgccgcaccgcagtttgctgcgcgcggctggggtaaaagaagagga |
| | | | cttcggcaaaccgtttattgccgtttgcaactcatacatcgatatc |
| | | | gtaccgggtcatgtccatttgcaggagtttggaaaaatcgtcaaag |
| | | | aggcgatcagagaggccggcggtgttccgtttgaatttaatacaat |
| | | | cggggtcgacgacggaattgcgatggggcacatcggaatgaggtat |
| | | | tctctcccgagccgcgaaatcatcgcagattcagtggaaacggttg |
| | | | tatcggcgcactggtttgacggaatggtatgtattccaaactgtga |
| | | | taaaatcacaccgggcatgatcatggcggcaatgcggatcaacatt |
| | | | ccgaccgtgtttgtcagcggggggccgatggaagcgggaagaacga |
| | | | gcgacggacgaaaaatctcgctttcctctgtatttgaaggcgttgg |
| | | | cgcttatcaatcaggcaaaatcgatgagaaaggactcgaggagctt |
| | | | gaacagttcggctgtccgacttgcggatcatgctcgggcatgttta |
| | | | cggcgaactcgatgaactgtctttctgaagctcttggcatcgccat |
| | | | gccgggcaacggcaccattttggcgacatcgcccgaccgcagggaa |
| | | | tttgccaaacagtcggcccgccagctgatggagctgatcaagtcgg |
| | | | atatcaaaccgcgcgacatcgtgaccgaaaaagcgatcgacaacgc |
| | | | gttcgctttagacatggcgctcggcggatcaacgaatacgatcctt |
| | | | catacgcttgcgatcgccaatgaagcgggtgtagactattcgcttg |
| | | | aacggatcaatgaggtagcggcaagggttccgcatttatcgaagct |
| | | | tgcaccggcttccgatgtgtttattgaagatttgcatgaagcagga |
| | | | ggcgtatcggcagtcttaaacgagctgtcgaaaaaagaaggcgcgc |
| | | | ttcacttggatacgctgactgtaacggggaaaacgcttggcgaaaa |
| | | | tattgccggacgcgaagtgaaagattacgaggtcattcatccgatc |
| | | | gatcagccgttttcagagcaaggcggactcgccgtcctgttcggca |
| | | | acctggctcctgacggtgcgatcattaaaacgggcggcgtccaaga |
| | | | cgggattacccgccatgaaggacctgcggttgtctttgattcacag |
| | | | gaagaagcgcttgacggcatcatcaaccgtaaagtaaaagcgggag |
| | | | atgtcgtcatcatccgctatgaaggccctaaaggcggaccgggaat |
| | | | gcctgaaatgcttgcgccgacttcacagatcgtcggaatgggcctc |
| | | | ggcccgaaagtcgccttgattaccgacggccgcttttcaggagcct |
| | | | cccgcggtctttcgatcggccacgtttcaccggaagcagccgaagg |
| | | | cggcccgcttgctttcgtagaaaacggcgaccatatcgttgtcgat |
| | | | atcgaaaagcggattttaaacatcgaaatctccgatgaggaatggg |
| | | | aaaaaagaaaagcaaactggcccggctttgaaccgaaagtgaaaac |
| | | | gggctatctcgccaggtattcaaagcttgtgacatctgccaatacc |
| | | | ggcggcattatgaaaatctag |
| NP_0718074.1 | Sewanella | MR-1 | atgcactcagtcgttcaatctgttactgacagaattattgcccgta |
| | oneidensis | | gcaaagcatctcgtgaagcataccttgctgcgttaaacgatgcccg |
| | | | taaccatggtgtacaccgaagttccttaagttgcggtaacttagcc |
| | | | cacggttttgcggcttgtaatcccgatgacaaaaatgcattgcgtc |
| | | | aattgacgaaggccaatattgggattatcaccgcattcaacgatat |
| | | | gttatctgcacaccaaccctatgaaacctatcctgatttgctgaaa |
| | | | aaagcctgtcaggaagtcggtagtgttgcgcaggtggctggcggtg |
| | | | ttcccgccatgtgtgacggcgtgactcaaggtcagcccggtatgga |
| | | | attgagcttactgagccgtgaagtgattgcgatggcaaccgcggtt |
| | | | ggcttatcacacaatatgtttgatggagccttactcctcggtattt |
| | | | gcgataaaattgtaccgggtttactgattggtgccttaagttttgg |
| | | | ccatttacctatgttgtttgtgcccgcaggcccaatgaaatcgggt |
| | | | attcctaataaggaaaaagctcgcattcgtcagcaatttgctcaag |
| | | | gtaaggtcgatagagcacaactgctcgaagcggaagcccagtctta |
| | | | ccacagtgcgggtacttgtaccttctatggtaccgctaactcgaac |
| | | | caactgatgctcgaagtgatggggctgcaattgccgggttcatctt |
| | | | ttgtgaatccagacgatccactgcgcgaagccttaaacaaaatggc |
| | | | ggccaagcaggtttgtcgtttaactgaactaggcactcaatacagt |
| | | | ccgattggtgaagtcgttaacgaaaaatcgatagtgaatggtattg |
| | | | ttgcattgctcgcgacgggtggttcaacaaacttaaccatgcacat |
| | | | tgtggcggcggcccgtgctgcaggtattatcgtcaactgggatgac |
| | | | ttttcggaattatccgatgcggtgcctttgctggcacgtgtttatc |
| | | | caaacggtcatgcggatattaaccatttccacgctgcgggtggtat |
| | | | ggctttccttatcaaagaattactcgatgcaggtttgctgcatgag |
| | | | gatgtcaatactgtcgcgggttatggtctgcgccgttacacccaag |
| | | | agcctaaactgcttgatggcgagctgcgctgggtcgatggcccaac |
| | | | agtgagtttagataccgaagtattaacctctgtggcaacaccattc |
| | | | caaaacaacggtggtttaaagctgctgaagggtaacttaggccgcg |
| | | | ctgtgattaaagtgtctgccgttcagccacagcaccgtgtggtgga |
| | | | agcgcccgcagtggtgattgacgatcaaaacaaactcgatgcgtta |
| | | | tttaaatccggcgcattagacagggattgtgtggtggtggtgaaag |
| | | | gccaagggccgaaagccaacggtatgccagagctgcataaactaac |
| | | | gccgctgttaggttcattgcaggacaaaggctttaaagtggcactg |
| | | | atgactgatggtcgtatgtcgggcgcatcgggcaaagtacctgcgg |
| | | | cgattcatttaacccctgaagcgattgatggcgggttaattgcaaa |
| | | | ggtacaagacggcgatttaatccgagttgatgcactgaccggcgag |
| | | | ctgagtttattagtctctgacaccgagcttgccaccagaactgcca |
| | | | ctgaaattgatttacgccattctcgttatggcatggggcgtgagtt |
| | | | atttggagtactgcgttcaaacttaagcagtcctgaaaccggtgcg |
| | | | cgtagtactagcgccatcgatgaactttactaa |
| YP_190870.1 | Gluconobacter | 621H | atgtctctgaatcccgtcgtcgagagcgtgactgcccgtatcatcg |
| | oxydans | | agcgttcgaaagtctcccgtcgccggtatctcgccctgatggagcg |
| | | | caaccgcgccaagggtgtgctccggcccaagctggcctgcggtaat |
| | | | ctggcgcatgccatcgcagcgtccagccccgacaagccggatctga |
| | | | tgcgtcccaccgggaccaatatcggcgtgatcacgacctataacga |
| | | | catgctctcggcgcatcagccgtatggccgctatcccgagcagatc |
| | | | aagctgttcgcccgtgaagtcggtgcgacggcccaggttgcaggcg |
| | | | gcgcaccagcaatgtgtgatggtgtgacgcaggggcaggagggcat |
| | | | ggaactctccctgttctcccgtgacgtgatcgccatgtccacggcg |
| | | | gtcgggctgagccacggcatgtttgagggcgtggcgctgctgggca |
| | | | tctgtgacaagattgtgccgggccttctgatgggcgcgctgcgctt |
| | | | cggtcatctcccggccatgctgatcccggcagggccaatgccgtcc |
| | | | ggtcttccaaacaaggaaaagcagcgcatccgccagctctatgtgc |
| | | | agggcaaggtcgggcaggacgagctgatggaagcggaaaacgcctc |
| | | | ctatcacagcccgggcacctgcacgttctatggcacggccaatacg |
| | | | aaccagatgatggtcgaaatcatgggtctgatgatgccggactcgg |
| | | | ctttcatcaatcccaacacgaagctgcgtcaggcaatgacccgctc |
| | | | gggtattcaccgtctggccgaaatcggcctgaacggcgaggatgtg |
| | | | cgcccgctcgctcattgcgtagacgaaaaggccatcgtgaatgcgg |
| | | | cggtcgggttgctggcgacgggtggttcgaccaaccattcgatcca |
| | | | tcttcctgctatcgcccgtgccgctggtatcctgatcgactgggaa |
| | | | gacatcagccgcctgtcgtccgcggttccgctgatcacccgtgttt |
| | | | atccgagcggttccgaggacgtgaacgcgttcaaccgcgtgggtgg |
| | | | tatgccgaccgtgatcgccgaactgacgcgcgccgggatgctgcac |
| | | | aaggacattctgacggtctctcgtggcggtttctccgattatgccc |
| | | | gtcgcgcatcgctggaaggcgatgagatcgtctacacccacgcgaa |
| | | | gccgtccacggacaccgatatcctgcgcgatgtggctacgcctttc |
| | | | cggcccgatggcggtatgcgcctgatgactggtaatctgggccgcg |
| | | | cgatctacaagagcagcgctattgcgcccgagcacctgaccgttga |
| | | | agcgccggcacgggtcttccaggaccagcatgacgtcctcacggcc |
| | | | tatcagaatggtgagcttgagcgtgatgttgtcgtggtcgtccggt |
| | | | tccagggaccggaagccaacggcatgccggagcttcacaagctgac |
| | | | cccgactctgggcgtgcttcaggatcgcggcttcaaggtggccctg |
| | | | ctgacggatggacgcatgtccggtgcgagcggcaaggtgccggccg |
| | | | ccattcatgtcggtcccgaagcgcaggttggcggtccgatcgcccg |
| | | | cgtgcgggacggcgacatgatccgtgtctgcgcggtgacgggacag |
| | | | atcgaggctctggtggatgccgccgagtgggagagccgcaagccgg |
| | | | tcccgccgccgctcccggcattgggaacgggccgcgaactgttcgc |
| | | | gctgatgcgttcggtgcatgatccggccgaggctggcggatccgcg |
| | | | atgctggcccagatggatcgcgtgatcgaagccgttggcgacgaca |
| | | | ttcactaa |
| ZP_06145432.1 | Ruminococcus | FD-1 | atgagcgataattttttctgcgagggtgcggataaagcccctcagc |
| | flavefaciens | | gttcacttttcaatgcactgggcatgactaaagaggaaatgaagcg |
| | | | tcccctcgttggtatcgtttcttcctacaatgagatcgttcccggc |
| | | | catatgaacatcgacaagctggtcgaagccgttaagctgggtgtag |
| | | | ctatgggcggcggcactcctgttgttttccctgctatcgctgtatg |
| | | | cgacggtatcgctatgggtcacacaggcatgaagtacagccttgtt |
| | | | acccgtgaccttattgccgattctacagagtgtatggctcttgctc |
| | | | atcacttcgacgcactggtaatgatacctaactgcgacaagaacgt |
| | | | tcccggcctgcttatggcggctgcacgtatcaatgttcctactgta |
| | | | ttcgtaagcggcggccctatgcttgcaggccatgtaaagggtaaga |
| | | | agacctctctttcatccatgttcgaggctgtaggcgcttacacagc |
| | | | aggcaagatagacgaggctgaacttgacgaattcgagaacaagacc |
| | | | tgccctacctgcggttcatgttcgggtatgtataccgctaactcca |
| | | | tgaactgcctcactgaggtactgggtatgggtctcagaggcaacgg |
| | | | cactatccctgctgtttactccgagcgtatcaagcttgcaaagcag |
| | | | gcaggtatgcaggttatggaactctacagaaagaatatccgccctc |
| | | | tcgatatcatgacagagaaggctttccagaacgctctcacagctga |
| | | | tatggctcttggatgttccacaaacagtatgctccatctccctgct |
| | | | atcgccaacgaatgcggcataaatatcaaccttgacatggctaacg |
| | | | agataagcgccaagactcctaacctctgccatcttgcaccggcagg |
| | | | ccacacctacatggaagacctcaacgaagcaggcggagtttatgca |
| | | | gttctcaacgagctgagcaaaaagggacttatcaacaccgactgca |
| | | | tgactgttacaggcaagaccgtaggcgagaatatcaagggctgcat |
| | | | caaccgtgaccctgagactatccgtcctatcgacaacccatacagt |
| | | | gaaacaggcggaatcgccgtactcaagggcaatcttgctcccgaca |
| | | | gatgtgttgtgaagagaagcgcagttgctcccgaaatgctggtaca |
| | | | caaaggccctgcaagagtattcgacagcgaggaagaagctatcaag |
| | | | gtcatctatgagggcggtatcaaggcaggcgacgttgttgttatcc |
| | | | gttacgaaggccctgcaggcggccccggcatgagagaaatgctctc |
| | | | tcctacatcagctatacagggtgcaggtctcggctcaactgttgct |
| | | | ctaatcactgacggacgtttcagcggcgctacccgtggtgcggcta |
| | | | tcggacacgtatcccccgaagctgtaaacggcggtactatcgcata |
| | | | tgtcaaggacggcgatattatctccatcgacataccgaattactcc |
| | | | atcactcttgaagtatccgacgaggagcttgcagagcgcaaaaagg |
| | | | caatgcctatcaagcgcaaggagaacatcacaggctatctgaagcg |
| | | | ctatgcacagcaggtatcatccgcagacaagggcgctatcatcaac |
| | | | aggaaatag |

| Accession | | Strain | |
|---|---|---|---|
| Number | Species | Number | Amino Acid Sequence |
| YP_526855.1 | Saccharophagus | 2-40 | MNSVIEAVTQRIIERSRHSRQA |
| | degradans | | YLNLMRNTMEQHPPKKRLSCGN |
| | | | LAHAYAACGQSDKQTIRLMQSA |
| | | | NISITTAFNDMLSAHQPLETYP |
| | | | QIIKETARAMGSTAQVAGGVPA |
| | | | MCDGVTQGQPGMELSLFSREVV |
| | | | AMATAVGLSHNMFDGNMFLGVC |
| | | | DKIVPGMLIGALQFGHIPGVFV |
| | | | PAGPMPSGIPNKEKAKVRQQYA |
| | | | AGIVGEDKLLETESASYHSAGT |
| | | | CTFYGTANTNQMMVEMLGVQLP |
| | | | GSSFVYPGTELRDALTRAAVEK |
| | | | LVKITDSAGNYRPLYEVITEKS |
| | | | IVNSIIGLLATGGSTNHTLHIV |
| | | | AVARAAGIEVTWADMDELSRAV |
| | | | PLLARVYPNGEADVNQFQQAGG |
| | | | MAYLVRELRSGGLLNEDVVTIM |
| | | | GEGLEAYEKEPMLNDKGQAEWV |
| | | | NDVPVSRDDTVVRPVTSPFDKE |
| | | | GGLRLLKGNLGQGVIKISAVAP |
| | | | ENRVVEAPCIVFEAQEELIAAF |
| | | | KRGELEKDFVAVVRFQGPSANG |
| | | | MPELHKMTPPLGVLQDKGFKVA |
| | | | LVTDGRMSGASGKVPAGIHLSP |
| | | | EASKGGLLNKLRTGDVIRFDAE |
| | | | AGVIQALVSDEELAAREPAVQP |
| | | | VVEQNLGRSLFGGLRDLAGVSL |
| | | | QGGTVFDFEREFGEK |
| NP_642389.1 | Xanthomonas | Pv. | MSLHPNIQAVTDRIRKRSAPSR |
| | axonopodis | citri | AAYLAGIDAALREGPFRSRLSC |
| | | str. | GNLAHGFAASEPTDKSRLRGAA |
| | | 306 | TPNLGIITAYNDMLSAHQPFEH |
| | | | YPQLIRETARSLGATAQVAGGV |
| | | | PAMCDGVTQGRAGMELSLFSRD |
| | | | NIAQAAAIGLSHDMFDSVVYLG |
| | | | VCDKIVPGLLIGALAFGHLPAI |
| | | | FMPAGPMTPGIPNKQKAEVRER |
| | | | YAAGEATRAELLEAESSSYHSP |
| | | | GTCTFYGTANSNQVLLEAMGVQ |
| | | | LPGASFVNPELPLRDALTREGT |
| | | | ARALAISALGDDFRPFGRLIDE |
| | | | RAIVNAVVALMATGGSTNHTIH |
| | | | WIAVARAAGIVLTWDDMDLISQ |
| | | | TVPLLTRIYPNGEADVNRFQAA |
| | | | GGTAFVFRELMDAGYMHDDLPT |
| | | | IVEGGMRAYVNEPRLQDGKVTY |
| | | | VPGTATTADDSVARPVSDAFES |
| | | | QGGLRLLRGNLGRSLIKLSAVK |
| | | | PQHRSIQAPAVVIDTPQVLNKL |
| | | | HAAGVLPHDFVVVLRYQGPRAN |
| | | | GMPELHSMAPLLGLLQNQGRRV |
| | | | ALVTDGRLSGASGKFPAAIHMT |
| | | | PEAARGGPIGRVREGDIVRLDG |
| | | | EAGTLEVLVSAEEWASREVAPN |
| | | | TALAGNDLGRNLFAINRQVVGP |
| | | | ADQGAISISCGPTHPDGALWSY |
| | | | DAEYELGADAAAAAAPHESKDA |
| NP_791117.1 | Pseudomonas | Pv. | MHPRVLEVTERLIARSRDTRQR |
| | syringae | tomato | YLQLIRGAASDGPMRGKLQCAN |
| | | str. | FAHGVAACGPEDKQSLRLMNAA |
| | | DC3000 | NVAIVSSYNEMLSAHQPYEHFP |
| | | | AQIKQALRDIGSVGQFAGGVPA |
| | | | MCDGVTQGEPGMELAIASREVI |
| | | | AMSTAIALSHNMFDAAMMLGIC |
| | | | DKIVPGLMMGALRFGHLPTIFV |
| | | | PGGPMVSGISNKEKADVRQRYA |
| | | | EGKASREELLDSEMKSYHGPGT |
| | | | CTFYGTANTNQLVMEVMGMHLP |
| | | | GASFVNPYTPLRDALTAEAARQ |
| | | | VTRLTMQSGSFMPIGEIVDERS |
| | | | LVNSIVALHATGGSTNHTLHMP |
| | | | AIAQAAGIQLTWQDMADLSEVV |
| | | | PTLSHVYPNGKADINHFQAAGG |
| | | | MSFLIRELLAAGLLHENVNTVA |
| | | | GYGLSRYTKEPFLEDGKLVWRE |
| | | | GPLDSLDENILRPVARPFSPEG |
| | | | GLRVMEGNLGRGVMKVSAVALE |
| | | | HQIVEAPARVFQDQKELADAFK |
| | | | AGELECDFVAVMRFQGPRCNGM |
| | | | PELHKMTPFLGVLQDRGFKVAL |
| | | | VTDGRMSGASGKIPAAIHVCPE |
| | | | AFDGGPLALVRDGDVIRVDGVK |
| | | | GTLQVLVEASELAAREPAINQI |
| | | | DNSVGCGRELFGFMRMAFSSAE |
| | | | QGASAFTSSLETLK |
| YP_261706.1 | Pseudomonas | Pf-5 | MHPRVLEVTERLIARSRATRQA |
| | fluorescens | | YLALIRDAASDGPQRGKLQCAN |
| | | | FAHGVAGCGTDDKHNLRMMNAA |
| | | | NVAIVSSYNDMLSAHQPYEVFP |
| | | | EQIKRALREIGSVGQFAGGTPA |
| | | | MCDGVTQGEAGMELSLPSREVI |
| | | | ALSTAVALSHNMFDAALMLGIC |
| | | | DKIVPGLMMGALRFGHLPTIFV |
| | | | PGGPMVSGISNKQKADVRQRYA |
| | | | EGKASREELLESEMKSYHSPGT |
| | | | CTFYGTANTNQLLMEVMGLHLP |
| | | | GASFVNPNTPLRDALTHEAAQQ |
| | | | VTRLTKQSGAFMPIGEIVDERV |
| | | | LVNSIVALHATGGSTNHTLHMP |
| | | | AIAQAAGIQLTWQDMADLSEVV |
| | | | PTLSHVYPNGKADINHFQAAGG |
| | | | MSFLIRELLEAGLLHEDVNTVA |
| | | | GRGLSRYTQEPFLDNGKLVWRD |
| | | | GPIESLDENILRPVARAFSAEG |
| | | | GLRVMEGNLGRGVMKVSAVAPE |
| | | | HQIVEAPAVVFQDQQDLADAFK |
| | | | AGLLEKDFVAVMRFQGPRSNGM |
| | | | PELHKMTPFLGVLQDRGFKVAL |
| | | | VTDGRMSGASGKIPAAIHVSPE |
| | | | AQVGGALARVLDGDIIRVDGVK |
| | | | GTLELKVDAAEFAAREPAKGLL |
| | | | GNNVGTGRELFAFMRMAFSSAE |
| | | | QGASAFTSALETLK |
| ZP_0359148.1 | Bacillus | Subtilis | MAELRSNMITQGIDRAPHRSLL |
| | Subtilis | str. | RAAGVKEEDFGKPFIAVCNSYI |
| | | 168 | DIVPGHVHLQEFGKIVKEAIRE |
| | | | AGGVPFEFNTIGVDDGIAMGHI |
| | | | GMRYSLPSREIIADSVETVVSA |
| | | | HWFDGMVCIPNCDKITPGMLMA |
| | | | AMRINIPTIFVSGGPMAAGRTS |
| | | | YGRKISLSSVFEGVGAYQAGKI |
| | | | NENELQELEQFGCPTCGSCSGM |
| | | | FTANSMNCLSEALGLALPGNGT |
| | | | ILATSPERKEFVRKSAAQLMET |
| | | | IRKDIKPRDIVTVKAIDNAFAL |
| | | | DMALGGSTNTVLHTLALANEAG |
| | | | VEYSLERINEVAERVPHLAKLA |
| | | | PASDVFIEDLHEAGGVSAALNE |
| | | | LSKKEGALHLDALTVTGKTLGE |
| | | | TIAGHEVKDYDVIHPLDQPFTE |
| | | | KGGLAVLFGNLAPDGAIIKTGG |
| | | | VQNGITRHEGPAVVFDSQDEAL |
| | | | DGIINRKVKEGDVVIIRYEGPK |
| | | | GGPGMPEMLAPTSQIVGMGLGP |
| | | | KVALITDGRFSGASRGLSIGHV |
| | | | SPEAAEGGPLAFVENGDHIIVD |
| | | | IEKRILDVQVPEEEWEKRKANW |
| | | | KGFEPKVKTGYLARYSKLVTSA |
| | | | NTGGIMKI |
| YP_091897.1 | Bacillus | ATCC | MTGLRSDMITKGIDRAPHRSLL |
| | licheniformis | 14580 | RAAGVKEEDFGKPFIAVCNSYI |
| | | | DIVPGHVHLQEFGKIVKEAIRE |
| | | | AGGVPFEFNTIGVDDGIAMGHI |
| | | | GMRYSLPSREIIADSVETVVSA |
| | | | HWFDGMVCIPNCDKITPGMIMA |
| | | | AMRINIPTVFVSGGPMEAGRTS |
| | | | DGRKISLSSVFEGVGAYQSGKI |
| | | | DEKGLEELEQFGCPTCGSCSGM |
| | | | FTANSMNCLSEALGIAMPGNGT |
| | | | ILATSPDRREFAKQSARQLMEL |
| | | | IKSDIKPRDIVTEKAIDNAFAL |
| | | | DMALGGSTNTILHTLAIANEAG |
| | | | VDYSLERINEVAARVPHLSKLA |
| | | | PASDVFIEDLHEAGGVSAVLNE |
| | | | LSKKEGALHLDTLTVTGKTLGE |
| | | | NIAGREVKDYEVIHPIDQPFSE |
| | | | QGGLAVLFGNLAPDGAIIKTGG |
| | | | VQDGITRHEGPAVVFDSQEEAL |
| | | | DGIINRKVKAGDVVIIRYEGPK |
| | | | GGPGMPEMLAPTSQIVGMGLGP |
| | | | KVALITDGRFSGASRGLSIGHV |
| | | | SPEAAEGGPLAFVENGDHIVVD |
| | | | IEKRILNIEISDEEWEKRKANW |
| | | | PGFEPKVKTGYLARYSKLVTSA |
| | | | NTGGIMKI |
| NP_0718074.1 | Sewanella | MR-1 | MHSVVQSVTDRIIARSKASREA |
| | oneidensis | | YLAALNDARNHGVHRSSLSCGN |
| | | | LAHGFAACNPDDKNALRQLTKA |
| | | | NIGIITAFNDMLSAHQPYETYP |
| | | | DLLKKACQEVGSVAQVAGGVPA |
| | | | MCDGVTQGQPGMELSLLSREVI |
| | | | AMATAVGLSHNMFDGALLLGIC |
| | | | DKIVPGLLIGALSFGHLPMLFV |
| | | | PAGPMKSGIPNKEKARIRQQFA |
| | | | QGKVDRAQLLEAEAQSYHSAGT |
| | | | CTFYGTANSNQLMLEVMGLQLP |
| | | | GSSFVNPDDPLREALNKMAAKQ |
| | | | VCRLTELGTQYSPIGEVVNEKS |
| | | | IVNGIVALLATGGSTNLTMHIV |
| | | | AAARAAGIIVNWDDFSELSDAV |
| | | | PLLARVYPNGHADINHFHAAGG |
| | | | MAFLIKELLDAGLLHEDVNTVA |
| | | | GYGLRRYTQEPKLLDGELRWVD |
| | | | GPTVSLDTEVLTSVATPFQNNG |
| | | | GLKLLKGNLGRAVIKVSAVQPQ |
| | | | HRVVEAPAVVIDDQNKLDALFK |
| | | | SGALDRDCVVVVKGQGPKANGM |
| | | | PELHKLTPLLGSLQDKGFKVAL |
| | | | MTDGRMSGASGKVPAAIHLTPE |
| | | | AIDGGLIAKVQDGDLIRVDALT |
| | | | GELSLLVSDTELATRTATEIDL |
| | | | RHSRYGMGRELFGVLRSNLSSP |
| | | | ETGARSTSAIDELY |
| YP_190870.1 | Gluconobacter | 621H | MSLNPVVESVTARIIERSKVSR |
| | oxydans | | RRYLALMERNRAKGVLRPKLAC |
| | | | GNLAHAIAASSPDKPDLMRPTG |
| | | | TNIGVITTYNDMLSAHQPYGRY |
| | | | PEQIKLFAREVGATAQVAGGAP |
| | | | AMCDGVTQGQEGMELSLFSRDV |
| | | | IAMSTAVGLSHGMFEGVALLGI |
| | | | CDKIVPGLLMGALRFGHLPAML |
| | | | IPAGPMPSGLPNKEKQRIRQLY |
| | | | VQGKVGQDELMEAENASYHSPG |
| | | | TCTFYGTANTNQMMVEIMGLMM |
| | | | PDSAFINPNTKLRQAMTRSGIH |
| | | | RLAEIGLNGEDVRPLAHCVDEK |
| | | | AIVNAAVGLLATGGSTNHSIHL |
| | | | PAIARAAGILIDWEDISRLSSA |
| | | | VPLITRVYPSGSEDVNAFNRVG |
| | | | GMPTVIAELTRAGMLHKDILTV |
| | | | SRGGFSDYARRASLEGDEIVYT |
| | | | HAKPSTDTDILRDVATPFRPDG |
| | | | GMRLMTGNLGRAIYKSSAIAPE |
| | | | HLTVEAPARVFQDQHDVLTAYQ |
| | | | NGELERDVVVVVRFQGPEANGM |
| | | | PELHKLTPTLGVLQDRGFKVAL |
| | | | LTDGRMSGASGKVPAAIHVGPE |
| | | | AQVGGPIARVRDGDMIRVCAVT |
| | | | GQIEALVDAAEWESRKPVPPPL |
| | | | PALGTGRELFALMRSVHDPAEA |
| | | | GGSAMLAQMDRVIEAVGDDIH |
| ZP_06145432.1 | Ruminococcus | FD-1 | MSDNFFCEGADKAPQRSLFNAL |
| | flavefaciens | | GMTKEEMKRPLVGIVSSYNEIV |
| | | | PGHMNIDKLVEAVKLGVAMGGG |
| | | | TPVVFPAIAVCDGIAMGHTGMK |
| | | | YSLVTRDLIADSTECMALAHHF |
| | | | DALVMIPNCDKNVPGLLMAAAR |
| | | | INVPTVFVSGGPMLAGHVKGKK |
| | | | TSLSSMFEAVGAYTAGKIDEAE |
| | | | LDEFENKTCPTCGSCSGMYTAN |
| | | | SMNCLTEVLGMGLRGNGTIPAV |
| | | | YSERIKLAKQAGMQVMELYRKN |
| | | | IRPLDIMTEKAFQNALTADMAL |
| | | | GCSTNSMLHLPAIANECGININ |
| | | | LDMANEISAKTPNLCHLAPAGH |
| | | | TYMEDLNEAGGVYAVLNELSKK |
| | | | GLINTDCMTVTGKTVGENIKGC |
| | | | INRDPETIRPIDNPYSETGGIA |
| | | | VLKGNLAPDRCVVKRSAVAPEM |
| | | | LVHKGPARVFDSEEEAIKVIYE |
| | | | GGIKAGDVVVIRYEGPAGGPGM |
| | | | REMLSPTSAIQGAGLGSTVALI |
| | | | TDGRFSGATRGAAIGHVSPEAV |
| | | | NGGTIAYVKDGDIISIDIPNYS |
| | | | ITLEVSDEELAERKKAMPIKRK |
| | | | ENITGYLKRYAQQVSSADKGAI |
| | | | INRK |

### Example 10

### Testing of various EDA sources, independently of EDD genes, to identify the best EDA candidate.

Various EDA genes were tested, independently of EDD, in order to determine suitable EDA genes for expression in S. cerevisiae (e.g., for the C6 strain). The EDA activity was independently assessed by adding saturating amounts of over expressed E. coli EDD extracts to S. cerevisiae EDA extracts lacking EDD (Cheriyan et al., Protein Science 16:2368-2377, 2007). The relative activities of EDA's in S. cerevisiae were also ranked this way. The activity of integrated EDA's in Thermosacc-Gold haploids were also analyzed in this manner. The table below describes the primers used to isolate the additional EDA genes.

### Cloning of New EDA Sources

| # | Name | Description | Sequence |
|---|---|---|---|
| 726 | KA/EDA- | Cloning primer for Shewanella | GTTCACTGC |
| | SoFor | oneidensis EDA | ACTAGTAAAAAAATGCTTGAGAAT |
| | | | AACTGGTC |
| 727 | KA/EDA- | Cloning primer for Shewanella | CTTCGAGATCTCGAGTTAAAGTCC |
| | SoRev | oneidensis EDA | GCCAATCGCCTC |
| 728 | KA/EDA- | Cloning primer for | GTTCACTGCACTAGTAAAAAAATG |
| | GoFor | Gluconobacter oxydans EDA | ATCGATACTGCCAAACTC |
| 729 | KA/EDA- | Cloning primer for | CTTCGAGATCTCGAGTCAGACCG |
| | GoRev | Gluconobacter oxydans EDA | TGAAGAGTGCCGC |
| 837 | KA/EDA- | Cloning primer for Bacillus | GTTCACTGCACTAGTAAAAAAATG |
| | BLFor | licheniformis EDA | GTATTGTCACACATCGAAG |
| 838 | KA/EDA- | Cloning primer for Bacillus | CTTCGAGATCTCGAGTTACTGTTT |
| | BLRev | licheniformis EDA | TGCTGCTTCAACAAATTG |
| 839 | KA/EDA- | Cloning primer for Bacillus | GTTCACTGCACTAGTAAAAAAATG |
| | BsFor | subtilis EDA | GAGTCCAAAGTCGTTGAAAACC |
| 840 | KA/EDA- | Cloning primer for Bacillus | CTTCGAGATCTCGAGTTACACTTG |
| | BsRev | subtilis EDA | GAAAACAGCCTGCAAATCC |
| 841 | KA/EDA- | Cloning primer for | GTTCACTGCACTAGTAAAAAAATG |
| | PfFor | Pseudomonas fluorescens | ACAAACCTCGCCCCGACC |
| | | EDA | |
| 842 | KA/EDA- | Cloning primer for | CTTCGAGATCTCGAGTCAGTCCA |
| | PfRev | Pseudomonas fluorescens | GCAGGGCCAGG |
| | | EDA | |
| 843 | KA/EDA- | Cloning primer for | GTTCACTGCACTAGTAAAAAAATG |
| | PsFor | Pseudomonas syringae EDA | ACACAGAACGAAAATAATCAGCCGC |
| 844 | KA/EDA- | Cloning primer for | CTTCGAGATCTCGAGTCAGTCAAA |
| | PsRev | Pseudomonas syringae EDA | CAGCGCCAGCGC |
| 845 | KA/EDA- | Cloning primer for | GTTCACTGCACTAGTAAAAAAATG |
| | SdFor | Saccharophagus degradans | GCTATTACAAAAGAATTTTTAGCT |
| | | EDA | CCAG |
| 846 | KA/EDA- | Cloning primer for | CTTCGAGATCTCGAGTTAGCTAGA |
| | SdRev | Saccharophagus degradans | AATTTTAGCGGTAGTTGCC |
| | | EDA | |
| 847 | KA/EDA- | Cloning primer for | GTTCACTGCACTAGTAAAAAAATG |
| | XaFor | Xanthomonas axonopodis | ACGATTGCCCAGACCCAG |
| | | EDA | |
| 848 | KA/EDA- | Cloning primer for | CTTCGAGATCTCGAGTCAGCCCG |
| | XaRev | Xanthomonas axonopodis | CCCGCACC |
| | | EDA | |
| 835 | KA/NdeI | Cloning primer for E. coli EDD | GTTCACTGCCATATGAATCCACAA |
| | EDDfor | | TTGTTACGCGTAACAAATCGAATC |
| | | | ATTG |
| 836 | KA/XhoI | Cloning primer for E. coli EDD | CTTCGAGATCTCGAGTTAAAAAGT |
| | EDDrev | | GATACAGGTTGCGCCCTGTTCGGC |

Listed in the table below are EDA sequences and Accession numbers for the isolated alternate EDA genes.

| Accession | | Strain | |
|---|---|---|---|
| Number | Species | Number | Nucleotide Sequence |
| YP_526856.1 | Saccharophagus | 2-40 | atggctattacaaaagaatttttagctccagttggcgtaatgcctg |
| | degradans | | ttgtggttgtggatcgtgtagaagatgcggtgcctattacaaacgc |
| | | | attaaaagccggcggtattaaagcagttgagattactttacgtact |
| | | | cctgcggcactggatgctattcgcgctattaaagctgagtgtgaag |
| | | | acatcctggtgggggtaggtacggttattaaccatcaaaaccttaa |
| | | | agatattgctgcaattggtgttgatttcgccgtatctcctggttac |
| | | | accccaacattgctgaagcaagcgcaagatttgggcgtagaaatgt |
| | | | tgcctggtgtaacttcgccttctgaagttatgcttggtatggagct |
| | | | aggtttgtcttgcttcaagctattccctgcggttgcagtaggtggt |
| | | | ttgccattacttaagtctattggtggcccattaccacaggtttcct |
| | | | tctgtccaacaggcggtttgactatcgatactttcaccgacttctt |
| | | | ggcattgcctaacgttgcttgtgtgggtggtacttggttggtgcct |
| | | | gcagatgctgttgcagctaaaaactggcaagctattactgatattg |
| | | | cggcggcaactaccgctaaaatttctagctaa |
| | Xanthomonas | ATCC | atgacgattgcccagacccagaacaccgccgaacagttgctgcgcg |
| | axonopodis | 13902 | atgccggcatcttgcccgtggtcaccgtggacacgctggatcaggc |
| | pv. | | gcgccgcgtcgccgatgcgttgctcgaaggcggcctgcccgcgatc |
| | Vasculorum | | gagctgacccttcgcacgccagtggcgatcgacgcgctggcgatgc |
| | | | tcaagcgcgagcttcctaacatcttgatcggtgccggcaccgtgct |
| | | | gagcgaattgcagctgcgtcagtcggtggatgccggtgcagacttc |
| | | | ctggtgaccccgggcacgccggcgccgctggcgcgcctgctggcgg |
| | | | atgcgccgatcccggccgttcccggcgcggccactccgaccgagct |
| | | | gctgaccttgatgggtcttggctttcgcgtctgcaagctgttcccg |
| | | | gccaccgccgtgggcggtctgcagatgctcaggggcctggccggcc |
| | | | cgctgtccgagctcaagctgtgccccaccggcggcatcagcgaggc |
| | | | caacgccgccgagttcctgtcgcagccgaacgtgctgtgcatcggc |
| | | | ggttcgtggatggtccccaaggattggctggcgcacggccaatggg |
| | | | acaaggtcaaggaaagctcggccaaggcggcggcgatcgtgcggca |
| | | | ggtgcgggcgggctga |
| AAO55695.1 | Pseudomonas | Pv. | atgacacagaacgaaaataatcagccgctcaccagcatggcgaaca |
| | syringiae | Tomato | agattgcccggatcgacgaactctgcgccaaggcaaagattctgcc |
| | | str | ggtcatcaccattgcccgtgatcaggacgtattgccactggccgac |
| | | DC3000 | gcgctggccgctggtggcatgacggctctggaaatcaccctgcgct |
| | | | cggcgttcggactgagtgcgatccgcattttgcgcgagcagcgccc |
| | | | agagctgtgcactggcgccgggaccattctggaccgcaagatgctg |
| | | | gccgacgccgaggcggcgggctcgcaattcattgtgacccccggca |
| | | | gcacgcaggaactgttgcaggcggcgctcgacagcccgttgcccct |
| | | | gttgccaggcgtcagcagcgcgtcggaaatcatgatcggctatgcc |
| | | | ttgggttatcgccgcttcaagctgttcccggcagaaatcagcggcg |
| | | | gtgtggcagcgatcaaggccttgggcgggcctttcaacgaggtgcg |
| | | | tttctgcccgacgggcggcgtcaacgagcagaacctcaagaactac |
| | | | atggccttgcccaacgtcatgtgcgtcggcgggacatggatgattg |
| | | | ataacgcctgggtcaagaatggcgactggggccgcattcaggaagc |
| | | | cacggcacaggcgctggcgctgtttgactga |
| NP_718073.1 | Shewanella | MR-1 | atgcttgagaataactggtcattacaaccacaagatatttttaaac |
| | oneidensis | | gcagccctattgttcctgttatggtgattaacaagattgaacatgc |
| | | | ggtgcccttagctaaagcgctggttgccggagggataagcgtgttg |
| | | | gaagtgacattacgcacgccatgcgcccttgaagctatcaccaaaa |
| | | | tcgccaaggaagtgcctgaggcgctggttggcgcggggactatttt |
| | | | aaatgaagcccagcttggacaggctatcgccgctggtgcgcaattt |
| | | | attatcactccaggtgcgacagttgagctgctcaaagcgggcatgc |
| | | | aaggaccggtgccgttaattccgggcgttgccagtatttccgaggt |
| | | | gatgacgggcatggcgctgggctacactcactttaaattcttccct |
| | | | gctgaagcgtcaggtggcgttgatgcgcttaaggctttctctgggc |
| | | | cgttagcagatatccgcttctgcccaacaggtggaattaccccgag |
| | | | cagctataaagattacttagcgctgaagaatgtcgattgtattggt |
| | | | ggcagctggattgctcctaccgatgcgatggagcagggcgattggg |
| | | | atcgtatcactcagctgtgtaaagaggcgattggcggactttaa |
| YP_261692 | Pseudomonas | Pf-5 | atgacaaacctcgccccgaccgtttccatggcggacaaagttgccc |
| | fluorescens | | tgatcgacagcctctgcgccaaggcgcggatcctgccggtgatcac |
| | | | cattgcccgcgagcaggatgtcctgccgctggccgatgccctggcg |
| | | | gccggcggcctgaccgccctggaagtgaccctgcgttcgcagttcg |
| | | | gcctcaaggcgatccagatcctgcgcgaacagcgcccggagctggt |
| | | | gaccggtgccggcaccgtgctcgacccgcagatgctggtggcggcg |
| | | | gaagcggcaggttcgcagttcatcgtcaccccgggcatcacccgcg |
| | | | acctgctgcaagccagcgtggccagcccgattcccctgctgccggg |
| | | | gatcagcaatgcctccgggatcatggagggttatgccctgggctac |
| | | | cgccgcttcaagctgttcccggcggaagtcagtggtggcgtggcgg |
| | | | cgatcaaggccctgggcgggccgttcggcgaggtcaagttctgccc |
| | | | taccggcggcgtcggcccggccaatatcaagagctacatggcgctc |
| | | | aagaatgtgatgtgtgtcggcggtagctggatgctcgatcccgagt |
| | | | ggatcaagaacggcgactgggcacggatccaggagtgcacggccga |
| | | | ggccctggccctgctggactga |
| ZP_03591973.1 | Bacillus | subtilis | atggagtccaaagtcgttgaaaaccgtctgaaagaagcaaagctga |
| | subtilis | str. 168 | ttgcagtcattcgttcaaaggataagcaggaggcctgtcagcagat |
| | | | tgagagtttattagataaagggattcgtgcagttgaagtgacgtat |
| | | | acgacccccggggcatcagatattatcgaatccttccgtaataggg |
| | | | aagatattttaattggcgcgggtacggtcatcagcgcgcagcaagc |
| | | | tggggaagctgctaaggctggcgcgcagtttattgtcagtccgggt |
| | | | ttttcagctgatcttgctgaacatctatcttttgtaaagacacatt |
| | | | atatccccggcgtcttgactccgagcgaaattatggaagcgctgac |
| | | | attcggttttacgacattaaagctgttcccaagcggtgtgtttggc |
| | | | attccgtttatgaaaaatttagcgggtcctttcccgcaggtgacct |
| | | | ttattccgacaggcgggatacatccgtctgaagtgcctgattggct |
| | | | tagagccggagctggcgccgtcggagtcggcagccagttgggcagc |
| | | | tgttcaaaagaggatttgcaggctgttttccaagtgtaa |
| YP_081150.2 | Bacillus | ATCC | atggtattgtcacacatcgaagaacaaaaactgattgcgatcatcc |
| | licheniformis | 14580 | gcggatacaatccggaggaggcagtgagcattgccggcgccttaaa |
| | | | agcgggcggcatcaggcttgtggagattacgcttaattcccctcaa |
| | | | gcgatcaaagcgattgaagcggtttcagagcattttggggacgaaa |
| | | | tgcttgtcggagcgggaaccgtacttgatcccgaatctgcgagagc |
| | | | ggcgcttttagccggcgcgcggtttatcctgtctccgaccgtcaat |
| | | | gaagagacgatcaagctgacaaaacggtatggagcggtcagcattc |
| | | | caggcgcttttaccccgactgaaatattgacggcgtatgaaagcgg |
| | | | gggagacatcatcaaggtatttcccggaacaatggggcctggctat |
| | | | atcaaggatatccacggaccgcttccgcatattccgctgcttccga |
| | | | ctggaggagtcggattggaaaaccttcacgagtttctgcaggccgg |
| | | | tgcggtcggagcgggaatcggcggttcgcttgttcgggctaataaa |
| | | | gatgttaatgacgcgtttttagaagagctgtccaaaaaagcaaagc |
| | | | aatttgttgaagcagcaaaacagtaa |
| YP_190869.1 | Gluconobacter | 62IH | atgatcgatactgccaaactcgacgccgtcatgagccgttgtccgg |
| | oxydans | | tcatgccggtgctggtggtcaatgatgtggctctggcccgcccgat |
| | | | ggccgaggctctggtggcgggtggactgtccacgctggaagtcacg |
| | | | ctgcgcacgccctgcgcccttgaagctattgaggaaatgtcgaaag |
| | | | taccaggcgcgctggtcggtgccggtacggtgctgaatccgtccga |
| | | | catggaccgtgccgtgaaggcgggtgcgcgcttcatcgtcagcccc |
| | | | ggcctgaccgaggcgctggcaaaggcgtcggttgagcatgacgtcc |
| | | | ccttcctgccaggcgttgccaatgcgggtgacatcatgcggggtct |
| | | | ggatctgggtctgtcacgcttcaagttcttcccggctgtgacgaat |
| | | | ggcggcattcccgcgctcaagagcttggccagtgtttttggcagca |
| | | | atgtccgtttctgccccacgggcggcattacggaagagagcgcacc |
| | | | ggactggctggcgcttccctccgtggcctgcgtcggcggatcctgg |
| | | | gtgacggccggcacgttcgatgcggacaaggtccgtcagcgcgcca |
| | | | cggctgcggcactcttcacggtctga |
| NP_251871.1 | P. aeruginosa | PAO1 | Atgaaaaactggaaaacaagtgcagaatcaatcctgaccaccggcc |
| | | Codon | cggttgtaccggttatcgtggtaaaaaaactggaacacgcggtgcc |
| | | Optimized | gatggcaaaagcgttggttgctggtggggtgcgcgttctggaagtg |
| | | | actctgcgtaccgagtgtgcagttgacgctatccgtgctatcgcca |
| | | | aagaagtgcctgaagcgattgtgggtgccggtacggtgctgaatcc |
| | | | acagcagctggcagaagtcactgaagcgggtgcacagttcgcaatt |
| | | | agcccgggtctgaccgagccgctgctgaaagctgctaccgaaggga |
| | | | ctattcctctgattccggggatcagcactgtttccgaactgatgct |
| | | | gggtatggactacggtttgaaagagttcaaattcttcccggctgaa |
| | | | gctaacggcggcgtgaaagccctgcaggcgatcgcgggtccgttct |
| | | | cccaggtccgtttctgcccgacgggtggtatttctccggctaacta |
| | | | ccgtgactacctggcgctgaaaagcgtgctgtgcatcggtggttcc |
| | | | tggctggttccggcagatgcgctggaagcgggcgattacgaccgca |
| | | | ttactaagctggcgcgtgaagctgtagaaggcgctaagctgtaa |
| | PAO1-Ec5 | | atgaaaaactggaaacagaagaccgcccgcatcgacacgctgtgcc |
| | | | gggaggcgcgcatcctcccggtgatcaccatcgaccgcgaggcgga |
| | | | catcctgccgatggccgatgccctcgccgccggcggcctgaccgcc |
| | | | ctggagatcaccctgcgcacggcgcacgggctgaccgccatccggc |
| | | | gcctcagcgaggagcgcccgcacctgcgcatcggcgccggcaccgt |
| | | | gctcgacccgcggaccttcgccgccgcggaaaaggccggggcgagc |
| | | | ttcgtggtcaccccgggttgcaccgacgagttgctgcgcttcgccc |
| | | | tggacagcgaagtcccgctgttgcccggcgtggccagcgcttccga |
| | | | gatcatgctcgcctaccgccatggctaccgccgcttcaagctgttt |
| | | | cccgccgaagtcagcggcggcccggcggcgctgaaggcgttctcgg |
| | | | gaccattccccgatatccgcttctgccccaccggaggcgtcagcct |
| | | | gaacaatctcgccgactacctggcggtacccaacgtgatgtgcgtc |
| | | | ggcggcacctggatgctgcccaaggccgtggtcgaccgcggcgact |
| | | | gggcccaggtcgagcgcctcagccgcgaagccctggagcgcttcgc |
| | | | cgagcaccgcagacactaatagctcgagttactttact |
| | PAO1- | | atgaaaaactggaaaacaagtgcagaatcaatcgacacgctgtgcc |
| | Ec10 | | gggaggcgcgcatcctcccggtgatcaccatcgaccgcgaggcgga |
| | | | catcctgccgatggccgatgccctcgccgccggcggcctgaccgcc |
| | | | ctggagatcaccctgcgcacggcgcacgggctgaccgccatccggc |
| | | | gcctcagcgaggagcgcccgcacctgcgcatcggcgccggcaccgt |
| | | | gctcgacccgcggaccttcgccgccgcggaaaaggccggggcgagc |
| | | | ttcgtggtcaccccgggttgcaccgacgagttgctgcgcttcgccc |
| | | | tggacagcgaagtcccgctgttgcccggcgtggccagcgcttccga |
| | | | gatcatgctcgcctaccgccatggctaccgccgcttcaagctgttt |
| | | | cccgccgaagtcagcggcggcccggcggcgctgaaggcgttctcgg |
| | | | gaccattccccgatatccgcttctgccccaccggaggcgtcagcct |
| | | | gaacaatctcgccgactacctggcggtacccaacgtgatgtgcgtc |
| | | | ggcggcacctggatgctgcccaaggccgtggtcgaccgcggcgact |
| | | | gggcccaggtcgagcgcctcagccgcgaagccctggagcgcttcgc |
| | | | cgagcaccgcagacactaatagctcgagttactttact |
| | PAO1- | | atgaaaaactggaaaacaagtgcagaatcaatcctgaccaccggcc |
| | Ec15 | | gggaggcgcgcatcctcccggtgatcaccatcgaccgcgaggcgga |
| | | | catcctgccgatggccgatgccctcgccgccggcggcctgaccgcc |
| | | | ctggagatcaccctgcgcacggcgcacgggctgaccgccatccggc |
| | | | gcctcagcgaggagcgcccgcacctgcgcatcggcgccggcaccgt |
| | | | gctcgacccgcggaccttcgccgccgcggaaaaggccggggcgagc |
| | | | ttcgtggtcaccccgggttgcaccgacgagttgctgcgcttcgccc |
| | | | tggacagcgaagtcccgctgttgcccggcgtggccagcgcttccga |
| | | | gatcatgctcgcctaccgccatggctaccgccgcttcaagctgttt |
| | | | cccgccgaagtcagcggcggcccggcggcgctgaaggcgttctcgg |
| | | | gaccattccccgatatccgcttctgccccaccggaggcgtcagcct |
| | | | gaacaatctcgccgactacctggcggtacccaacgtgatgtgcgtc |
| | | | ggcggcacctggatgctgcccaaggccgtggtcgaccgcggcgact |
| | | | gggcccaggtcgagcgcctcagccgcgaagccctggagcgcttcgc |
| | | | cgagcaccgcagacactaatagctcgagttactttact |

| Accession | | Strain | |
|---|---|---|---|
| Number | Species | Number | Amino Acid Sequence |
| YP_526856.1 | Saccharophagus | 2-40 | MAITKEFLAPVGVMPVVVVDRV |
| | degradans | | EDAVPITNALKAGGIKAVEITL |
| | | | RTPAALDAIRAIKAECEDILVG |
| | | | VGTVINHQNLKDIAAIGVDFAV |
| | | | SPGYTPTLLKQAQDLGVEMLPG |
| | | | VTSPSEVMLGMELGLSCFKLFP |
| | | | AVAVGGLPLLKSIGGPLPQVSF |
| | | | CPTGGLTIDTFTDFLALPNVAC |
| | | | VGGTWLVPADAVAAKNWQAITD |
| | | | IAAATTAKISS |
| | Xanthomonas | ATCC | MTIAQTQNTAEQLLRDAGILPV |
| | axonopodis | 13902 | VTVDTLDQARRVADALLEGGLP |
| | pv. | | AIELTLRTPVAIDALAMLKREL |
| | Vasculorum | | PNILIGAGTVLSELQLRQSVDA |
| | | | GADFLVTPGTPAPLARLLADAP |
| | | | IPAVPGAATPTELLTLMGLGFR |
| | | | VCKLFPATAVGGLQMLRGLAGP |
| | | | LSELKLCPTGGISEANAAEFLS |
| | | | QPNVLCIGGSWMVPKDWLAHGQ |
| | | | WDKVKESSAKAAAIVRQVRAG |
| AAO55695.1 | Pseudomonas | Pv. | MTQNENNQPLTSMANKIARIDE |
| | syringiae | Tomato | LCAKAKILPVITIARDQDVLPL |
| | | str | ADALAAGGMTALEITLRSAFGL |
| | | DC3000 | SAIRILREQRPELCTGAGTILD |
| | | | RKMLADAEAAGSQFIVTPGSTQ |
| | | | ELLQAALDSPLPLLPGVSSASE |
| | | | IMIGYALGYRRFKLFPAEISGG |
| | | | VAAIKALGGPFNEVRFCPTGGV |
| | | | NEQNLKNYMALPNVMCVGGTWM |
| | | | IDNAWVKNGDWGRIQEATAQAL |
| | | | ALFD |
| NP_718073.1 | Shewanella | MR-1 | MLENNWSLQPQDIFKRSPIVPV |
| | oneidensis | | MVINKIEHAVPLAKALVAGGIS |
| | | | VLEVTLRTPCALEAITKIAKEV |
| | | | PEALVGAGTILNEAQLGQAIAA |
| | | | GAQFIITPGATVELLKAGMQGP |
| | | | VPLIPGVASISEVMTGMALGYT |
| | | | HFKFFPAEASGGVDALKAFSGP |
| | | | LADIRFCPTGGITPSSYKDYLA |
| | | | LKNVDCIGGSWIAPTDAMEQGD |
| | | | WDRITQLCKEAIGGL |
| YP_261692 | Pseudomonas | Pf-5 | MTNLAPTVSMADKVALIDSLCA |
| | fluorescens | | KARILPVITIAREQDVLPLADA |
| | | | LAAGGLTALEVTLRSQFGLKAI |
| | | | QILREQRPELVTGAGTVLDPQM |
| | | | LVAAEAAGSQFIVTPGITRDLL |
| | | | QASVASPIPLLPGISNASGIME |
| | | | GYALGYRRFKLFPAEVSGGVAA |
| | | | IKALGGPFGEVKFCPTGGVGPA |
| | | | NIKSYMALKNVMCVGGSWMLDP |
| | | | EWIKNGDWARIQECTAEALALLD |
| ZP_03591973.1 | Bacillus | subtilis | MESKVVENRLKEAKLIAVIRSK |
| | subtilis | str. 168 | DKQEACQQIESLLDKGIRAVEV |
| | | | TYTTPGASDIIESFRNREDILI |
| | | | GAGTVISAQQAGEAAKAGAQFI |
| | | | VSPGFSADLAEHLSFVKTHYIP |
| | | | GVLTPSEIMEALTFGFTTLKLF |
| | | | PSGVFGIPFMKNLAGPFPQVTF |
| | | | IPTGGIHPSEVPDWLRAGAGAV |
| | | | GVGSQLGSCSKEDLQAVFQV |
| YP_081150.2 | Bacillus | ATCC | MVLSHIEEQKLIAIIRGYNPEE |
| | licheniformis | 14580 | AVSIAGALKAGGIRLVEITLNS |
| | | | PQAIKAIEAVSEHFGDEMLVGA |
| | | | GTVLDPESARAALLAGARFILS |
| | | | PTVNEETIKLTKRYGAVSIPGA |
| | | | FTPTEILTAYESGGDIIKVFPG |
| | | | TMGPGYIKDIHGPLPHIPLLPT |
| | | | GGVGLENLHEFLQAGAVGAGIG |
| | | | GSLVRANKDVNDAFLEELSKKA |
| | | | KQFVEAAKQ |
| YP_190869.1 | Gluconobacter | 62IH | MIDTAKLDAVMSRCPVMPVLVV |
| | oxydans | | NDVALARPMAEALVAGGLSTLE |
| | | | VTLRTPCALEAIEEMSKVPGAL |
| | | | VGAGTVLNPSDMDRAVKAGARF |
| | | | IVSPGLTEALAKASVEHDVPFL |
| | | | PGVANAGDIMRGLDLGLSRFKF |
| | | | FPAVTNGGIPALKSLASVFGSN |
| | | | VRFCPTGGITEESAPDWLALPS |
| | | | VACVGGSWVTAGTFDADKVRQR |
| | | | ATAAALFTV |
| NP_251871.1 | P. aeruginosa | PAO1 | MKNWKTSAESILTTGPVVPVIV |
| | | Codon | VKKLEHAVPMAKALVAGGVRVL |
| | | Optimized | EVTLRTECAVDAIRAIAKEVPE |
| | | | AIVGAGTVLNPQQLAEVTEAGA |
| | | | QFAISPGLTEPLLKAATEGTIP |
| | | | LIPGISTVSELMLGMDYGLKEF |
| | | | KFFPAEANGGVKALQAIAGPFS |
| | | | QVRFCPTGGISPANYRDYLALK |
| | | | SVLCIGGSWLVPADALEAGDYD |
| | | | RITKLAREAVEGAKL |
| | PAO1-Ec5 | | MKNWKQKTARIDTLCREARILP |
| | | | VITIDREADILPMADALAAGGL |
| | | | TALEITLRTAHGLTAIRRLSEE |
| | | | RPHLRIGAGTVLDPRTFAAAEK |
| | | | AGASFVVTPGCTDELLRFALDS |
| | | | EVPLLPGVASASEIMLAYRHGY |
| | | | RRFKLFPAEVSGGPAALKAFSG |
| | | | PFPDIRFCPTGGVSLNNLADYL |
| | | | AVPNVMCVGGTWMLPKAVVDRG |
| | | | DWAQVERLSREALERFAEHRRH |
| | PAO1- | | MKNWKTSAESIDTLCREARILP |
| | Ec10 | | VITIDREADILPMADALAAGGL |
| | | | TALEITLRTAHGLTAIRRLSEE |
| | | | RPHLRIGAGTVLDPRTFAAAEK |
| | | | AGASFVVTPGCTDELLRFALDS |
| | | | EVPLLPGVASASEIMLAYRHGY |
| | | | RRFKLFPAEVSGGPAALKAFSG |
| | | | PFPDIRFCPTGGVSLNNLADYL |
| | | | AVPNVMCVGGTWMLPKAVVDRG |
| | | | DWAQVERLSREALERFAEHRRH |
| | PAO1- | | MKNWKTSAESILTTGREARILP |
| | Ec15 | | VITIDREADILPMADALAAGGL |
| | | | TALEITLRTAHGLTAIRRLSEE |
| | | | RPHLRIGAGTVLDPRTFAAAEK |
| | | | AGASFVVTPGCTDELLRFALDS |
| | | | EVPLLPGVASASEIMLAYRHGY |
| | | | RRFKLFPAEVSGGPAALKAFSG |
| | | | PFPDIRFCPTGGVSLNNLADYL |
| | | | AVPNVMCVGGTWMLPKAVVDRG |
| | | | DWAQVERLSREALERFAEHRRH |

EDA and EDD extracts were prepared using the following protocol.

### Day 1

Grow 5 ml LB-Kan preps of BF1055 (BL21/DE3 with pET26b empty vector) and BF1706 (BL21 DE3 with pET26b+ E. coli EDD).

Grow 5 ml preps of each EDA construct expressed in S. cerevisiae in appropriate selective media (e.g. ScD-leu).

### Day 2

Grow 50 ml LB-Kan prep of BF1055, 2% (v/v) inoculate.

Grow 50 ml prep of BF1706 using Novagen's Overnight Express (46.45 ml LB-Kan, 1 ml solution 1, 2.5 ml solution 2, 50 μl solution 3, 5 μl of 1M MnCl₂, 50 μl of 0.5 M FeCl₂), 2% (v/v) inoculate.

Grow 50 ml prep of each EDA construct expressed in S. cerevisiae in appropriate selective media+10 mM MnCl₂. Inoculate to OD₆₀₀ of 0.2.

### Day 3

EDD extractions (adapted from Cheriyan et al, Protein Science 16:2368-2377, 2007):
1) Pellet cells in 50 ml conical tubes, 4° C., 3,000 rpm, 10 minutes, discard supernatant.
2) Resuspend in 2 ml degassed PDGH buffer (20 mM MES pH 6.5, 30 mM NaCl, 5 mM MnCl₂, 0.5 mM FeCl₂, 10 mM 2-mercaptoethanol, 10 mM cysteine, sparged with nitrogen gas). Move to hungate tube.
3) Add 0.1% Triton X-100, 10 ng/ml DNase, 10 μg/ml PMSF, 10 μg/ml TAME (Nα-(p-toluene sulfonyl)-L-arginine methyl ester), 100 μg/ml lysozyme.
4) Sparge hungate tube with nitrogen gas, cap and seal. Incubate 2 hours at 37° C., swirl occasionally.
5) Clarify by centrifugation in 2-ml tube, 4° C., 10 minutes, 14,000 rpm. Keep supernatant.
6) Treat with 150 mM pyruvate and 10 mM sodium cyanoborohydride (work in hood) to inactivate aldolase activity. Incubate 30 minutes at room temperature.
7) During incubation, pre-equilibrate PD-10 column from GE
   a. Remove top cap, pour off storage buffer.
   b. Cut off bottom tip, fit in 50 ml conical with adapter.
   c. Pour 5 ml of 20 mM MES buffer, pH 6.5 (total of 5 times). Discard flow-through.
8) Run sample through column, then add MES buffer to a total of 2.5 ml volume added. Discard flow-through.
9) Run 3.5 ml 20 mM MES pH 6.5 buffer to elute protein. Discard column in appropriate waste receptacle.
10) Perform Bradford assay (1:10 or 1:20 dilution).

### EDA Extractions:

1) Spin down in 50 ml conical tubes, 4° C., 3,400 rpm, 5 minutes. Wash 2× with 25 ml water.
2) Resuspend in 1 ml lysis buffer (50 mM Tris-HCl, pH 7, 10 mM MgCl₂, 1× protease inhibitor.
3) Add 1 cap of zirconia beads, vortex 4-6 times, 15 sec bursts, ice in between.
4) Spin down cell debris, 4° C., 14,000 rpm, 10 minutes. Save supernatant.
5) Perform Bradford assay (1:2 dilution).

### Activity Assays:

Each reaction contained 50 mM Tris-HCl, pH 7, 10 mM MgCl₂, 0.15 mM NADH, 15 μg LDH, saturating amounts of EDD determined empirically (usually ˜100 μg), 1-50 μg EDA (depending on level of activity), and 1 mM 6-phosphogluconate. Reactions were started by the addition of 6-phosphogluconate and monitored for 5 minutes at 30° C.

### Results

The S. cerevisiae strains tested for EDA activity are described in the table below. yCH strains are Thermosacc-based (Lallemand). BF strains are based on BY4742.

| Strain | Vector | Construct |
|---|---|---|
| BF542 | pBF150 | Zymomonas mobilis EDA |
| BF1689 | pBF892 | PAO1 + 5aa E. coli EDA |
| BF1691 | pBF894 | PAO1 + 10aa E. coli EDA |
| BF1693 | pBF896 | PAO1 + 15aa E. coli EDA |
| BF1721 | pBF909 | Bacillus licheniformis EDA |
| BF1722 | pBF910 | Bacillus subtilis EDA |
| BF1723 | pBF911 | Pseudomonas fluorescens EDA |
| BF1724 | pBF912 | Pseudomonas syringae EDA |
| BF1725 | pBF913 | Saccharophagus degradans EDA |
| BF1726 | pBF914 | Xanthomonas axonopodis EDA |
| BF1727 | pBF766 | Escherichia coli EDA |
| BF1728 | pBF764 | Pseudomonas aeruginosa EDA |
| BF1729 | pBF729 | Gluconobacter oxydans EDA |
| BF1730 | pBF727 | Shewanella oneidensis EDA |
| BF1775 | pBF87 | p425GPD (empty vector) |
| BF1776 | pBF928 | PAO1 EDA codon optimized for S. cerevisiae |

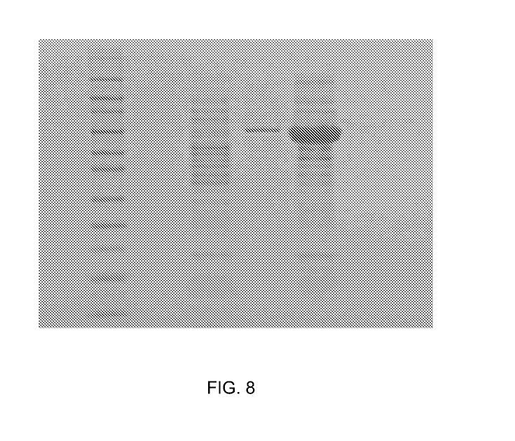

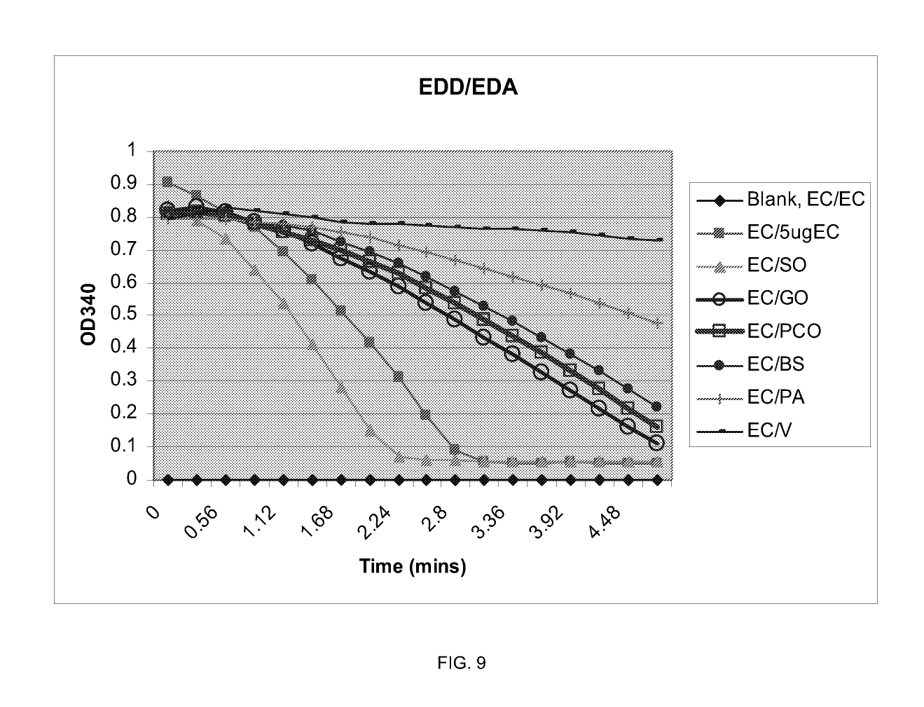

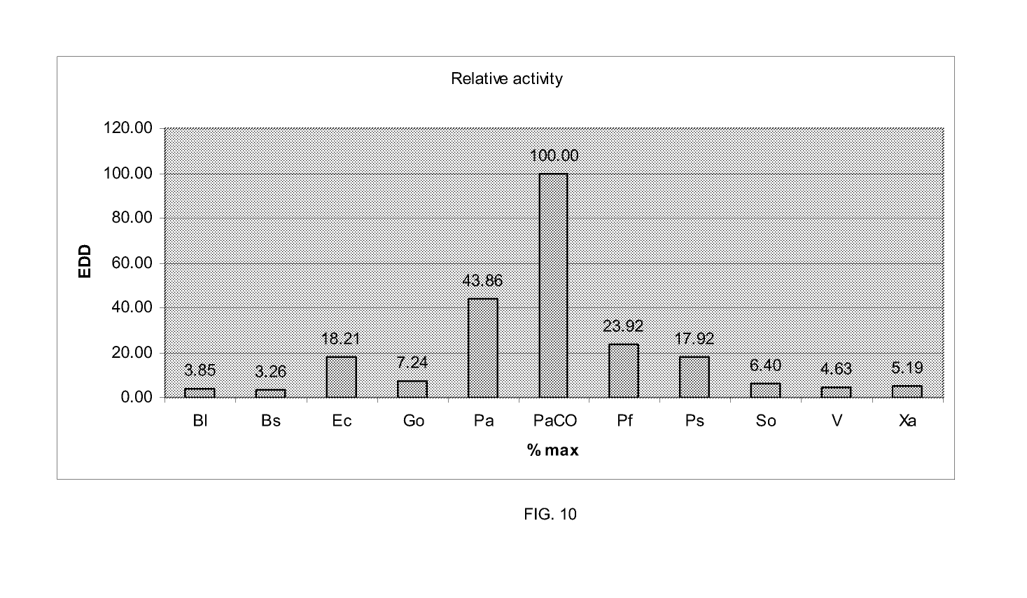

E. coli expressed EDD was prepared and confirmed by western blot analysis as shown in FIG. 8. The expected size of EDD is approximately 66 kilodaltons (kDa). A band of approximately that size (e.g., as determined by the nearest sized protein standard of approximately 60 kDa) was identified by western blot. The E. coli expressed EDD was used with S. cerevisiae expressed EDA's to evaluate the EDA activities. The results of EDA kinetic assays are presented in the table below.

| EDD/EDA | slope | % max |
|---|---|---|
| EC/EC | 0.3467 | 100.00 |
| EC/SO | 0.1907 | 55.00 |
| EC/BS | 0.0897 | 25.87 |
| EC/GO | 0.0848 | 24.46 |
| EC/PCO | 0.084 | 24.23 |
| EC/PA | 0.0533 | 15.37 |
| EC/PE5 | 0.0223 | 6.43 |
| EC/PE10 | 0.0218 | 6.29 |
| EC/SD | 0.015 | 4.33 |
| EC/PS | 0.0135 | 3.89 |
| EC/BL | 0.0112 | 3.23 |
| EC/ZM | 0.0109 | 3.14 |
| EC/PF | 0.0082 | 2.37 |
| EC/V | 0.0074 | 2.13 |
| EC/XA | 0.0065 | 1.87 |
| EC/PE15 | 0.005 | 1.44 |

In the results presented above, the slope of the E. coli (EC) EDA is outside the linear range for accurate detection, and is therefore underestimated. For the other EDA's, when compared to the E. coli EDA, the calculated percentage of maximum activity (e.g., % max) is overestimated, however the slopes are accurate. The results of this experiment indicate that the E. coli EDA has higher activity as compared to the other EDA activities evaluated herein, and is approximately 16-fold more active than the EDA from P. aeruginosa. EDA's from X. anoxopodis and a chimera between E. coli EDA and P. aeruginosa (e.g., PE15) show less activity than the vector control. Codon-optimized EDA from P. aeruginosa showed a slight improvement over the native sequence, however chimeric versions (e.g., PE5, PE10, PE15) showed less activity than native. The experiments were repeated using 100 μg of EDD and 25 μg of EDA cell lysates in each reaction (unless otherwise noted, such as 5 μg of E. coli EDA). The reactions in the repeated experiment all were in the linear range of detection and the results of these additional kinetic assays are shown graphically in FIG. 9, and in the table below. E. coli EDA was again found to be the most active of those EDA's tested.

| EDA | slope | % max |
|---|---|---|
| EC | 0.462 | 100.00 |
| SO | 0.128 | 27.71 |
| GO | 0.0544 | 11.77 |
| PCO | 0.0539 | 11.67 |
| BS | 0.0505 | 10.93 |
| PA | 0.0273 | 5.91 |
| V | 0.0006 | 0.13 |

### Example 11

### EDA Activity Assays using Various EDD Genes Over-Expressed in E. coli

Assays to evaluate EDA activity were performed in vitro using various over-expressed EDD from E. coli and the various isolated EDA's expressed in S. cerevisiae. EDA and EDD extracts were prepared as described in Example 10. Each activity assay reaction contained 50 mM Tris-HCl, pH 7, 10 mM MgCl₂, 0.15 mM NADH, 15 μg LDH, saturating amounts of EDD determined empirically (usually ˜100 μg), 1-100 μg EDA (depending on level of activity), and 1 mM 6-phosphogluconate. Reactions were started by the addition of 6-phosphogluconate and monitored for 5 minutes at 30° C. The results are illustrated graphically in FIG. 10. FIG. 10 shows the relative activity of various EDD sources. The codon-optimized PAO1 EDD was found to have the highest amount of activity.

### Example 12

### Examples of Embodiments

Listed hereafter are non-limiting examples of certain embodiments.

A1. A method for generating a combinatorial library of nucleic acids, which comprises:
(a) providing a group of polynucleotides comprising two or more polynucleotide subgroups, wherein:
   (i) each polynucleotide in each polynucleotide subgroup encodes a polypeptide of a corresponding polypeptide subgroup;
   (ii) each polypeptide in a particular polypeptide subgroup share an activity; and
   (iii) polypeptides of one polypeptide subgroup have a different activity from the polypeptides of every other polypeptide subgroup; and
(b) assembling the polynucleotides into a nucleic acid library.
   A2. The method of embodiment A1, wherein each nucleic acid of the nucleic acid library includes one polynucleotide species from each of the two or more polynucleotide subgroups.
   A3. The method of embodiment A1 or A2, wherein each nucleic acid of the nucleic acid library comprises polynucleotide species linked in series.
   A4. The method of embodiment A3, wherein the polynucleotide species are separated from one another by linkers.
   A5. The method of any one of embodiments A1-A4, wherein the polynucleotide species are in operable linkage with one or more promoters.
   A6. The method of embodiment A5, wherein the polynucleotide species are in operable linkage with one promoter.
   A7. The method of embodiment A5, wherein each polynucleotide species is in operable linkage with a separate promoter.
   A8. The method of any one of embodiments A1-A7, wherein there are 50 or fewer polynucleotide subgroups.
   A9. The method of any one of embodiments A2-A8, wherein the polynucleotides are assembled using an oligonucleotide assembly process.
   A10. The method of any one of embodiments A1-A9, wherein the nucleic acid library includes 60% or more of all possible subgroup species combinations.
   A11. The method of any one of embodiments A1-A10, wherein the polynucleotides comprise complementary DNA (cDNA).
   A12. The method of embodiment A11, wherein the polynucleotides consist essentially of complementary DNA (cDNA).
   A13. The method of any one of embodiments A1-A12, which comprises inserting nucleic acid of the library into an expression construct.
   A14. The method of embodiment A13, which comprises inserting the expression construct into an organism.
   A15. The method of embodiment A14, which comprises determining the amount of a target product produced by the organism.
   A16. The method of any one of embodiments A1-A12, which comprises inserting nucleic acid of the library into a yeast artificial chromosome.
   A17. The method of embodiment A16, which comprises inserting the artificial chromosome in a yeast.
   A18. The method of embodiment A17, which comprises determining the amount of a target product produced by the yeast.
   A19. The method of any one of embodiments A1-A12, which comprises inserting nucleic acid of the library into genomic DNA of an organism.
   A20. The method of embodiment A19, which comprises determining the amount of a target product produced by the organism.
   B1. A nucleic acid library comprising a group of polynucleotides that includes two or more polynucleotide subgroups, wherein:
(i) each polynucleotide in each polynucleotide subgroup encodes a polypeptide of a corresponding polypeptide subgroup;
(ii) each polypeptide in a particular polypeptide subgroup share an activity; and
(iii) polypeptides of one polypeptide subgroup have a different activity from the polypeptides of every other polypeptide subgroup.
   B2. The nucleic acid library of embodiment B1, wherein each nucleic acid of the nucleic acid library includes one polynucleotide species from each of the two or more polynucleotide subgroups.
   B3. The nucleic acid library of embodiment B1 or B2, wherein each nucleic acid of the nucleic acid library comprises polynucleotide species linked in series.
   B4. The nucleic acid library of embodiment B3, wherein the polynucleotide species are separated from one another by linkers.
   B5. The nucleic acid library of any one of embodiments B1-B4, wherein the polynucleotide species are in operable linkage with one or more promoters.
   B6. The nucleic acid library of embodiment B5, wherein the polynucleotide species are in operable linkage with one promoter.
   B7. The nucleic acid library of embodiment B5, wherein each polynucleotide species is in operable linkage with a separate promoter.
   B8. The nucleic acid library of any one of embodiments B1-B7, wherein there are 50 or fewer polynucleotide subgroups.
   B9. The nucleic acid library of any one of embodiments B2-B8, wherein the polynucleotides are assembled using an oligonucleotide assembly process.
   B10. The nucleic acid library of any one of embodiments B1-B9, wherein the nucleic acid library includes 60% or more of all possible subgroup species combinations.
   B11. The nucleic acid library of any one of embodiments B1-B10, wherein the polynucleotides comprise complementary DNA (cDNA).
   B12. The nucleic acid library of embodiment B11, wherein the polynucleotides consist essentially of complementary DNA (cDNA).
   C1. An isolated expression construct comprising a nucleic acid from a nucleic acid library produced by a method of any one of embodiments A1-A12.
   C2. An isolated expression construct comprising a nucleic acid from a nucleic acid library of any one of embodiments B1-B12.
   D1. An organism that comprises a nucleic acid from a nucleic acid library produced by a method of any one of embodiments A1-A12.
   D2. An organism prepared by a method of any one of embodiments A14, A17 or A19.
   D3. An organism that comprises a nucleic acid of a nucleic acid library of any one of embodiments B1-B12.
   D4. An organism that comprises an expression construct of embodiment C1 or C2.
   D5. The organism of any one of embodiments D1-D4, which is a prokaryote.
   D6. The organism of embodiment D5, which is a bacterium.
   D7. The organism of any one of embodiments D1-D4, which is a eukaryote.
   D8. The organism of embodiment D7, which is a fungus.
   D8. The organism of embodiment D7, which is a yeast.
   D9. The organism of embodiment D7, which is a mammalian cell.
   D10. The organism of embodiment D7, which is an insect cell.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the claimed technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of the claimed technology.

Certain embodiments of the technology are set forth in the claim(s) that follow(s).

1-20. (canceled)

21. A nucleic acid library comprising a group of polynucleotides that includes two or more polynucleotide subgroups, wherein:
(i) each polynucleotide in each polynucleotide subgroup encodes a polypeptide of a corresponding polypeptide subgroup;
(ii) each polypeptide in a particular polypeptide subgroup share an activity; and
(iii) polypeptides of one polypeptide subgroup have a different activity from the polypeptides of every other polypeptide subgroup.

22. The nucleic acid library of claim 21, wherein each nucleic acid of the nucleic acid library includes one polynucleotide species from each of the two or more polynucleotide subgroups.

23. The nucleic acid library of claim 21, wherein each nucleic acid of the nucleic acid library comprises polynucleotide species linked in series.

24. The nucleic acid library of claim 23, wherein the polynucleotide species are separated from one another by linkers.

25. The nucleic acid library of claim 21, wherein the polynucleotide species are in operable linkage with one or more promoters.

26. The nucleic acid library of claim 25, wherein the polynucleotide species are in operable linkage with one promoter.

27. The nucleic acid library of claim 25, wherein each polynucleotide species is in operable linkage with a separate promoter.

28. The nucleic acid library of claim 21, wherein there are 50 or fewer polynucleotide subgroups.

29. The nucleic acid library of claim 21, wherein the polynucleotides are assembled using an oligonucleotide assembly process.

30. The nucleic acid library of claim 21, wherein the nucleic acid library includes 60% or more of all possible subgroup species combinations.

31. The nucleic acid library of claim 21, wherein the polynucleotides comprise complementary DNA (cDNA).

32. The nucleic acid library of claim 31, wherein the polynucleotides consist essentially of complementary DNA (cDNA).

33. (canceled)

34. An isolated expression construct comprising a nucleic acid from a nucleic acid library of claim 21.

35. (canceled)

36. (canceled)

37. An organism that comprises a nucleic acid of a nucleic acid library of claim 21.

38. An organism that comprises an expression construct of claim 34.

39. The organism of claim 37, which is a prokaryote.

40. The organism of claim 39, which is a bacterium.

41. The organism of claim 37, which is a eukaryote.

42. The organism of claim 41, which is a fungus.

43. The organism of claim 41, which is a yeast.

44. The organism of claim 41, which is a mammalian cell.

45. The organism of claim 41, which is an insect cell.
